(19) United States
(12) Patent Application Publication
(10) Pub. No.: US 2025/0296961 A1
(43) Pub. Date: Sep. 25, 2025

(54) ANTIMICROBIAL PEPTIDES FOR REMINERALIZATION OF TEETH AND PREVENTION OF DENTAL CARIES

(71) Applicant: The University of Hong Kong, Hong Kong (CN)

(72) Inventors: Hai Ming Wong, Hong Kong (CN); Quan Li Li, Hong Kong (CN); Li Zhou, Hong Kong (CN); Qing Liu, Hong Kong (CN)

(21) Appl. No.: 19/051,751

(22) Filed: Feb. 12, 2025

Related U.S. Application Data
(63) Continuation-in-part of application No. 18/546,733, filed on Aug. 16, 2023.
(60) Provisional application No. 63/149,961, filed on Feb. 16, 2021.
(60) Provisional application No. 63/559,440, filed on Feb. 29, 2024.
(60) Provisional application No. 63/669,288, filed on Jul. 10, 2024.

(51) Int. Cl. C07K 14/00; A61K 8/00; A61Q 11/00

(52) U.S. Cl. CPC C07K 14/001; A61Q 11/00; A61K 8/00

(57) ABSTRACT

Peptides that bind with high affinity to oral cavity surfaces, such as tooth enamel, have been discovered. The peptides exhibit antibacterial and mineralizing abilities, and are useful in many aspects of oral care. The peptides include the amino acid sequence AKRHHGYKRKFH-SpSp. The peptides can be included in oral care compositions such as toothpastes and mouthwashes. The oral care compositions can be used in methods to treat or prevent diseases or conditions induced by acidophilic oral bacteria. Exemplary methods include reducing or preventing tooth decay or demineralization, biofilm or dental plaque formation, and/or bacterial adhesion to a tooth surface; and promoting mineralization of teeth surfaces.

### CROSS REFERENCE TO RELATED APPLICATIONS

This application is a continuation-in-part of U.S. application Ser. No. 18/546,733 filed Aug. 16, 2023, as a National Phase application under 35 U.S.C. 371 of PCT/CN2022/072965, filed Jan. 20, 2022, which claims priority to and the benefit of U.S. Provisional Patent Application No. 63/149,961 filed on Feb. 16, 2021, and it claims priority to U.S. Provisional application No. 63/669,288 filed on 29 Feb. 2024 and U.S. Provisional application No. 63/559,440 filed on 10 Jul. 2024, the entire contents of which are incorporated herein by reference for all purpose in their entirety.

### REFERENCE TO SEQUENCE LISTING

The Sequence Listing XML submitted as a file named “UHK_01004-01446CIP_ST26”, created on Jun. 17, 2025, and having a file size of 19,085 bytes is hereby incorporated by reference pursuant to 37 C.F.R. § 1.834(c)(1).

### FIELD OF THE INVENTION

The disclosed invention is generally in the field of oral care, and specifically, relates to methods and compositions for preventing and treating dental caries.

### BACKGROUND OF THE INVENTION

Tooth decay is a significant oral health problem that affects millions of individuals around the world. Tooth decay can cause significant tooth damage compromising the esthetics and function of teeth, requiring extensive dental treatment. Due to a fear of the dental drill and anesthetic injections, most people do not attend dental appointments regularly. As such, a majority of caries lesions are not restored at the initial stage and gradually become larger and symptomatic. Traditional caries treatment is an invasive therapy, including preparing a cavity on sound dental tissue and restoring it with conventional materials, such as amalgam and composite resin. However, due to the differences in properties between dental tissue and artificial materials, the long-term performance of the restored tooth is suboptimal. Thus, prevention and reversal of dental caries prior to cavity formation presents a difficult challenge for dentistry.

Dental caries results from an ecological imbalance within the caries pathogen biofilm that favors specific acidogenic pathogens, such as Streptococcus mutans (S. mutans), which decompose sugars to produce acids which interrupt the tooth surface mineralization balance and induce demineralization of dental hard tissues. For example, when bacteria metabolize fermentable carbohydrates, acids are produced as a byproduct that lower the pH value of oral and plaque fluids, therefore creating an acidic oral environment. The bacterial acids (including particularly lactic acid) may then, among other things, leach minerals out of tooth structures within the oral cavity. Dental caries is typically characterized by this demineralization of mineral components and the decomposition of the organic matrix from one or more calcified structures of a tooth: enamel, dentin, or cementum. Therefore, caries is theoretically preventable and repairable, for example by inhibiting caries pathogen biofilm formation, reducing dental hard tissue demineralization, and inducing its remineralization.

There are numerous strategies for caries management. The use of chemical surface modifications as nonspecific coatings, such as polyethylene glycol and zwitterion, has demonstrated anti-adhesive properties in vitro. However, their low surface densities and sensitivity to oxidative damage might result in failure of antibacterial adhesion for long-term applications. Commercial products, e.g., toothpaste and mouthwashes, containing chemicals such as antibiotics, chlorhexidine, and fluorides are commonly used to prevent caries pathogen biofilm formation. However, antibiotics are unsuitable for daily use because of the potential to result in antibiotic resistance and subsequent side effects, while the efficacy of chlorhexidine is low. Fluorides show certain antibacterial activities only at very high concentrations. It is difficult to continuously sustain their topical concentrations on the tooth, and their toxicity is also a concern. Some fluoride- and calcium phosphate-based reagents such as casein phosphopeptide-amorphous calcium phosphate have been used to induce mineral deposits on tooth surfaces, but the deposits do not actually become part of the enamel microstructure. Furthermore, most of the existing anti-caries agents are mono-functional. Thus, there is an urgent and ongoing need for agents and methods to improve prevention and treatment of dental caries.

Therefore, it is an object of the invention to provide compositions and methods for the prevention and treatment of dental caries (tooth decay).

It is an object of the invention to provide antibacterial and/or mineralizing compositions useful for dental care.

It is an object of the invention to provide compositions and methods to prevent adhesion of microorganisms to a tooth surface.

It is another object of the invention to provide compositions and methods for protecting teeth against an acidic environment.

It is a further object of the invention to provide compositions and methods to prevent or reduce tooth demineralization and/or induce tooth remineralization.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### BRIEF SUMMARY OF THE INVENTION

Peptides that bind with high affinity to oral cavity surfaces, such as tooth enamel, have been discovered. The peptides exhibit both anti-biofouling and mineralizing abilities, and as such, are useful in many aspects of oral care.

In particular, disclosed is an isolated peptide containing at least two parts: an antimicrobial domain and remineralization enhancing domain such as, a phosphoserine domain. The antimicrobial domain can include a histatin 5 segment. A histatin 5 segment can be or include a histatin 5 protein, a variant thereof, a portion thereof, or a variant of a portion thereof. Typically, the antimicrobial domain is histatin 5 (e.g., human histatin 5) or a portion thereof. For example, in some forms, the histatin 5 or portion thereof includes the amino acid sequence DSHAKRHHGYKRKFHEKHHSHRGY (SEQ ID NO:1) or a sequence having at least 90% sequence identity to DSHAKRHHGYKRKFHEKHHSHRGY (SEQ ID NO:1).

Preferably, the antimicrobial domain is or contains a portion of human histatin 5, such as AKRHHGYKRKFH (SEQ ID NO:2) or a sequence having at least 90% sequence identity to AKRHHGYKRKFH (SEQ ID NO:2) (P113).

The phosphoserine domain is or includes a phosphorylated serine. In some forms, the phosphoserine domain includes two or more phosphorylated serines (Sp). Typically, the phosphoserine domain is positioned at the C-terminus of the isolated peptide. A preferred peptide of the disclosure includes the amino acid sequence AKRHHGYKRKFH-SpSp (SEQ ID NO:4), wherein each Sp independently represents a phosphorylated serine, and thus, SEQ ID NO: 4 includes two phosphoserines/diphophoserine (DPS).

In some forms, the peptide is a multifunctional antimicrobial (AMP) with three parts: a non-specific AMP, P-113, a specific targeting domain, C8, and a remineralization enhancing domain. In some forms, the mineralization domain includes two phosphoserines (DPS), and 24 amino acids, DSSDSSDSSDSSDSSDSSDSSDSS (8DSS) (SEQ ID NO: 11)^{18,19}. The multi-functional AMPs disclosed herein include in some forms, C8-P-113 (SEQ ID NO:12), DPS-C8-P-113 (SEQ ID NO:8) or 8DSS-C8-P-113 (SEQ ID NO: 10), or a sequence having at least 90% sequence identity to C8-P-113, DPS-C8-P-113 or 8DSS-C8-P-113.

The agent C8-P-113, can include two components: a non-specific AMP (P-113), and a competence stimulating peptide (C8).

The agent DPS-C8-P-113, can include three components: a non-specific AMP (P-113), a competence stimulating peptide (C8), and an enhancing remineralization domain (DPS).

The agent 8DSS-C8-P-113, can include three components: a non-specific AMP (P-113), a competence stimulating peptide (C8), and an enhancing remineralization domain (8DSS).

Preferably, the peptide is capable of binding to a tooth surface, such as enamel. In some forms, upon contact with an oral cavity (e.g., tooth surface) the isolated peptide binds to tooth enamel, reduces or prevents tooth decay or demineralization, reduces or prevents bacterial biofilm formation on a tooth surface, reduces or prevents dental plaque formation, reduces or prevents bacterial adhesion to a tooth surface, promotes (e.g., induces or increases) mineralization of a tooth surface, inhibits growth and/or proliferation of bacteria in the oral cavity, or combinations thereof. For example, in some forms, upon coating or otherwise being applied to a tooth surface the peptide attracts calcium and/or phosphate ions (e.g., from saliva) to promote generation of new crystal deposits on the tooth surface.

Also disclosed are composition with enamel-like structure including fluorapatite (FAP) and low-molecular-weight polyacrylic acid (LPAA) that can be used in methods to treat dental restoration. The anionic polymer, LPAA, can regulate the balance between nucleation and the crystal growth of FAP and can improve the mechanical properties of enamel-like structure via the bonding effect among the adjacent crystals by carboxy group. In certain embodiments, the crystals form a prism-like structure. The composition including FAP-LPAA can prevent and/or reduce the proliferation of bacteria on the enamel-like structure surface. In certain embodiments, Streptococcus mutans on the FAP-LPAA surface could be killed within 12 hours. LPAA is safe for human beings. Thus, LPAA can be used as a useful additive when antibacterial enamel-like FAP was generated for dental restoration.

Also disclosed are compositions including one or more of the disclosed peptides. For example, pharmaceutical compositions including an effective amount of the peptide or a plurality of copies of the peptide and a pharmaceutically acceptable excipient are provided. Typically, the excipient is acceptable for administration to oral cavity. Oral care compositions or devices including the isolated peptide or a plurality of copies of the isolated peptide are provided. Exemplary compositions or devices include, without limitation, mouthwashes, toothpastes, dental creams, oral sprays, rinses, gargles, gums, tablets, oral lozenges, dental floss, tooth whitening strips or solutions, cleaning solutions, toothpicks, toothbrushes, varnishes, and restorative materials such as composite resin.

Methods of using the peptides and compositions thereof are also provided. An exemplary method involves applying a disclosed oral care composition or device containing the peptide to the teeth and/or oral cavity of a subject. In some forms, applying the peptide to the teeth and/or oral cavity of a subject can be used to reduce or prevent tooth decay, repair tooth decay, reduce or prevent enamel demineralization, promote enamel mineralization, or combinations thereof.

For example, disclosed is a method of reducing or preventing and/or repairing tooth decay in a subject in need thereof by contacting the subject's teeth and/or oral cavity with a peptide having the sequence AKRHHGYKRKFH-SpSp (SEQ ID NO:4) or a sequence having at least 90% sequence identity to AKRHHGYKRKFH-SpSp (SEQ ID NO:4). Also provided is method of reducing or preventing enamel demineralization and/or promoting enamel mineralization in a subject in need thereof by contacting the subject's teeth and/or oral cavity with a peptide having the sequence AKRHHGYKRKFH-SpSp (SEQ ID NO:4) or a sequence having at least 90% sequence identity to AKRHHGYKRKFH-SpSp (SEQ ID NO:4).

In some forms the amount of the disclosed peptides is effective to reduce the levels of planktonic S. mutans without disrupting the oral normal flora.

In some forms, the amount of the disclosed peptides is effective to prevent S. mutans' adhesion.

In some forms, the amount of the disclosed peptides is effective to form a hydrogel state, eradicating S. mutans' biofilm, adhering on the tooth surface (thus protecting it against acid injury), and facilitating the repair of demineralized hard tissue.

In some forms of the foregoing methods, the peptide or a plurality of copies of the peptide are included in a composition or device selected from mouthwash, toothpaste, dental cream, oral spray, a rinse, a gargle, a gum, a tablet, an oral lozenge, dental floss, a tooth whitening strip or solution, a cleaning solution, a toothpick, a toothbrush, a varnish, or composite resin. In some forms, the peptide is in an amount effective to reduce or prevent tooth decay, reduce or prevent bacterial biofilm formation, reduce or prevent dental plaque formation, reduce or prevent bacterial adhesion to tooth surfaces, inhibit growth and/or proliferation of oral bacteria, induce or increase enamel mineralization, or combinations thereof. In some forms, the bacteria contained in the biofilm, the bacteria whose adhesion is reduced or prevented, and/or whose growth and/or proliferation is inhibited is selected from Streptococcus mutans, Streptococcus faecalis, Streptococcus sobrinus, Lactobacillus acidophilus, Lactobacillus casei, or combinations thereof.

Additional advantages of the disclosed methods will be set forth in part in the description which follows, and in part will be understood from the description, or can be learned by practice of the disclosed methods and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed method and compositions.

FIG. 1 is a graph showing adsorption time of P-113, P-113-DPS, P-113-DA, P-113-SK, and Sp-H5 on tooth enamel surfaces. Where indicated, a significant concentration difference was observed in the time point with an icon (*, #, &, $ and @, respectively, P<0.01) compared to the 0 min time point for the respective peptide.

FIG. 2 is a graph showing viable counts (left ordinate and point plot) and percentage reduction of viability counts (right ordinate and bar chart) of S. mutans biofilms after 24 hours of exposure to BHI, P-113, P-113-DPS, P-113-DA, P-113-SK and Sp-H5 solutions at the indicated concentrations. The viable counts with an icon (*, #, $ or @) indicate a P<0.001, compared with BHI.

FIG. 3 is a graph showing viable counts of S. mutans on enamel surfaces coated with P-113, P-113-DPS, P-113-DA, P-113-SK, and Sp-H5 after 5 hours of incubation. *P<0.001, compared with BHI.

FIG. 4 is a graph showing the amount of Ca/P loss on enamel surfaces in the demineralization solutions after coating with the indicated peptides. The dotted bar indicates the Ca loss, and the crosshatched bar denotes the P loss. Different capital letters show significant differences (A<B, C<D<E<F<G, P<0.001).

FIG. 5A is a schematic representation of the assay to determine mineral gain after 24 h in remineralization solutions. FIG. 5B is a graph showing the amount of Ca/P gain on enamel surfaces in the remineralization solutions after coating with the indicated peptides. The brick bar indicates the Ca gain, and the diamond bar denotes the P gain. Different capital letters show significant differences (A>B>C, D>F, D>G, E>G, P<0.001).

FIG. 6A-6B shows cytocompatibility evaluation of peptides using MC3T3-E1. FIG. 6A is a graph showing proliferation of MC3T3-E1 treated with peptides at different concentrations for 24 h. *p<0.001, compared with control. FIG. 6B is a graph showing proliferation of MC3T3-E1 on 8 μM mL-1 peptide-coated enamel surfaces for 1 and 3 days.

FIGS. 7A-7F are graphs showing the centroid distance between P-113 and HA (FIG. 7A), the binding energy between P-113 and HA (FIG. 7B), the binding energy between the Ca2+(red), OH— (gray), and PO43- (blue) of HA and P-113 (FIG. 7C), P-113-DPS (FIG. 7D), P-113-DA (FIG. 7E), and P-113-SK (FIG. 7F), respectively.

FIGS. 8A-8H are graphs showing the binding energy between P-113 and the ions (FIG. 8A), HA and the ions in the P-113-HA complex (FIG. 8B), P-113-DPS and the ions (FIG. 8C), HA and the ions in the P-113-DPS-HA complex (FIG. 8D), P-113-DA and the ions (FIG. 8E), HA and the ions in the P-113-DA-HA complex (FIG. 8F), P-113-SK and the ions (FIG. 8G), and HA and the ions in the P-113-SK-HA complex (FIG. 8H).

FIGS. 9A-9H are graphs showing the binding energy between P-113 and the ions (FIG. 9A), HA of the P-113-HA complex and the ions (FIG. 9B), P-113-DPS and the ions (FIG. 9C), HA of the P-113-DPS-HA complex and the ions (FIG. 9D), P-113-DA and the ions (FIG. 9E), HA of the P-113-DA-HA complex and the ions (FIG. 9F), P-113-SK and the ions (FIG. 9G), and HA of the P-113-SK-HA complex and the ions (FIG. 9H).

FIG. 10 is a schematic depicting mechanisms of action of P-113-DPS. (1) P-113-DPS adheres to the enamel surface via positively charged amino acids (blue circles: N-ter, Lys2, Arg3, Arg9, and Lys10) attracted to negatively charged PO43- of HA. (2) P-113-DPS kills planktonic S. mutans. (3) Coating of P-113-DPS on enamel inhibits the adhesion of S. mutans. (4) Coating of P-113-DPS on enamel protects the enamel surface against demineralization. (5) Coating of P-113-DPS on enamel increases the thickness of the regenerated crystal layer. Ser13-p and Ser14-p are in the red circles. Carboxyl is in the green circle. The green dotted lines represent coordinate bonds, and the pink dotted lines represent hydrogen bonds.

FIG. 11A. Viable counts of S. mutans adhesion on peptide-coated enamel surfaces after 5 h of incubation. FIG. 11B. Viable counts (left ordinate and point plot) and percentage reduction in viable counts (right ordinate and bar chart) of S. mutans biofilms after 24 h of exposure to peptide solutions with different concentrations. *p<0.05, **p<0.01, ***p<0.001, compared with control group. 8DSS-C8-P-113 hydrogel peptide was stained green and indicated by arrows.

FIGS. 12A-12K Characterization, TEM micrographs, stability, and cytocompatibility of peptides. FIGS. 12A-12C show TEM micrographs of C8-P-113, DPS-C8-P-113 and 8DSS-C8-P-113, respectively. (FIG. 12D-12E) FE-SEM micrographs of 8DSS-C8-P-113 hydrogel (×50,000) and 8DSS-C8-P-113 hydrogel (×10,000). FIG. 12F show changes in remaining percentage of peptides incubated in human saliva at 37° C. during 6 h. FIG. 12G shows changes in remaining percentage of 8DSS-C8-P-113 hydrogel incubated in flow water at the rate of 30 mL h-1 during 2 h. FIG. 12H shows cell survival rate after cells exposed at peptide solution for 24 h. *p<0.05, compared with control. FIG. 12I shows proliferation of MC3T3-E1 on peptide-coated enamel surfaces (at MBC concentration) for 1 and 3 days.

FIGS. 13A-13U. Remineralization assay. (FIGS. 13A-13F) FE-SEM micrographs (×10,000) of the remineralization cross-sections in C8-P-113 (MBC), DPS-C8-P-113 (MBC), 8DSS-C8-P-113 (MBC), 8DSS-C8-P-113 (16 μm mL⁻¹), agarose, and control group on the 1st day, respectively. (FIGS. 13G-13L) FE-SEM micrographs (×2000) in different groups on the 3rd day. (FIGS. 13M-13R) FE-SEM micrographs (×1000) in different groups on the 7th day. (FIG. 13S) Magnification of micrograph (FIG. 13P) (×20,000). The big brace indicates the thickness of the 8DSS-C8-P-113 hydrogel layer. (FIG. 13T) Magnification of micrograph (FIG. 13U) (×10,000). (FIG. 13U) Magnification of micrograph (FIG. 13R) (×2000). The arrows indicate the thickness of needle-like crystals. (FIG. 13V) Vicker's hardness of different surface treated-enamel specimens after 1 day, 3 days, and 7 days of incubation in the remineralization solution. *p<0.05, **p<0.01, ***p<0.001, compared with control group in the same day; for each group of bars (D1, D2 and Day3), the groups as shown as follows from left to right-C8-P-113 (MBC); DPS-C8-P-113 (MBC); 8DSS-C8-P-113 (MBC); 8DSS-C8-P-113 (16 μm mL⁻¹); Agarose and control.

FIGS. 14A-14H show cross-section of lesions on the enamel surface in the artificial caries models. (FIGS. 14A-14F) MicroCT graphs (×10,000) of the demineralization cross-sections in C8-P-113 (MBC), DPS-C8-P-113 (MBC), 8DSS-C8-P-113 (MBC), 8DSS-C8-P-113 (16 μM mL⁻¹), agarose, and control group, respectively. (FIG. 14G) Lesion depth on enamel surfaces. (FIG. 14H) Bone mineral density of lesion on enamel surfaces. *p<0.05, **p<0.01, ***p<0.001, compared with control group.

FIGS. 15A-15H. The mechanism of antibacterial activity of 8DSS-C8-P-113 evaluated by LDH assay, water-insoluble EPS, and transcriptomic analysis of S. mutans. (FIG. 15A) LDH activity in the different concentrations of 8DSS-C8-P113 relative to that of the control group. (FIG. 15B) The quantification of water-insoluble EPS of S. mutans biofilms in the different concentrations of 8DSS-C8-P113. (FIG. 15C) Quantitative data of bacterial and EPS biomass in the different concentrations of 8DSS-C8-P113. *p<0.05, **p<0.01, ***p<0.001, compared with control group. (FIG. 15D) The volcano plot of DEGs (|log₂fold change|>1 and P<0.05) in S. mutans between the control group and 8DSS-C8-P-113-treated group. The abscissa indicates the fold-change in gene expression, while the ordinate indicates significant statistical differences. Black dots in upper right panel (“Up” in the figure) represent significantly upregulated genes, black dots in upper left panel (“Down” in the figure) represent significantly downregulated genes, and gray dots represent insignificantly differentially expressed genes. (FIG. 15E) Heatmap of the DEGs between control and 8DSS-C8-P-113-treated group. Darker gray represents a higher expression level while lighter gray represents a lower expression level. (FIG. 15F) GO enrichment analysis of the upregulated and downregulated genes between control and 8DSS-C8-P-113-treated group. The x-axis shows the significant statistical differences, and the y-axis indicates the GO terms enriched in the upregulated and downregulated genes. Dark dotted and light dotted patterns indicate the up- and down-regulated genes, respectively. (FIG. 15G) KEGG pathway enrichment analysis of the upregulated and downregulated genes between control and 8DSS-C8-P-113-treated group. The x-axis shows the significant statistical differences, and the y-axis indicates the KEGG pathways enriched in the upregulated and downregulated genes. Dark dotted and light dotted patterns indicate the up- and down-regulated genes, respectively.

FIG. 16A. Average distance between 8DSS-C8-P-113 clusters and the surface of HAP. (FIG. 16B) The atomic contact numbers between 8DSS-C8-P-113 hydrogel and HAP surfaces, Ca²⁺ ions and HAP surfaces, H₂PO₄⁻ and HAP surfaces, and HPO₄²⁻ and HAP surfaces.

FIG. 17 Schematic diagram illustrating the functions of 8DSS-C8-P-113 in this study. (1) 8DSS-C8-P-113 molecules form a hydrogel state and adhere to the tooth surface. The molecule structure consists of a blue part representing 8DSS, a red part representing C8, and a green part representing P-113. (2) Potential antibacterial mechanism of 8DSS-C8-P-113 on S. mutans is as follows: 1) inducing cell membrane perforation, 2) C8 activating a transmembrane receptor and interfering with QS, 3) interfering with peptidoglycan biosynthesis, 4) interfering with glycolysis, 5) inhibiting DNA polymerase activity, and 6) inhibiting protein modification. (3) 8DSS-C8-P-113 hydrogel induces Ca²⁺, H₂PO₄⁻, and HPO₄²⁻ adhering to HAP (001), forming a remineralization layer. (4) 8DSS-C8-P-113 hydrogel adsorbs on HAP (100), inhibits S. mutans' adhesion, and reduces tooth demineralization.

FIG. 18A-18E. Evaluation of caries prevention in a rat dental caries model. (FIG. 18A) schematic diagram of the in vivo experimental procedure. (FIG. 18B) Quantification of S. mutans CFU in each group. (FIG. 18C) Keyes' score of the sulcal surface. (FIG. 18D) Keyes' score of the smooth surface. (FIG. 18E) Body weights of rats. *p<0.05, **p<0.01, compared with HEPES group.

FIG. 19. Growth curve of S. mutans in the ½×MBC C8-P-113, ½×MBC DPS-C8-P-113, ½×MBC 8DSS-C8-P-113, or BHI solutions.

### DETAILED DESCRIPTION OF THE INVENTION

The disclosed methods and compositions can be understood more readily by reference to the following detailed description of particular embodiments and the Examples included therein and to the Figures and their previous and following description.

Microorganisms reside in the oral cavity and easily accumulate on tooth surfaces. Some bacterial microorganisms produce a local acidic environment, which ultimately causes demineralization of tooth hard tissues, such as enamel, thereby causing dental caries. Despite the prevalence of oral care products, there is a continuing need for agents that can prevent microorganism adhesion to tooth surfaces, protect teeth against bacteria-induced acidic environment, and reverse demineralization of teeth.

To this end, peptides that bind with high affinity to oral cavity surfaces, such as tooth enamel, have been developed. The working Examples describe development and functional characterization of a number of peptides, including their ability to adsorb to enamel, to prevent bacterial biofilm formation, to kill planktonic bacteria, to reduce demineralization, and to promote remineralization. In particular, the peptide P-113-DPS which contains P-113, a fragment of the antimicrobial histatin 5 peptides, and a diphosphserine domain (which can chelate free Ca²⁺ to promote tooth in situ mineralization) grafted to the C-terminus of P-113 outperformed all other peptides tested. The antibacterial activity of P-113-DPS was stronger than other peptides such as P-113-DA, P-113-SK, and P-113. Among the peptides tested, P-113-DPS showed the highest binding affinity to the tooth surface, the strongest suppression of acid-induced corrosion, and the thickest newly regenerated crystals. P-113-DPS adsorbs onto enamel surfaces within 20 minutes, and subsequent adhesion of microorganisms to the enamel surface is reduced. Thus, P-113-DPS and functionally equivalent peptides can be used to promote overall oral health, including preventing or treating dental caries.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. Definitions

As used herein in reference to the peptides, the term “isolated” means a peptide that is in a form that is relatively free from material such as contaminating polypeptides, lipids, nucleic acids and other cellular material that normally is associated with the peptide in a cell or that is associated with the peptide in a library or in a crude preparation. A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography.

As used herein, the term “mineralization” refers to the introduction of or restoration of minerals to dental hard tissues within the oral cavity. The term not only includes remineralizing areas that are hypomineralized, but may also include mineralizing and strengthening tissues that may be healthy, existing, and/or newly erupted.

The term “tooth surface” refers to a surface comprised of tooth enamel (typically exposed after professional cleaning or polishing) or tooth pellicle (an acquired surface comprising salivary glycoproteins). As used herein, the terms “enamel” and “tooth enamel” will refer to the highly mineralized tissue which forms the outer layer of the tooth. The enamel layer is composed primarily of crystalline calcium phosphate (hydroxyapatite) along with water and some organic material. As used herein, the terms “pellicle” and “tooth pellicle” will refer to the thin film (typically ranging from about 1 μm to about 200 μm thick) derived from salivary glycoproteins which forms over the surface of the tooth crown.

By “pharmaceutically acceptable” is meant a material that can be administered to a subject without causing undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined.

The term “binding” refers to the interaction between a corresponding pair of molecules or portions thereof that exhibit mutual affinity or binding capacity, typically due to specific or non-specific binding or interaction, including, but not limited to, biochemical, physiological, and/or chemical interactions. By “specific binding” or “selective binding” is meant that the molecules, such as peptides, that are able to bind to or recognize a binding partner (or a limited number of binding partners) to a substantially higher degree than to other, similar biological entities. For example, the molecule binds preferentially to the target as compared to non-target. Selective binding to is generally characterized by at least a two-fold greater binding to a target, as compared to a non-target. A molecule can be characterized by, for example, 5-fold, 10-fold, 20-fold or more preferential binding to the target as compared to one or more non-targets.

By “contact” or “contacting” is meant to allow or promote a state of immediate proximity or physical association between at least two elements. For example, to contact a tooth surface with a peptide is to provide physical association between the tooth surface and the peptide.

As used herein, the term “enamel-like’ refers to a composite that has a similar microstructure, similar composition, and similar mechanical properties to tooth enamel.

The term “antibacterial”, as used herein, refers to various forms of components, including a sheet or powder, that inhibit the growth and/or kill populations of bacteria.

The term, “in vitro” refers to an artificial environment and to processes or reactions that occur within an artificial environment. In vitro environments include, but are not limited to, in solution or suspension, and cell cultures. The term “in vivo” refers to in or associated with an organism, such as an animal.

The term “effective amount,” as used herein, refers to an amount of an agent that is sufficient to elicit a desired biological and/or a pharmacologic response. In some forms, an “effective amount” or “therapeutically effective amount” means a quantity sufficient to alleviate or ameliorate one or more symptoms of a disorder, disease, or condition being treated. As will be appreciated by the skilled artisan, the effective amount of an agent, e.g., an isolated peptide, may vary depending on various factors, for example, the desired biological response, the site being targeted, and on the agent being used.

As used herein, the term “subject” means any individual, organism or entity. The subject can be a vertebrate, for example, a mammal (e.g., rat, rabbit, mouse, dog, cat, goat, pig, or horse). Thus, the subject can be a human. The term does not denote a particular age or sex. Thus, adult and newborn subjects, whether male or female, are intended to be covered. The subject may be healthy or suffering from or susceptible to a disease, disorder or condition. A patient refers to a subject afflicted with a disease or disorder. The term “patient” includes human and veterinary subjects.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevention” or “preventing” means to administer a composition to a subject or a system at risk for or having a predisposition for one or more symptoms caused by a disease or disorder to cause cessation of a particular symptom of the disease or disorder, a reduction or prevention of one or more symptoms of the disease or disorder, a reduction in the severity of the disease or disorder, the complete ablation of the disease or disorder, stabilization or delay of the development or progression of the disease or disorder.

As used herein, the terms “reduce” and “inhibit” mean to decrease an activity, response, condition, disease, or other biological parameter. This can include, but is not limited to, the complete ablation of the activity, response, condition, or disease. It is understood that this is typically in relation to a standard or expected value. The reduction or inhibition may be a 10, 20, 30, 40, 50, 60, 70, 80, 90, 100%, or any amount of reduction in between as compared to native or control levels. In some forms, inhibition or reduction is relative to a state prior to administration of one or more therapeutics. In some forms, inhibition or reduction is relative to a control that is not administered one or more therapeutics.

The term “peptide” refers to a class of compounds composed of amino acids chemically bound together. In general, the amino acids are chemically bound together via amide linkages (CONH); however, the amino acids can be bound together by other chemical bonds known in the art. For example, the amino acids can be bound by amine linkages. Peptide as used herein includes oligomers of amino acids and small and large peptides, including polypeptides. Thus, the terms “protein,” “peptide,” and “polypeptide” are used interchangeably herein. The protein, peptide, or polypeptide can be of any size, structure, or function. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “percent (%) sequence identity” describes the percentage of nucleotides or amino acids in a candidate sequence that are identical with the nucleotides or amino acids in a reference nucleic acid sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). In some forms, the default parameters can be used to determine the identity for the polynucleotides or polypeptides of the present disclosure.

In some forms, the % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Compositions

Disclosed are compositions containing one or more peptides that bind to oral cavity surfaces, such as tooth enamel. The peptides have anti-biofouling and mineralizing abilities, and as such, are useful in many aspects of oral care. For example, the peptides can prevent microorganism adhesion to oral cavity surfaces, protect teeth against an acid environment, and/or reduce, prevent or reverse enamel demineralization.

### A. Peptides

Disclosed are isolated peptides capable of binding to oral cavity surfaces either in vitro or in vivo. Preferably, the peptide is capable of binding to a tooth surface, such as enamel. In some forms, upon contact with an oral cavity (e.g., tooth surface) the isolated peptide binds to tooth enamel, reduces or prevents tooth decay or demineralization, reduces or prevents bacterial biofilm formation on a tooth surface, reduces or prevents dental plaque formation, reduces or prevents bacterial adhesion to a tooth surface, promotes (e.g., induces or increases) mineralization of a tooth surface, inhibits growth and/or proliferation of bacteria in the oral cavity, or combinations thereof. The ability to promote mineralization is based on the peptide's affinity for minerals such as calcium and phosphate ions in the oral cavity. For example, in some forms, upon coating or otherwise being applied to a tooth surface (e.g., enamel), the peptide attracts calcium and/or phosphate ions in the oral cavity (e.g., from saliva) to promote generation of new crystal deposits on the tooth surface.

In some forms, the peptide contains two or more domains. The domains can confer one or more distinct functions to the peptide. For example, in some forms, the peptide contains an antimicrobial domain and a dentotropic (tooth-binding) moiety or domain. In preferred forms, the peptide contains an antimicrobial domain and a phosphoserine moiety or domain (e.g., a diphosphoserine domain (DPS)) (remineralization enhancing domain). In some forms, the peptide is a multifunctional antimicrobial (AMP) with three parts: a non-specific AMP, P-113, a specific targeting domain, C8, and a remineralization enhancing domain. Preferably, the remineralization enhancing domain is positioned at the N-terminus of the peptide. More preferably, the antimicrobial domain is position at the C-terminus of the peptide, and has no moiety bound to it at its C terminus.

In some forms, the antimicrobial domain is or is derived from a naturally occurring protein. Preferably, the antimicrobial domain is or is derived from a histatin protein or a portion thereof. Histatins are a family of at least 12 small, histidine-rich, cationic peptides secreted into human saliva by salivary glands with significant in vitro antimicrobial activity, especially against fungi such as Candida. Histatins 1, 3, and 5 are the most abundant, and they have been credited with most of the anticandidal activity, histatin 5 being the most effective. Histatins 1 and 3 are encoded by HIS1 and HIS2 genes, respectively; histatin 5 is a proteolytic product of histatin 3, and all the other histatins are believed to arise from histatins 1 and 3 by proteolytic processing. Besides killing the wild-type C. albicans, histatins have been found to be effective in the in vitro killing of Candida species resistant to the commonly used antimycotics, fluconazole, and amphotericin B, as well as of C. neoformans. See Russell M W, et al., Chapter 5—Innate Humoral Defense Factors, Mucosal Immunology (Third Edition), Academic Press, 2005, pages 73-93.

Among all histatins, histatin 5 has the most potent fungicidal activity against pathogenic fungi, including Candida albicans and other medically important Candida species, such as Candida kefyr, Candida krusei, and Candida parapsilosis, as well as Cryptococcus neoformans and Aspergillus fumigatus (Puri S. and Edgerton M., Eukaryot Cell, 13(8):958-64 (2014)). Histatin 5 potentially inhibits almost 100% of the C. albicans cells from germinating into more-virulent hyphae. Inhibitory and bactericidal activities of histatins have also been shown against various bacteria, including S. mutans, P. gingivalis, A. actinomycetemcomitans, P. aeruginosa, E. coli, and St. aureus (Russel M W., et al.). Histatins as natural antimicrobial peptides show little or no toxicity toward mammalian cells and a low tendency to elicit resistance and thus have great potential to be developed into a novel class of antimicrobials, although few in vivo studies have been published.

The amino acid sequence of various histatins are known in the art. For example, the amino acid sequence of histatin 3 (from which histatin 5 is proteolytically processed), available from the Uniprot database under ID No. P15516, is MKFFVFALILALMLSMTGADSHAKRHHGYKRKFHEKHHSHRGYRSNYLYDN (SEQ ID NO:3). In preferred forms, the antimicrobial domain is or is derived from histatin 5 (e.g., human histatin 5). An exemplary amino acid sequence of human histatin 5 is DSHAKRHHGYKRKFHEKHHSHRGY (SEQ ID NO:1). Thus, in some forms, the antimicrobial domain has the amino acid sequence DSHAKRHHGYKRKFHEKHHSHRGY (SEQ ID NO:1).

In some forms, the antimicrobial domain is or contains a portion of histatin 5. For example, in some preferred forms, the antimicrobial domain has the amino acid sequence AKRHHGYKRKFH (SEQ ID NO:2).

In some forms, the antimicrobial domain includes an amino acid sequence having at least 80%, 85%, 90%, 91%, 92%, 93%, 94%, 95%, 96%, 97%, 98%, 99% or more sequence identity to the sequence provided in any of SEQ ID NOs:1-3. For example, in some preferred forms, the antimicrobial domain has an amino acid sequence having at least 90% sequence identity to DSHAKRHHGYKRKFHEKHHSHRGY (SEQ ID NO:1). In some preferred forms, the antimicrobial domain has an amino acid sequence having at least 90% sequence identity to AKRHHGYKRKFH (SEQ ID NO:2).

In preferred forms, the disclosed isolated peptide contains a phosphoserine moiety or domain. Typically, the phosphoserine domain includes one or more phosphorylated serines (Sp). For example, the phosphoserine domain can include 1, 2, 3, 4, 5 or more phosphorylated serines. In preferred forms, the phosphoserine domain includes two phosphorylated serines (referred to herein as a diphosphoserine domain (DPS)).

The antimicrobial domain and phosphoserine domain can be arranged in any orientation within the peptide. For example, the phosphoserine domain can be at the N- or C-terminus of the peptide. In some forms, the peptide conforms to the following architecture/structure:
NH₂[antimicrobial domain]-[phosphoserine domain]COOH; or
NH₂[phosphoserine domain]-[antimicrobial domain]COOH
   wherein NH₂ is the N-terminus of the peptide, and COOH is the C-terminus of the peptide. In some forms, the “-” used in the general architecture above indicates the presence of an optional linker. In some forms, the “-” used in the general architecture above does not indicate the presence of a linker, but is rather used to visually designate the start of one domain from the end of another. Preferably, the phosphoserine domain is positioned at the C-terminus of the peptide.

In preferred forms, the peptide includes the amino acid sequence AKRHHGYKRKFH-SpSp (SEQ ID NO:4), wherein each Sp independently represents a phosphorylated serine.

In some forms, the mineralization domain includes two phosphoserines (DPS), and/or 24 amino acids DSSDSSDSSDSSDSSDSSDSSDSS (8DSS) (SEQ ID NO: 11) or an amino acid sequence having at least 80%, 85%, 90%, 91%, 92%, 93%, 94%, 95%, 96%, 97%, 98%, 99% or more sequence identity to the sequence provided in SEQ ID NO: 11.

In some forms, the isolated peptides disclosed herein do not include a linker. For example, in some forms, the P-113-DPS peptide AKRHHGYKRKFH-SpSp (SEQ ID NO:4) does not include a linker between the antimicrobial domain and phosphoserine domain. In some forms, the P-113-SK peptide AKRHHGYKRKFH-SKHKGGKHKGGKHKG (SEQ ID NO:5) does not include a linker between the antimicrobial domain and surface binding (SK) domain.

In some forms, a linker is present between one or more domains within the isolated peptide (e.g., between an antimicrobial domain and a dentotropic domain (e.g., phosphoserine domain)). For example, in some forms, the P-113-DA peptide AKRHHGYKRKFH-D-3,4-dihydroxyphenethylamine (SEQ ID NO:6) includes a linker (e.g., Aspartic acid (D)) between the antimicrobial domain and dopamine (DA) domain. In some forms, the antimicrobial domain and phosphoserine domain are fused via any appropriate linker known in the art. Generally, such linkers have no specific biological activity other than to join or to preserve some minimum distance or other spatial relationship between the domains. The linker may be as simple as a covalent bond (e.g., a carbon-carbon bond, disulfide bond, carbon-heteroatom bond, etc.), or it may be a polymeric linker many atoms in length. The linker can be an organic molecule, group, polymer, or chemical moiety. In some forms, the linker is a carbon-nitrogen bond of an amide linkage. In some forms, the linker is a cyclic or acyclic, substituted or unsubstituted, branched or unbranched aliphatic or heteroaliphatic linker. The linker can be a single amino acid (e.g., Aspartic acid (D) or any other amino acid) or a plurality of amino acids. Linker sequences adopt a flexible extended conformation and do not exhibit a propensity for developing an ordered secondary structure. Preferably, the linker contains amino acids. Typical amino acids in flexible linkers include Gly (G), Asn (N) and Ser (S).

In some forms, the antimicrobial domain and phosphoserine domain forming the isolated peptide are fused via a linker that includes from about 1-20 amino acids, inclusive. In some forms, the linker includes from 1-5, 1-10, 1-15, or 1-20 amino acids.

The isolated peptide can be of any length or size, as long as it retains functionality (e.g., binding to tooth enamel, anti-biofouling and mineralizing abilities). In some forms, the peptide can have a length of up to 30 residues. For example, the peptide can have a length of about 6-30 residues, such as about 6-20 residues, about 10-15, or about 20-30 residues. In particular forms, the peptide has a length of 10, 11, 12, 13, 14, 15, or 20 residues. In some preferred forms, the peptide has a length of 12-14 residues.

Suitable peptides also include variants of the disclosed peptides, such as the peptide of SEQ ID NO:4, and modifications thereof retaining the same functional activity. For example, suitable peptides can include one or more point mutations or substitutions (e.g., 1, 2, 3, 4, 5 or more mutations) at any amino acid residue of SEQ ID NO:4. The one or more substitutions can be conservative or non-conservative. For example, the peptide AKRHHGYKRKFH-SpSp (SEQ ID NO:4), can be modified by substituting one or more of the non-polar amino acid residues (A, G), with another, similarly non-polar residue, such as I or L. Alanine scanning of peptides is useful for identifying amino acids that can be modified without reducing binding or other properties of the peptide.

The data in the present application demonstrate that about 2 μM mL⁻¹ of 8DSS-C8-P-113 effectively eliminates planktonic S. mutans without disrupting the oral normal flora (S. gordonii and S. sanguinis). Furthermore, at a concentration of about 8 μM mL⁻¹, the agent exhibits the ability to prevent S. mutans' adhesion. At a higher concentration of 16 μM mL⁻¹, 8DSS-C8-P-113 forms a hydrogel state, eradicating S. mutans' biofilm, adhering on the tooth surface against acid injury, and facilitating the repair of demineralized hard tissue. It is possible that 8DSS-C8-P-113 influences S. mutans by cell membrane perforation, interference with quorum sensing (QS), interference with glycolysis, DNA polymerase inhibition and protein modification inhibition, which were analysed by transcriptomic analysis. Molecular dynamics analyses revealed that 8DSS-C8-P-113 hydrogel has a strong affinity to hydroxyapatite (HAP) (100) surface. This affinity induces calcium and phosphate ions to bind to HAP (001) surface, leading to the formation of a needle-shaped remineralization crystal layer between the 8DSS-C8-P-113 hydrogel and the tooth surface. This multi-functional bioactive AMP 8DSS-C8-P-113 can be used in the disclosed methods to specifically target and kill pathogens and is effective in inducing in situ bio-mimic remineralization for the treatment of decayed teeth.

Thus, in some forms, the compositions includes 8DSS-C8-P-113 about 2 μM mL-1 which effectively eliminates planktonic S. mutans without disrupting S. gordonii and S. sanguinis.

In some forms, 8DSS-C8-P-113 decreases the amount of planktonic S. mutans by about 50% to about 100%.

In some forms, the compositions incudes 8DSS-C8-P-113 at about 8 μM mL-1, which exhibits the ability to prevent S. mutans' adhesion by interfering with glycolysis, thereby reducing virulence. In some forms, the 8DSS-C8-P-113 can decrease the amount of S. mutans' adhesion by about 50% to about 100%.

In some forms, when used at a concentration of about 16 μM mL⁻¹, 8DSS-C8-P-113 eradicates S. mutans' biofilm. While not being bound by theory, it is thought 8DSS-C8-P-113 eradicates S. mutans' biofilm by interfering with QS and it forms a hydrogel state on the tooth surface against acid injury. In some embodiments, 8DSS-C8-P-113 eradicates S. mutans' biofilm by about 20% to about 100%.

In other forms, at about 16 μM mL-1, 8DSS-C8-P-113 induces the binding of calcium and phosphate ions to the HAP (001) surface, resulting in the formation of a needle-shaped remineralization crystal layer between the 8DSS-C8-P-113 hydrogel and the tooth surface. This process mimics natural bio-mineralization and promotes tooth remineralization. In summary, this bioactive AMP 8DSS-C8-P-113 exhibits multiple functions, including specifically targeted killing of S. mutans, reduction of S. mutans' virulence, protecting tooth surface against acid injury, and induction of in situ bio-mimic remineralization. These properties make it a useful agent for the treatment of dental caries.

Suitable peptides also include variants of the disclosed peptides. The term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical, but in all cases retain the same functional activity or mechanism of action. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and cofactors. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity. The following hydrophilicity values can be assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, and size. Exemplary substitutions that take various of the foregoing characteristics into consideration are well known to those of skill in the art and include (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of the peptides can include variants having about 50%, 60%, 70%, 80%, 90%, 95%, 96%, 97%, 98%, 99%, or more sequence identity to the peptide of interest. The term “conservative amino acid substitution”, is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

### B. Peptide Modifications

The disclosed peptides may be modified in various ways. In some forms, the modification(s) may render the peptides more stable (e.g., resistant to degradation in vivo) or confer other desirable characteristics as will be appreciated by one skilled in the art. Such modifications include, without limitation, chemical modification, N terminus modification, C terminus modification, peptide bond modification, backbone modifications, residue modification, D-amino acids, or non-natural amino acids or others. In some forms, one or more modifications may be used simultaneously. In preferred forms, the peptides are stabilized against proteolysis. For example, the stability and activity of peptides can be improved by protecting some of the peptide bonds with N-methylation or C-methylation. It is believed that modifications, such as amidation, also enhance the stability of peptides to peptidases.

Modifications to the peptides generally should leave them functional. It is understood that there are numerous amino acid analogs which can be incorporated into the peptides. For example, there are numerous D amino acids or other non-natural amino acids which can be used. The opposite stereoisomers of naturally occurring peptides are disclosed, as well as the stereo isomers of peptide analogs. Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

Either or both ends of a given linear peptide can be modified. For example, the peptides can be acetylated and/or amidated.

The peptides may contain naturally occurring α-amino acid residues, non-naturally occurring α-amino acid residues, and combinations thereof. The D-enantiomer (“D-α-amino acid”) of residues may also be used. Incorporation of artificial amino acids such as beta or gamma amino acids and those containing non-natural side chains, and/or other similar monomers such as hydroxyacids are also contemplated, with the effect that the corresponding component is peptide-like in this respect.

Non-naturally occurring amino acids are not found or have not been found in nature, but they can by synthesized and incorporated into a peptide chain. Non-limiting examples of suitable non-natural amino acids (in L- or D-configuration) are azidoalanine, azidohomoalanine, 2-amino-5-hexynoic acid, norleucine, azidonorleucine, L-a-aminobutyric acid, 3-(1-naphthyl)-alanine, 3-(2-naphthyl)-alanine, p-ethynyl-phenylalanine, m-ethynyl-phenylalanine, p-ethynyl- phenylalanine, p-bromophenylalanine, p-idiophenylalanine, p-azidophenylalanine, and 3-(6-chloroindolyl) alanin.

In some forms, peptide bonds (—CO—NH—) within the peptide may be substituted, for example, by N-methylated bonds (—N(CH₃)—CO—), ester bonds (—C(R)H—C-0-0-C(R)—N—), ketomethylen bonds (—CO—CH₂—), CC-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the normal side chain, naturally presented on the carbon atom. These modifications can occur at any of the bonds along the peptide chain and even at several (e.g., 2, 3, 4 or more) at the same time.

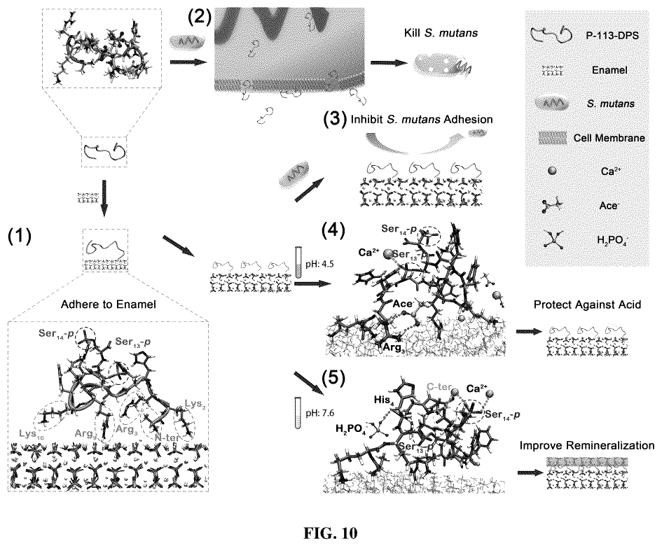

The peptides can be utilized in a linear form, although it will be appreciated that in cases where cyclization does not severely interfere with peptide characteristics, cyclic forms of the peptides can also be used. For example, in some forms, an isolated P-113-DPS peptide (AKRHHGYKRKFH-SpSp; SEQ ID NO:4) can be in a cyclic form because of intramolecular bonds, and its conformation can change upon adsorption onto a tooth surface (see FIG. 10). As used herein in reference to a peptide, the term “cyclic” means a structure including an intramolecular bond between two non-adjacent amino acids or amino acid analogues. The cyclization can be effected through a covalent or non-covalent bond. A preferred method of cyclization is through formation of a disulfide bond between the side-chains of non-adjacent amino acids or amino acid analogs. A peptide also can cyclize, for example, via a lactam bond, which can utilize a side-chain group of one amino acid or analog thereof to form a covalent attachment to the N-terminal amine of the amino-terminal residue. Cyclization additionally can be effected, for example, through the formation of a lysinonorleucine bond between lysine (Lys) and leucine (Leu) residues or a dityrosine bond between two tyrosine (Tyr) residues. The skilled person understands that these and other bonds can be included in a cyclic peptide.

Peptidomimetics may optionally be used to inhibit degradation of the peptides by enzymatic or other degradative processes. The peptidomimetics can be produced by organic synthetic techniques. Non-limiting examples of suitable peptidomimetics include D amino acids of the corresponding L amino acids. D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides as long as activity is preserved.

### C. FAP-LPAA

In certain embodiments, the FAP-LPAA sheet has comparable physicochemical property including, for example, mechanical properties to dental enamel and has excellent biocompatibility and antimicrobial properties, which can be indicated by dyeing bacteria to determine that FAP-LPAA killed the bacterial cells. Elastic modulus and hardness of the material can be measured by nanoindentation. Wear depth of the material can be measured by micro-scale abrasion tests. The biocompatibility of the material can be related to the safety of the materials, and the measurement of biocompatibility can indicate if the materials have potential toxicity to cells of the subject. The biocompatibility of the material is measured by cell counting, such as, for example, using the Cell Counting Kit-8 (CCK-8) and dyeing of cytoskeleton and cell nucleus. The FAP-LPAA can be administered to a subject, specifically on one or more teeth of a subject. The subject can have dental caries, tooth decay, enamel hypoplasia, tooth lesions, or bone-filling material. The FAP-LPAA can be applied directly to the tooth, bone, or enamel and bonded to said tooth, enamel, or bone via dental adhesives.

### D. Formulations and Devices

The disclosed peptides can be combined with a pharmaceutically acceptable carrier or excipient to form a pharmacological composition. For example, pharmaceutical compositions including an effective amount of the peptide or a plurality of copies of the peptide and a pharmaceutically acceptable excipient are provided. Typically, the excipient is suitable for administration to oral cavity. Pharmaceutically acceptable carriers can contain one or more physiologically acceptable compound(s) that act, for example, to stabilize the composition or to increase or decrease the absorption of the peptide(s). Suitable carriers, diluents and their formulations are described in Remington: The Science and Practice of Pharmacy (19th ed.) ed. A. R. Gennaro, Mack Publishing Company, Easton, PA 1995. Those of skill in the art can readily determine the various parameters for preparing and formulating the compositions without resort to undue experimentation. Physiologically acceptable compounds can include, for example, carbohydrates, such as glucose, sucrose, or dextrans, antioxidants, such as ascorbic acid or glutathione, chelating agents, low molecular weight proteins, protection enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disintegrants, lubricants, suspending agents, and the like. Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain forms, the excipients are sterile and generally free of undesirable matter. These compositions can be sterilized by conventional, well-known sterilization techniques. For various oral dosage form excipients such as tablets and capsules sterility is not required.

In some forms, the peptides are formulated into pharmaceutical compositions for local administration. In preferred forms, pharmaceutical compositions of the peptides may be for topical administration, such as, to the oral cavity or mucosa, including tooth surfaces.

The peptides may be used in oral care products, which may have any suitable physical form, such as powder, paste, gel, liquid, ointment, or tablet. Exemplary oral care products include, but are not limited to, toothpaste, dental cream, gel or tooth powder, mouthwash, breath freshener, oral sprays, rinses, gargles, gums, tablets, oral lozenges, tooth whitening strips or solutions, cleaning solutions, toothpicks, toothbrushes, dental floss, varnish, and restorative materials, including, but not limited to, composite resin. Typically, the oral care products contain an effective amount of the peptides in an orally acceptable carrier. An effective amount of a peptide for use in an oral care product may vary depending on the type of product. Typically, the effective amount of the peptides is a proportion from about 0.001% to about 90% by weight of the total product composition. The oral care product may contain one type of peptide or a mixture of different peptides (e.g., of different sequence and/or length).

Components of an orally acceptable carrier are described in U.S. Pat. Nos. 6,740,311; 6,706,256; and 6,264,925; all of which are incorporated herein by reference. For example, in addition to the peptides, the oral care products may contain one or more of the following: abrasives, surfactants, chelating agents, fluoride sources, thickening agents, buffering agents, solvents, humectants, carriers, bulking agents, and additional oral benefit agents, as given above. Oral care products may be prepared using standard techniques that are well known in the art. If a composition contains more than one phase, typically, the different phases are prepared separately, with material of similar phase partitioning being added in any order. The two phases are combined using vigorous mixing to form the multiphase system (e.g., an emulsion or dispersion).

In some forms, the peptides are incorporated into oral care formulations, e.g., a prescription or over the counter product for use in a home, for travel, at work, in a dental office, at a hospital, etc. Such formulations include, but are not limited to toothpastes, mouthwashes, tooth whitening strips or solutions, cleaning solutions, dental floss, toothpicks, toothbrushes, oral sprays, oral lozenges, and the like.

The formulation of such oral care products is well known to those of skill, and the disclosed peptides can simply be added to such formulations in an effective dose (e.g., a prophylactic dose to inhibit dental carie formation, etc.). For example, toothpaste formulations are well known to those of skill in the art. Typically such formulations are mixtures of abrasives and surfactants; anticaries agents, such as fluoride; tartar control ingredients, such as tetrasodium pyrophosphate and methyl vinyl ether/maleic anhydride copolymer; pH buffers; humectants, to prevent dry-out and increase the pleasant mouth feel; and binders, to provide consistency and shape. Binders keep the solid phase properly suspended in the liquid phase to prevent separation of the liquid phase out of the toothpaste. They also provide body to the dentifrice, especially after extrusion from the tube onto the toothbrush.

In preparing toothpastes or gels, a thickening material can be added to provide a desirable consistency of the composition, to provide desirable active release characteristics upon use, to provide shelf stability, and to provide stability of the composition, etc. Exemplary thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents in an amount from about 0.1% to about 15%, preferably from about 2% to about 10%, more preferably from about 4% to about 8%, by weight of the total toothpaste or gel composition, can be used. Higher concentrations can be used for chewing gums, lozenges (including breath mints), sachets, non-abrasive gels and subgingival gels.

Mouthwash formulations are also well known to those of skill in the art. For example, mouthwashes containing sodium fluoride are disclosed in U.S. Pat. Nos. 2,913,373, 3,975,514, and 4,548,809, and in US Patent Publications US 2003/0124068, US 2007/0154410. Mouthwashes containing various alkali metal compounds are also known: sodium benzoate (WO 94/09752); alkali metal hypohalite; chlorine dioxide (CN 1222345); alkali metal phosphate (US 2001/0002252, US 2003/0007937); hydrogen sulfate/carbonate (JP 8113519); cetylpyridium chloride (CPC) (see, e.g., U.S. Pat. Nos. 6,117,417, 5,948,390, and JP 2004051511). Mouthwashes containing higher alcohol (see, e.g., US 2002/0064505, US 2003/0175216); hydrogen peroxide (see, e.g., CN 1385145); CO₂ gas bubbles (see, e.g., JP 1275521 and JP 2157215) are also known. In some forms, these and other mouthwash formulations can further include one or more of the disclosed peptides.

Oral sprays, oral rinses, oral cleaning solutions, oral lozenges, gums, tablets, gargles, dental creams, and devices or instruments such as toothbrushes, toothpicks, dental floss, tooth whitening strips, and the like are also well known to those of skill in the art and can readily be adapted to incorporate the disclosed peptides. For example, the peptides can be incorporated onto the bristles of a toothbrush, or as a coating on dental floss or toothpicks. Suitable lozenge and chewing gum components are disclosed in U.S. Pat. No. 4,083,955.

In some forms, the oral care compositions include a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, can be present in a concentration from about 0% to about 70%, preferably from about 5% to about 25%, by weight of the compositions. Suitable humectants include edible polyhydric alcohols such as glycerin, sorbitol, xylitol, butylene glycol, polyethylene glycol, and propylene glycol, especially sorbitol and glycerin.

Flavoring and sweetening agents can also be included in the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition. Sweetening agents which can be used include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A composition preferably contains from about 0.1% to about 10% of these agents, preferably from about 0.1% to about 1%, by weight of the composition.

In addition, coolants, salivating agents, warming agents, and numbing agents can be used as optional ingredients in compositions. These agents are present in the compositions at a level of from about 0.001% to about 10%, preferably from about 0.1% to about 1%, by weight of the composition. The coolant can be any of a wide variety of materials. Included among such materials are carboxamides, menthol, ketals, diols, and mixtures thereof.

The pH of the compositions can be adjusted through the use of buffering agents. In some forms, buffering agents can be used to adjust the pH of the compositions to a range of about 4.5 to about 9.5. Buffering agents include monosodium phosphate, trisodium phosphate, sodium hydroxide, sodium carbonate, sodium acid pyrophosphate, citric acid, and sodium citrate.

The foregoing oral care formulations and/or devices are meant to be illustrative and not limiting. Using the teaching provided herein, the peptides of the present invention can readily be incorporated into other products.

### III. Methods of Manufacture

Methods of preparing the disclosed peptides and compositions thereof are described. In some forms, the peptides can be obtained commercially, such as from a vendor which provides custom peptide synthesis services. In some forms, peptides having a desired sequence can be synthesized or produced recombinantly. Thus, methods of making the peptides using known techniques are provided.

In some forms, the peptides can be produced by recombinant means (e.g., in bacteria, yeast, fungi, insect, vertebrate or mammalian cells) by methods well known to those skilled in the art. Generally this involves creating a DNA sequence that encodes the desired peptide, placing the DNA in an expression cassette under the control of a particular promoter, expressing the peptide in a host, isolating and/or purifying the expressed peptide and, if required, renaturing the peptide.

DNA encoding the peptide(s) can be prepared by any suitable method as described above, including, for example, cloning and restriction of appropriate sequences or direct chemical synthesis. The nucleic acid can be easily ligated into an appropriate vector containing appropriate expression control sequences (e.g. promoter, enhancer, etc.), and, optionally, containing one or more selectable markers (e.g. antibiotic resistance genes). The nucleic acid sequences encoding the peptides can be expressed in a variety of host cells, including, but not limited to, E. coli, other bacterial hosts, yeast, fungus, and various higher eukaryotic cells such as insect cells (e.g. SF3), the COS, CHO and HeLa cells lines, and myeloma cell lines. Once expressed, the recombinant peptide(s) can be purified according to standard procedures of the art, including ammonium sulfate precipitation, affinity columns, column chromatography, gel electrophoresis and the like.

In certain forms, the peptides are chemically synthesized by any of a number of fluid or solid phase peptide synthesis techniques known to those of skill in the art. For example, standard FMOC synthesis is described in the literature (e.g., solid phase peptide synthesis, see E. Atherton, RC Sheppard, Oxford University press (1989), or liquid phase synthesis (where peptides are assembled using a mixed strategy by BOC chemistry and fragment condensation). Solid phase synthesis in which the C-terminal amino acid of the sequence is attached to an insoluble support followed by sequential addition of the remaining amino acids in the sequence is a preferred method for the chemical synthesis of the peptides. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc, 85: 2149-2156, and Stewart et al. (1984) Solid Phase Peptide Synthesis, 2nd ed. Pierce Chem. Co., Rockford, 111.

Such methods include bench scale solid phase synthesis and automated peptide synthesis in any one of the many commercially available peptide synthesizers. Solid phase synthesis is commonly used and various commercial synthesizers are available, for example automated synthesizers by Applied Biosystems Inc., Foster City, CA; Beckman; MultiSyntech, Bochum, Germany etc. Solution phase synthetic methods may also be used, although this can be less convenient. Functional groups for conjugating the peptide to small molecules, label moieties, peptides, or proteins may be introduced into the molecule during chemical synthesis. In addition, small molecules and label moieties/reporter units may be attached during the synthetic process. Preferably, introduction of the functional groups and conjugation to other molecules minimally affects the structure and function of the subject peptide.

The peptides can be produced by stepwise synthesis or by synthesis of a series of fragments that can be coupled by well-known techniques. Chemical synthesis typically starts from the C-terminus, to which amino acids are sequentially added using either a Rink amide resin (resulting in an —NH₂ group at the C-terminus of the peptide after cleavage from the resin), or a Wang resin (resulting in an —OH group at the C-terminus). Accordingly, peptides having a C-terminal moiety that may be selected from the group consisting of —H, —OH, —COOH, —CONH₂, and —NH₂ are contemplated for use.

Standard Fmoc (9-florenylmethoxycarbonyl) derivatives include Fmoc-Asp(OtBu)-OH, Fmoc-Arg(Pbf)-OH, and Fmoc-Ala-OH. Couplings are mediated with DIC (diisopropylcarbodiimide)/6-Cl-HOBT (6-chloro-1-hydroxybenzotriazole). In some embodiments, the last four residues of the peptide require one or more recoupling procedures. In particular, the final Fmoc-Arg(Pbf)-OH coupling can require recoupling. For example, a second or third recoupling can be carried out to complete the peptide using stronger activation chemistry such as DIC/HOAT (1-hydroxy-7-azabenzotriazole) or HATU (1-[bis(dimethylamino)methylene]-1H-1,2,3-triazolo[4,5-b]pyridinium 3-oxid hexafluorophosphate)/NMM (N-methylmorpholine).

Acidolytic cleavage of the peptide can be carried out with the use of carbocation scavengers (thioanisole, anisole and H₂O). Optimization can be achieved by varying the ratio of the components of the cleavage mixture. An exemplary cleavage mixture ratio is 90:2.5:2.5:5 (TFA-thioanisole-anisole-H₂O). The reaction can be carried out for 4 hours at room temperature. In some forms, the removal of residual impurities is carried out by wash steps. For example, trifluoroacetic acid (TFA) and organic impurities can be eliminated by precipitation and repeated washes with cold diethyl ether and methyl t-butyl ether (MTBE).

Peptides produced using the disclosed methods can be purified using high pressure liquid chromatography (HPLC). Suitable solvents for dissolving the peptides include neat TFA. Typically, the peptides remain soluble at TFA concentrations of 0.5% to 8% and can be loaded onto reverse phase (RP)-HPLC columns for salt exchange. Exemplary salt exchange methods use 3-4 column volumes of acidic buffer to wash away the TFA counter ion due to its stronger acidity coefficient. Buffers suitable for use in washing away the TFA counter ion include 0.1% HCl in H₂O.

Following removal of TFA, peptides can be eluted with a step gradient. Exemplary elution buffers include 30% acetonitrile (MeCN) vs. 0.1% HCl in H₂O. For acetate exchange, peptides can be loaded from the same diluted TFA solution, washed with 3-4 column volumes of 1% acetic acid (AcOH) in H₂O, followed by 2 column volumes of 0.1 M NH₄OAc in H₂O, pH 4.4. In some embodiments, the column is washed again with 3-4 column volumes of 1% AcOH in H₂O.

Analytical HPLC can be carried out to assess the purity and homogeneity of peptides. An exemplary HPLC column for use in analytical HPLC is a PHENOMENEX® JUPITER® column. A step gradient can be used to separate the peptide composition. In some embodiments, the gradient is from 1%-40% MeCN vs 0.05% TFA in H₂O. The change in gradient can be achieved over 20 minutes using a flow rate of 1 ml/min. Peptides can be detected using UV detection at 215 nm.

Where the peptides or compositions thereof are required to be sterilized or otherwise processed for the removal of undesirable contaminants and/or micro-organisms, filtration can be used. Filtration can be achieved using any system or procedures known in the art. In some forms, filtration removes contaminants or prevents the growth or presence of microorganisms. Exemplary microorganisms and contaminants that can be removed include bacteria, cells, protozoa, viruses, fungi, and combinations thereof. In some forms, the step of filtration is carried out to remove aggregated or oligomerized peptides. For example, solutions of the peptides can be filtered to remove oligomers on the basis of size.

### IV. Methods of Use

Disclosed herein are various methods related to the disclosed peptides and compositions and their use. The peptides and compositions thereof can be used in therapeutic and/or prophylactic applications. For example, disclosed are methods of using the peptides and compositions thereof to reduce or prevent tooth decay, repair tooth decay, reduce or prevent enamel demineralization, promote enamel mineralization, or combinations thereof.

In some forms, the antimicrobial peptide composition can be applied to the enamel and/or dentin of a tooth of a subject. The antimicrobial peptide can be bonded to enamel and/or dentin surface by dental adhesives.

In some forms, the subject is infected with Streptococcus mutans, or any other bacteria known to be involved dental caries, such as Actinomyces spp., and Atopobium spp.

In some forms, the FAP-LPAA composition can be used to treat bacterial infections or prevent bacterial infections, particularly of the tooth or gums.

In some forms, the compositions of the subject invention can be used to reduce tooth decay caused by bacteria.

The peptides can be incorporated into or onto an oral care composition or device. An “oral care composition” is any composition that is suitable for administration or application to the oral cavity of a human or animal subject for enhancing the health, hygiene or appearance of the subject, for example, providing benefits such as: the prevention or treatment of a condition or disorder of the teeth, gums, mucosa or other hard or soft tissue of the oral cavity; the provision of sensory, decorative or cosmetic benefits; and combinations thereof. Typically, the oral care composition, in the ordinary course of usage, is not intentionally swallowed for purposes of systemic administration, but is rather retained in the oral cavity for a time sufficient to contact substantially all of the dental surfaces and/or oral tissues for purposes of oral activity. The oral composition may be a single phase oral composition or may be a combination of two or more oral compositions. Exemplary oral care compositions or devices include, without limitation, mouthwashes, toothpastes, dental creams, oral sprays, rinses, gargles, gums, tablets, oral lozenges, dental floss, tooth whitening strips or solutions, cleaning solutions, toothpicks, and toothbrushes.

For example, in some forms, the peptides can be incorporated into toothpaste or mouthwash to reduce or prevent colonization or recolonization of the oral cavity and thereby reduce the incidence and/or degree of dental carie formation. In various forms, the peptides can be incorporated into dental floss for similar purposes or can be provided in swabs that are used to swab the teeth and oral mucosa.

In some forms, the peptides and compositions thereof are administered locally. In some forms, the peptides and compositions thereof are administered topically, e.g., applied to the oral cavity.

The peptides and compositions thereof can be used therapeutically and/or prophylactically. For example, in some forms, subject administered the peptides and compositions thereof suffers from an infection or is at risk for infection (e.g., pathogenic bacterial infection in the mouth). In some forms, the subject administered the peptides and compositions thereof suffers from dental caries or is at risk for developing dental caries.

The peptides and compositions thereof can be used in methods to treat and/or prevent dental caries or other pathologies of the teeth or oral mucosa. In some forms, the peptides and compositions thereof are used in methods to repair tooth decay (e.g., promote mineralization of a tooth surface, such as enamel). In some forms, the peptides and compositions thereof are used in methods to reduce or prevent tooth surface (e.g., enamel) demineralization. In some forms, the peptides and compositions thereof are used in methods to reduce or prevent adhesion of microorganisms (e.g., bacteria) to the tooth surface. In some forms, the peptides and compositions thereof are used in methods to reduce or prevent formation of biofilms on the tooth surface. In some forms, the peptides and compositions thereof are used in methods to reduce or prevent dental plaque formation. In some forms, the peptides and compositions thereof are used in methods to inhibit growth and/or proliferation bacteria in the oral cavity (e.g., caries-causing bacteria such as S. mutans). In some forms, the peptides and compositions thereof are used in methods to kill bacteria in the oral cavity (e.g., caries-causing bacteria such as S. mutans). The methods can also include cleaning and polishing teeth and reducing the incidence of stain, plaque, gingivitis and calculus on dental enamel. In certain forms, the method includes a combination of the foregoing.

It is understood that any of the foregoing methods can include administering or applying the peptides and compositions thereof to the subject though not explicitly recited. Thus, an exemplary method involves applying a disclosed oral care composition or device containing the peptides to the teeth and/or oral cavity of a subject. For example, a composition containing the peptides can be brushed, sprayed, or otherwise coated onto the teeth. In some forms, a composition containing the peptides is applied to the oral cavity by gargling, rinsing or shaking.

In some forms, the methods involve contacting a subject's dental enamel surfaces and/or oral mucosa with the oral care compositions. This can be accomplished by brushing, rinsing (e.g., with a slurry or mouthwash or rinse). Other methods include contacting a topical oral gel, paste, mouthspray, or other form with the subject's teeth and/or oral mucosa.

In some preferred forms, a method of reducing or preventing and/or repairing tooth decay in a subject in need thereof includes contacting the subject's teeth and/or oral cavity with a peptide having the sequence AKRHHGYKRKFH-SpSp (SEQ ID NO:4) or a sequence having at least 90% sequence identity to AKRHHGYKRKFH-SpSp (SEQ ID NO:4). In some preferred forms, a method of reducing or preventing enamel demineralization and/or promoting enamel mineralization in a subject in need thereof includes contacting the subject's teeth and/or oral cavity with a peptide having the sequence AKRHHGYKRKFH-SpSp (SEQ ID NO:4) or a sequence having at least 90% sequence identity to AKRHHGYKRKFH-SpSp (SEQ ID NO:4).

In some preferred forms, a method of reducing or preventing and/or repairing tooth decay in a subject in need thereof includes contacting the subject's teeth and/or oral cavity with a peptide having the sequence of SEQ ID NO:12, SEQ ID NO:8, or SEQ ID NO:10 or a sequence having at least 90% sequence identity to having the sequence of SEQ ID NO:12, SEQ ID NO:8, or SEQ ID NO:10. In some preferred forms, a method of reducing or preventing enamel demineralization and/or promoting enamel mineralization in a subject in need thereof includes contacting the subject's teeth and/or oral cavity with a peptide having the sequence of having the sequence of SEQ ID NO:6, SEQ ID NO:8, or SEQ ID NO:10) or a sequence having at least 90% sequence identity to having the sequence of SEQ ID NO:12, SEQ ID NO:8, or SEQ ID NO:10.

The subject may be any human or lower animal. For example, the subject can be a household pet or other domestic animals, or animals kept in captivity. For example, a method of use may include brushing a dog's teeth with one of the disclosed toothpastes, toothbrushes, or other compositions or devices. Another example would include the rinsing of a cat's mouth with an oral composition for a sufficient amount of time to see a benefit. Pet care products such as chews and toys may be formulated to contain the oral care compositions. The composition including the peptides can be incorporated into a relatively supple but strong and durable material such as rawhide, ropes made from natural or synthetic fibers, and polymeric articles made from nylon, polyester or thermoplastic polyurethane. As the animal chews, licks or gnaws the product, the incorporated active elements are released into the animal's oral cavity into a salivary medium, comparable to an effective brushing or rinsing.

Preferably, the subject is a human.

### Effective Amounts

Typically, the methods involve administering or applying an effective amount of the peptides or compositions thereof. For example, in some embodiments, the peptide compositions are administered to a subject in an effective amount for treatment and/or prevention of a disease, disorder or condition (e.g., dental caries or other pathologies of the teeth or oral mucosa).

The effective amount can be a dosage sufficient to treat, inhibit, or alleviate one or more symptoms of a disorder, disorder or condition being treated or to otherwise provide a desired pharmacologic and/or physiologic effect. The effective amount of the peptide compositions will vary from subject to subject, and can depend on the species, age, weight and general condition of the subject, the severity of the disorder being treated, and its mode of administration. Thus, it is not possible to specify an exact amount for every therapeutic composition. However, an appropriate amount can be determined by one of ordinary skill in the art using only routine experimentation given the teachings herein. For example, effective dosages and schedules for administering the therapeutics may be determined empirically, and making such determinations is within the skill in the art. The dosage ranges for the administration of the compositions are those large enough to effect one or more desired responses.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage can depend upon the age, condition, and sex of the subject, the desired therapeutic effect, on the route of administration, and on the duration of the treatment desired. It will also be appreciated that the effective dosage of the composition used for treatment may increase or decrease over the course of a particular treatment.

The concentration of peptides in the compositions or the total amounts can vary widely, and will be selected primarily based on activity of the particular peptide(s) and/or composition thereof, particular mode of administration selected, and the subject's needs. Typical dosages range from about 1 μM mol⁻¹ to 100 μM mol⁻¹. For example, in some forms, the concentration of peptide(s) is in the range of about 5-10 μM mol⁻¹, 10-50 μM mol⁻¹, 20-60 μM mol⁻¹, 30-70 μM mol⁻¹, 40-80 μM mol⁻¹, or 50-100 μM mol⁻¹. In some forms, the concentration of peptide(s) is about 5, 10, 20, 30, 40, 50, 60, 70, 80, 90, or 100 μM mol⁻¹. In particular forms, the concentration of peptide(s) is about 8 μM mol⁻¹ or 64 μM mol⁻¹.

In some forms, the peptides are in an amount effective to reduce or prevent tooth decay, reduce or prevent bacterial biofilm formation, reduce or prevent dental plaque formation, reduce or prevent bacterial adhesion to tooth surfaces, inhibit growth and/or proliferation of oral bacteria, induce or increase enamel mineralization, or combinations thereof.

The mouth is colonized by 200 to 300 bacterial species, but only a limited number of these species participate in dental decay (caries) or periodontal disease. The tooth surface normally loses some tooth mineral from the action of the acid formed by plaque bacteria after ingestion of foods containing fermentable carbohydrates. This mineral is normally replenished by the saliva between meals. However, when fermentable foods are eaten frequently, the low pH in the plaque is sustained and a net loss of mineral from the tooth occurs. This low pH selects for aciduric organisms, such as S. mutans and lactobacilli, which store polysaccharide and continue to secrete acid long after the food has been swallowed. Reports indicate that bacterial species such as species of the genera Veillonella, Lactobacillus, Bifidobacterium, and Propionibacterium, low-pH non-S. mutans streptococci, Actinomyces spp., and Atopobium spp., likely play important roles in caries progression (Aas J A., et al., J Clin Microbiol., 46(4):1407-1417 (2008)).

Thus, in some forms, the bacteria contained in the biofilm and/or dental plaque, the bacteria whose adhesion is reduced or prevented, and/or whose growth and/or proliferation is inhibited is bacteria present in the oral cavity such as species of the genera Streptococcus, Veillonella, Lactobacillus, Bifidobacterium, and Propionibacterium. In some forms, the bacteria contained in the biofilm and/or dental plaque, the bacteria whose adhesion is reduced or prevented, and/or whose growth and/or proliferation is inhibited is gram positive bacteria.

In some forms, the bacteria contained in the biofilm and/or dental plaque, the bacteria whose adhesion is reduced or prevented, and/or whose growth and/or proliferation is inhibited is acidophilic bacteria or acid-producing bacteria.

In any of the foregoing, the bacteria can be selected from S. mutans, S. faecalis, S. parasanguinis, S. salivarius, S. sobrinus, L. acidophilus, L. casei, Actinomyces spp., Atopobium spp., Corynebacterium sp., Leptotrichia sp., A. gerencseriae, and combinations thereof.

### Timing of Administration and Regimens

Dosages and timing of administration can vary. Guidance can be found in the literature for appropriate dosages for given classes of pharmaceutical products. Persons of ordinary skill can determine optimum dosages, dosing methodologies and repetition rates. Optimum dosages may vary depending on the relative potency of individual therapeutics, and can generally be estimated based on EC₅₀s, minimal bactericidal concentrations (MBC), and/or minimum inhibitory concentrations (MIC) found to be effective in in vitro and in vivo animal models.

Administration of the peptides and compositions thereof can be continued for an amount of time sufficient to achieve one or more desired goals (e.g., therapeutic or prophylactic goals). Treatment can be continued for a desired period of time, and the progression of treatment can be monitored using any suitable means known in the art. In some forms, administration is carried out every day of treatment (e.g., multiple times a day), or every week, or every fraction of a week. In some forms, administration regimens are carried out over the course of up to two, three, four or five days, weeks, or months, or for up to 6 months, or for more than 6 months, for example, up to one or two years.

Single or multiple administrations of the compositions may be administered depending on the dosage and frequency as required and tolerated by the subject. In any event, the composition should provide a sufficient quantity of the peptides to effect the desired outcome. In some forms, the multiple doses of the compositions are administered before an improvement in disease condition is evident. For example, in some forms, the subject receives the composition, over a period of 1, 2, 3, 4, 5, 6 7, 14, 21, 28, 35, or 48 days or weeks before an improvement in the disease or condition is evident.

In some forms, the composition is administered or applied for a time of from about 30 seconds to about 30 minutes, for example about 30 seconds, about 1, 2, 3, 4, 5, 6, 7, 8, 9, 10, 11, 12, 13, 14, 15, 16, 17, 18, 19, 20, 25, or 30 minutes. In some forms the composition is administered or applied one or more times per day, e.g., 1, 2, 3 or more times per day.

### V. Kits

The disclosed reagents, materials, and compositions as well as other materials can be packaged together in any suitable combination as a kit useful for performing, or aiding in the performance of, the disclosed methods. It is useful if the components in a given kit are designed and adapted for use together in the disclosed method.

In some forms, the kits can include, for example, a composition or device containing one or more disclosed peptides. The kits typically include a container containing one or more of the active agents (e.g., the disclosed peptides) described herein. In certain forms, the active agent(s) can be provided in a unit dosage formulation (e.g., suppository, tablet, cap let, patch, etc.) and/or may be optionally combined with one or more pharmaceutically acceptable excipients. In particular forms, the kits include one or more oral care compositions or devices such as a toothpaste, mouthwash, toothpick, dental floss, gum, gel, or combinations thereof.

Preferably, the kits include instructional material. The instructional material can include a publication, a recording, a diagram, or any other medium of expression which can be used to communicate the usefulness of the compositions and methods of the kit.

For example, the instructional material may provide instructions for methods using the kit components, such as performing oral rinses, oral gargles, teeth cleaning, teeth whitening, and the like.

The present invention will be further understood by reference to the following non-limiting examples.

### EXAMPLES

The following example is put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### Example 1: P-113-DPS Exhibits Antibacterial and Remineralizing Properties

### Materials and Methods

### Synthesis of Bioactive Peptides

Five bioactive peptides including P-113 (AKRHHGYKRKFH; SEQ ID NO:2), P-113-DPS (AKRHHGYKRKFH-SpSp; SEQ ID NO:4), P-113-DA (AKRHHGYKRKFH-D-3,4-dihydroxyphenethylamine; SEQ ID NO:6), P-113-SK (AKRHHGYKRKFH-SKHKGGKHKGGKHKG; SEQ ID NO:5), and Sp-H5 (phosphoserine-DSHAKRHHGYKRKFHEKHHSHRGY; SEQ ID NO:7) were synthesized by Shanghai Science Peptide Biological Technology Co., LTD (Shanghai, China) using the standard 9-fluorenylmethoxycarbonyl (FMOC) solid-phase method. Sequence purity (>95%) and authenticity were determined by analytical reverse-phase high-performance liquid chromatography (HPLC). The molecular mass was determined by mass spectrometry (MS). Peptides were lyophilized for storage at −70° C. and dissolved in 10 mM 4-(2-hydroxyethyl)-1-piperazineeth anesulfonic acid (HEPES) buffer at pH 7 prior to use.

### Bacterial Strains and Growth Media

Streptococcus mutans (ATCC 35668, ATCC, Manassas, USA) were cultured in Horse Blood Medium under anaerobic (85% N₂, 10% H₂, and 5% CO₂) conditions at 37° C. Cells were harvested by centrifugation (5000 rpm, 10 min), washed once with 10 mM sodium phosphate-buffered saline (PBS, pH 7.2), and resuspended in brain-heart infusion broth (BHI, Difco Laboratories, Detroit, USA) at a concentration of 10⁶ CFU/mL.

### Preparation of Tooth Slices

After approval from the Institutional Review Board of the University of Hong Kong/Hospital Authority Hong Kong West Cluster (IRB No: UW17-362), human third molars were collected from patients with written informed consent. 3×3×1.5 mm³ (number: 197) tooth slices were prepared using a water-cooled diamond saw (IsoMet low-speed saw, Buehler, Lake Bluff, IL, USA). One working surface of complete enamel tissue from each tooth slice was generated using the MicroCT Skyscan 1172 (Bruker MicroCT, Kontich, Belgium). The enamel working side of the tooth slice was polished and other sides marked with acid-proof nail polish. All tooth slices were cleaned and stored in deionized water at 4° C.

### Adsorption Capacity Assay

The adsorption of P-113, P-113-DPS, P-113-DA, and P-113-SK to HA particles (Sigma Chemical Co., St Louis, USA) was measured using the Micro BCA method with Sp-H5 as the control group. Solutions of P-113, P-113-DPS, P-113-DA, P-113-SK and Sp-H5 in 10 mM HEPES buffer (pH 7) were diluted to 10 nmol/mL concentrations, and 200 μL was placed into each tube (P-113, N=10; P-113-DPS, N=10; P-113-DA, N=10; P-113-SK, N=10; and Sp-H5, N=10). Peptide solutions were mixed with (n=5) or without (n=5) HA particles (20 mg/tube) in each group. Tubes were placed onto a rotator overnight at 37° C. for complete reaction. Tubes containing the peptide solution and HA were centrifuged (13000 rpm, 1 min) and the supernatant collected. Bovine serum albumin (BSA) standard solutions (working range=5-250 μg/mL) and working reagent were prepared using the Pierce BCA Protein Assay Kit (Rockford, IL, USA). Working reagents (100 μL/well) were added to a 96-well plate, followed by standard and peptide solutions (100 μL/well). The Plate was incubated in the dark for 30 min and the absorbance (562 nm) measured using a plate reader. The concentration (C) of the BSA standard solution was linear with the absorbance (A) at 562 nm. The function of C=a*A+b was determined (a and b as constants). The final concentration (the peptide solution mixed with HA) and the initial concentration (the peptide solution without HA) were computed using the function and the generated absorbance data of the peptide solution mixed with and without HA.

Adsorption Time Assay The adsorption time of peptides to the tooth enamel was also tested using the Micro BCA method. Solutions of P-113, P-113-DPS, P-113-DA, and P-113-SK in 10 mM HEPES buffer (pH 7) were diluted to 10 nmol/mL. Enamel working surfaces of tooth slices (N=20) were exposed to P-113 solution (n=5), P-113-DPS solution (n=5), P-113-DA solution (n=5), and P-113-SK solution (n=5), respectively. The remaining extraneous surfaces were covered with wax. Tubes were placed into a rotator at 37° C. At t=0, 5, 20, 40, 60, and 120 min, 100 μL of peptide solution was removed and added to BSA standard solutions (100 μL/well) and working reagent (100 μL/well) in a 96-well plate. After a 30-min incubation, absorbance was measured. A function of C=a*A+b was determined (a and b are constants). The concentrations of the peptide solution at different time points (C0, C1, C2, C3, C4, and C5 at 0, 5, 20, 40, 60, and 120 min, respectively) were computed based on absorbances (A0, A1, A2, A3, and A4, respectively).

### Anti-Planktonic Bacterial Ability

Two-fold serial dilutions (in 10 mM HEPES) of P-113, P-113-DPS, P-113-DA, P-113-SK, and Sp-H5 prepared in 25% BHI medium were pipetted into a 96-well cell culture plate at final concentrations from 0.125-128 μmol/mL [100 μL/well]. Each well was seeded with 10 μL bacterial suspension at a final concentration of 10⁶ CFU/mL. Sp-H5 with bacterial cells and 25% BHI medium with and without bacterial cells in 10 mM HEPES buffer (pH 7) were used as the positive, negative, and blank control group, respectively. After a 24-h incubation, absorbance was measured at 595 nm using a microplate reader to assess cell growth. The minimal inhibitory concentration (MIC) was defined as the lowest concentration of peptide resulting in at least 90% reduction in absorbance compared to the negative control group. The minimal bactericidal concentration (MBC) was defined as the lowest concentration of peptide resulting in no colony formation on agar plates after incubation. 10 μL of cell suspension from each well were plated on horse blood agar plates and incubated at 37° C. for 48 h. All determinations were conducted in triplicate experiments.

### Anti-Biofilm Activity

S. mutans biofilms were cultured on enamel working surfaces in a 96-well plate. Tooth slices (N=32) were autoclave-sterilized prior to anti-biofilm activity testing. 200 μL S. mutans in BHI (10⁶ CFU/mL) was added to each well. After a 24-h incubation at 37° C., each tooth slice was washed once with PBS. Tooth slices were incubated in a new 96-well plate at 32, 64, and 128 μmol/mL concentrations with P-113+BHI, P-113-DPS+BHI, P-113-DA+BHI, P-113-SK+BHI, Sp-H5+BHI, or BHI (control) in duplicate for 24 h at 37° C. (in 10 mM HEPES). After incubation, tooth slices were washed once with PBS. One tooth slice at random in each group was stained with the LIVE/DEAD BacLight Bacterial Viability Kit (L7012, Thermo Fisher Scientific, Waltham, USA) and incubated in the dark for 30 min. Fluorescence images were obtained using confocal laser scanning microscopy (CLSM, Fluoview 1000, Olympus, USA). A separate tooth slice was used for the counting of colony-forming units (CFU). Tooth slices were placed in 1 mL BHI solution, and adherent biofilms were removed from the enamel surfaces by sonication for 30 sec. Ten-fold serial dilutions of the suspensions were plated in duplicate on horse blood agar. CFU's were counted after 48-h incubation. Three independent biofilm experiments were performed.

### Anti-Bacterial Adhesion Ability

Tooth slices (N=32) were immersed in 8, 16, and 32, pmol/mL P-113, P-113-DPS, P-113-DA, P-113-SK, and Sp-H5 solutions (in 10 mM HEPES) or 10 mM HEPES buffer (control, 100 μL/well) in duplicate at 37° C. overnight. After overnight incubation, they were placed in a 96-well plate, followed by 200 μL S. mutans in BHI (10⁶ CFU/mL) and incubation at 37° C. for 5 h. Tooth slices were washed once with PBS, one tooth slice at random from each group was stained, and fluorescence images obtained by CLSM. Separate tooth slices were used for CFU counting. Three independent biofilm experiments were performed.

### Demineralization Assay

Tooth slices (N=30) were immersed in 8 μmol/mL P-113, P-113-DPS, P-113-DA, P-113-SK, Sp-H5 (n=5 for each) solutions (in 10 mM HEPES) or HEPES (control) overnight. Tooth slices were incubated in 2 mL demineralization solution (2.2 mM CaCl₂), 2.2 mM NaH₂PO₄, 0.05 M acetic acid, pH 4.5) for 7 days at 37° C. Lesions depths were analyzed by MicroCT Skyscan 1172. Mineral calcium (Ca) or phosphorus (P) concentrations before and after immersion were measured using Inductively Coupled Plasma Optical Emission Spectrometer (ICP-OES; Spectro Arcos, Kleve, Germany). Ca/P loss was expressed as mM/cm². The mean values of Ca/P loss were analyzed for each group using ANOVA at the 1% c significance level.

### Remineralization Assay

The enamel working surface of tooth slices (N=30) was acid-etched in 37% phosphoric acid for 1 min and rinsed with deionized water for 1 min. The slices were immersed in 8 μmol/mL P-113, P-113-DPS, P-113-DA, P-113-SK, Sp-H5 (n=5 for each) solutions (in 10 mM HEPES) or HEPES (control) overnight. All tooth slices were incubated in 7 mL remineralization solution (2.58 mM CaCl₂)·2H₂O, 1.55 mM KH₂PO₄, 10 mg/L NaF, 180 mM NaCl, 50 mM Tris-HCl, pH 7.6) for 24 h. The enamel surface and cross-section morphology were analyzed by FE-SEM (Hitachi, S4800). Ca/P concentrations in the solutions before and after immersion were calculated by ICP-OES. The mean values for Ca/P gain were analyzed using ANOVA at the 1‰ significance level.

### Cytocompatibility Test

The toxicity of peptides was tested using MC3T3-E1 cells (ATCC 2593, ATCC, Manassas, USA). MC3T3-E1 cells (5×10³) were seeded in a 96-well plate (N=72). After 24 h, cells were exposed to peptide solutions (P-113, P-113-DPS, P-113-DA, P-113-SK, or Sp-H5) for 24 h at four different concentrations: 4, 8, 16, and 32 μM mol⁻¹, in triplicate. Cells not exposed to peptides served as the control group. MC3T3-E1 cell proliferation after peptide exposure was evaluated using Cell Counting Kit-8 assay (CCK-8 assay, Sigma-Aldrich, USA).

Proliferation of MC3T3-E1 cells on peptide-coated enamel surface was tested. Tooth slices (N=48) were immersed in sterilized peptide solutions (8 μM mol⁻¹ P-113, P-113-DPS, P-113-DA, P-113-SK, or Sp-H5, n=8) or deionized water (control, n=8) for 30 min at 37° C. MC3T3-E1 cells (5×10³) were seeded onto the enamel working surfaces in a 96-well plate. After culturing for 1 and 3 days, three tooth slices in each group were used to test the proliferation of MC3T3-E1 cells by CCK-8 assay. The one tooth slice left in each group was stained by CellTracker Green (C7025, Thermo Fisher Scientific, USA). Fluorescence images were taken with CLSM.

### Molecular Dynamics Analysis of Bioactive Peptides

Three systems were constructed using the software GROMACS 5.0.4: (1) peptide adsorption onto the HA system, (2) peptide-HA complexes in the demineralization system, and (3) peptide-HA complexes in the remineralization system. Peptide structures were simulated by Swiss-PdbViewer. The initial structures of the peptide molecules were built on the (010) surface of HA crystals. The 100-ns dynamic simulation was carried out for every system. When the reaction reached equilibrium, the adsorption mechanism and occupancy situation of peptides onto HA were analyzed. The demineralization system was introduced onto the peptide-HA complexes surface (after equilibrium of peptide adsorption onto HA) based on the ion ratio [acetate ions (Ace⁻)/Ca²⁺/phosphate ions (PO₄³⁻)] in demineralization solution. Remineralization systems were built on the peptide-HA complex surface according to the ion ratio (Ca²⁺/PO₄³⁻) in the remineralization solution. Molecular dynamic reactions among ions, peptides, and HA were analyzed. Binding energy was used to analyze the reaction of peptides and ions, and HA and ions.

### Statistical Analysis

Statistical analyses were performed using SPSS 26.0 software (IBM Corporation, Armonk, NY, USA). ANOVA was used to examine means with more than two groups, and Tukey HSD tests were used to test their mutual significance.

### Results

### Adsorption Evaluation

The adsorption amount of P-113, P-113-DPS, P-113-DA, P-113-SK, and Sp-H5 with HA particles is shown in Table 1. A linear function C=316.03A−17.302 (R=0.9996) was built. Using this function, initial and final concentrations of peptides mixed with HA particles were computed. Using one-way ANOVA, a significant difference was detected in the concentration change among groups (P<0.001). By Tukey HSD multiple comparisons, P-113-DPS exhibited stronger adsorption capacity with HA particles than Sp-H5 and all other solutions.

TABLE 1
Adsorption amount for the indicated peptides with HA Particles.

| | Initial | Final | | | Multiple |
|---|---|---|---|---|---|
| | concentration | concentration | | | comparison |
| | (Mean ± SD, | (Mean ± SD, | Concentration | | (Concentration |
| Group | nM mL⁻¹) | nM mL⁻¹) | change (95% CI) | p value | change) |
| P-113 | 10.15 ± 0.35 | 9.86 ± 0.23 | 0.29 (−0.01-0.61) | <0.001 | [2] > [5] > [1] |
| | | | [1] | | [2] > [4] > [3] |
| P-113-DPS | 10.37 ± 0.36 | 6.56 ± 0.78 | 4.13 (3.28-4.88) | | [1] = [3] |
| | | | [2] | | |
| P-113-DA | 10.28 ± 0.36 | 10.05 ± 0.99 | −0.17 (−0.55-0.40) | | [4] = [5] |
| | | | [3] | | |
| P-113-SK | 10.32 ± 0.33 | 7.71 ± 0.39 | 2.61 (2.05-3.47) | | |
| | | | [4] | | |
| Sp-H5 | 10.00 ± 0.33 | 6.73 ± 0.12 | 3.27 (2.92-3.58) | | |
| | | | [5] | | |

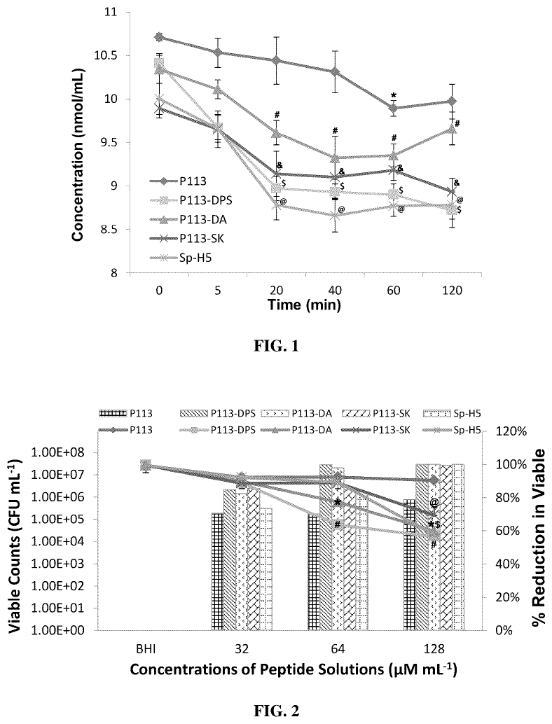

The adsorption time of P-113, P-113-DPS, P-113-DA, P-113-SK, and Sp-H5 with enamel was also investigated (FIG. 1). The concentrations of peptide solutions at different time points (0, 5, 20, 40, 60 and 120 min) were calculated based on the linear function C=317.5A−21.406 (R=0.9987) using their absorbance values. By one-way ANOVA, a significant decrease in concentration was observed from 0 to 20 min in the P-113-DPS solution (P<0.01). There was no significant difference in concentration at 40, 60, and 120 min. The majority of adsorption reactions between P-113-DPS and enamel occurred within 20 minutes. P-113-DA, P-113-SK, and Sp-H5 had similar adsorption time curves. However, P-113 showed a slower adsorption reaction with a significant decrease in concentration at 60 minutes.

### Anti-Biofouling Activity

The susceptibility assay for planktonic S. mutans in P-113, P-113-DPS, P-113-DA, P-113-SK, and Sp-H5 is summarized in Table 2. P-113-DPS showed the strongest anti-planktonic S. mutans activity based on the lowest MIC and MBC values in the group.

TABLE 2
MIC and MBC of the indicated peptides against S. mutans.

| Peptides | MIC (μM mL⁻¹) | MBC (μM mL⁻¹) |
|---|---|---|
| P-113 | 32 | >128 |
| P-113-DPS | 8 | 16 |
| P-113-DA | 16 | 32 |
| P-113-SK | 32 | 64 |
| Sp-H5 | 8 | 32 |

After 24 h of exposure of S. mutans biofilms to P-113, P-113-DPS, P-113-DA, P-113-SK, and Sp-H5 solutions with different concentrations, the majority of S. mutans were dead (data not shown) as determined by CLSM images of P-113-DPS and P-113-DA with concentrations of 64 and 128 μmol/mL. CLSM images of P-113-SK and Sp-H5 show similar results only at 128 μmol/mL (data not shown). In contrast, the majority of S. mutans in BHI and P-113 were alive. In addition, SEM imaging showed S. mutans cell membrane damage with various punctures as a result of incubation with P-113-DPS (data not shown).

Approximately 99% S. mutans were killed by 64 μmol/mL P-113-DPS or P-113-DA, and 128 μmol/mL P-113-SK or Sp-H5 using quantitative approaches. In contrast, 79% S. mutans were killed by 128 μmol/mL P-113. Viability counts reduced significantly from 64 μmol/mL P-113-DPS [5.47E+04 (CFU/mL)] or P-113-DA [6.04E+05 (CFU/mL)], and 128 μmol/mL P-113-SK [1.53E+05 (CFU/mL)] or Sp-H5 [2.0E+04 (CFU/mL)], compared with BHI [2.62E+07 (CFU/mL)](P<0.001, FIG. 2). However, only 87% S. mutans were killed by P-113 at 160 μmol/mL concentration.

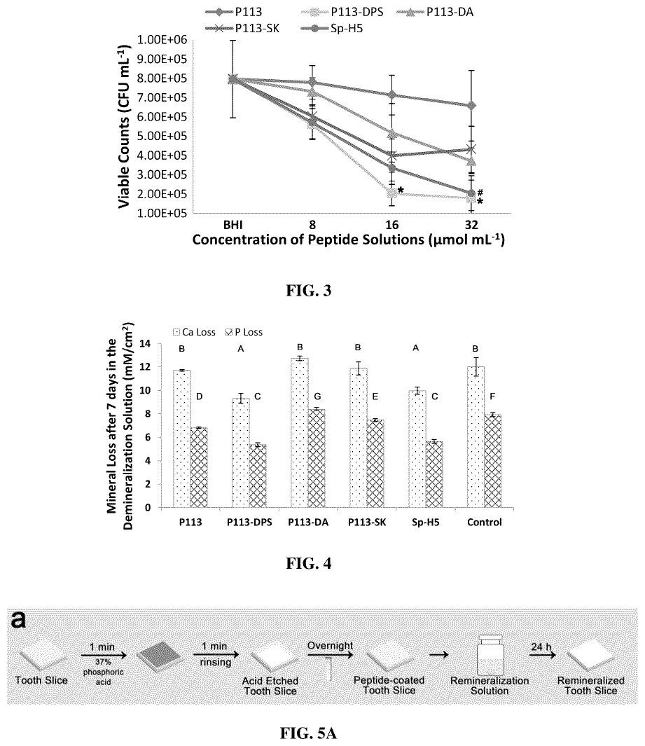

The CLSM images of the non-coated enamel surfaces revealed more live S. mutans in the control group than P-113-DPS-coated and Sp-H5-coated enamel surfaces (data not shown). Viable counts of S. mutans were significantly reduced with 16 μmol/mL P-113-DPS-coated [2.03E+05 (CFU/mL)] or 32 μmol/mL of Sp-H5-coated [2.04E+05 (CFU/mL)] enamel surfaces compared with non-coated enamel surfaces [7.96E+05 (CFU/mL)](P<0.001, FIG. 3). Significant difference was not detected using different concentrations of P-113-DPS. No significant decrease was detected in other groups. Among all peptide solutions, P-113-DPS showed the most potent inhibitory effects on S. mutans adhesion at the lowest concentration tested (16 μmol/mL).

### Demineralization and Remineralization Properties

After enamel surfaces were exposed to demineralization solutions for 7 days, the MicroCT graphs and 3D models of lesions in P-113, P-113-DPS, P-113-DA, P-113-SK, Sp-H5, and control group showed irregular morphology with various lesion areas and depths (data not shown). The maximum lesion depth in P-113, P-113-DPS, P-113-DA, P-113-SK, Sp-H5, and control group was 115 μm, 75 μm, 110 μm, 100 μm, 85 μm, and 115 μm, respectively. The cross-section of the lesion showed higher mineral density in the outermost layer than the one below in all groups. ICP-OES mineral quantification showed Ca and P loss from P-113-DPS-coated or Sp-H5-coated enamel slices decreased significantly compared with those from P-113-coated, P-113-DA-coated, P-113-SK-coated or non-coated enamel slices (P<0.001, FIG. 4). Significant differences were not detected in Ca losses between the control group and P-113, P-113-DA, or P-113-SK groups. For the selected peptide solutions, P-113-DPS and Sp-H5 demonstrated a stronger ability to inhibit demineralization.

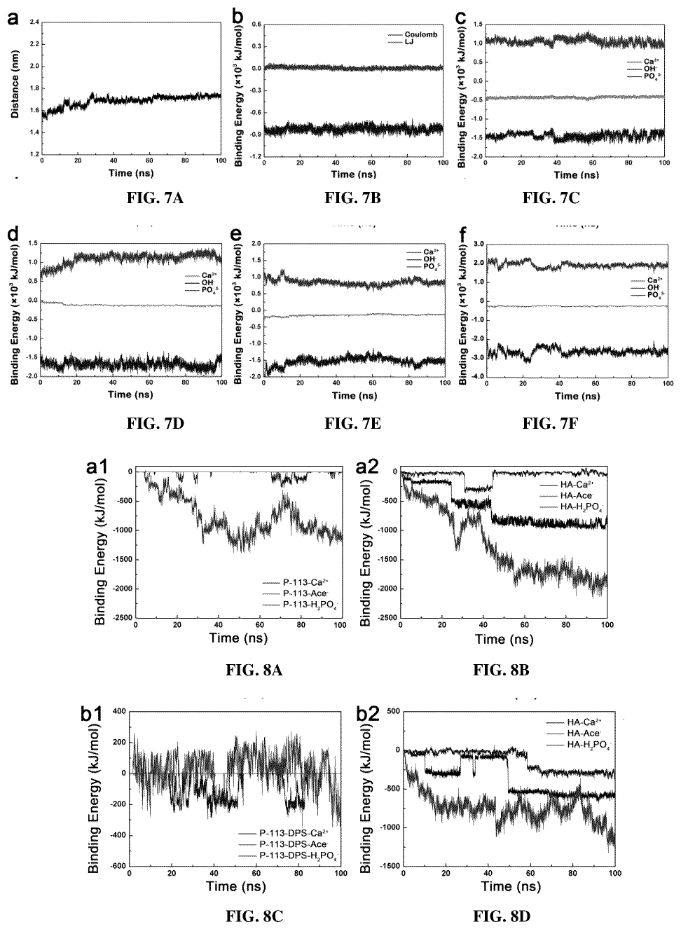

After exposure of enamel surfaces to remineralization solutions for 24 h (FIG. 5A), the generation of new HA crystals as imaged by FE-SEM were formed on demineralized enamel slices in all groups. Cross-sectional micrographs of the remineralized enamel slices clearly showed a newly regenerated layer tightly conjoined to its underlying natural enamel. The thickness of the regenerated crystal layer on the enamel surface for P-113-DPS (8.5 μm) was qualitatively thicker than P-113 (6 μm), P-113-DA (7 μm), P-113-SK (7 μm), Sp-H5 (6 μm), and controls (4 μm). The crystal macrostructure of the regenerated layer formed needle-like projections and the crystal microstructure of the regenerated layer formed hexagonal prism-like projections, both of which are morphologically similar to natural enamel. As shown in FIG. 7B, ICP-OES mineral quantification showed significant P gain in the P-113-DPS group compared with the control group (D>F, P<0.001), significant Ca gain in all treatment groups compared with the control group (B>C, P<0.001), and significant Ca gain in the P-113-DPS group compared with other groups (A>B, A>C, P<0.001).

### Cytocompatibility

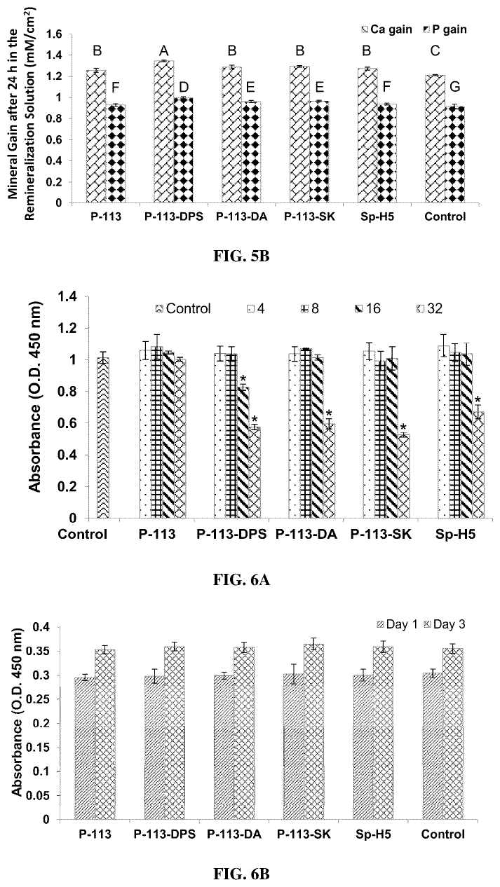

After 24 h of incubation, there was no significant absorbance difference in all concentrations of P-113; 4 and 8 μM mL⁻¹ P-113-DPS; 4, 8, and 16 μM mL⁻¹ P-113-DA; 4, 8, and 16 μM mL⁻¹ P-113-SK, compared with the control group (FIG. 6A). After 3 days incubation, MC3T3-E1 cell proliferation on 8 μM mL⁻¹ peptide-coated enamel surfaces was not significantly different among all groups (FIG. 6B). Results from fluorescent images were consistent with the quantitative CCK-8 assay. MC3T3-E1 cells were stained green and exhibited a spindle like morphology in all groups after 3 days incubation. During incubation for 3 days, 8 μM mL⁻¹ peptide-coated groups showed significantly increased numbers of live cell compared with the control group. These results indicated that at a concentration of 8 μM mL⁻¹, the peptides exhibited good cytocompatibility, and can be used for tooth surface applications.

Md Simulations—Adsorption of Peptides onto the HA System

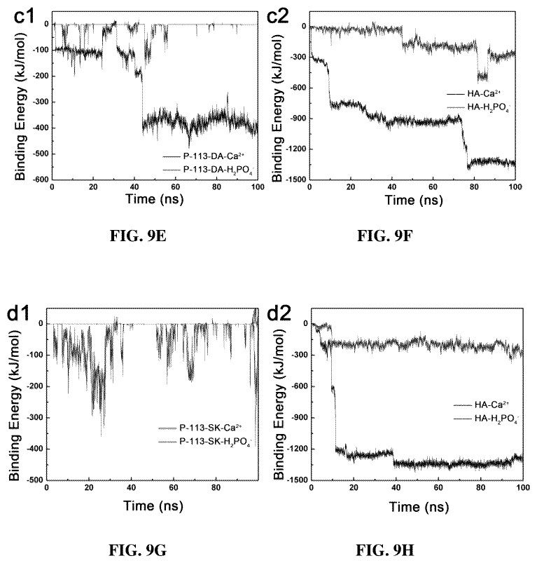

The centroid distance between P-113 and HA was within 1.6-2.4 nm without any large fluctuations evident (FIG. 7A-F), indicating a high degree of binding stability between P-113 and HA. The Lennard-Jones potential was sustained horizontally (FIG. 9B, red curve), indicating no van der Walls forces in the P-113-HA adsorption system. However, the Coulombic potential decreased at the onset and quickly reached equilibrium (<5 ns) (IG. 7B, blue curve), demonstrating the presence of electrostatic forces. The centroid distances and potential evaluations of the other peptide-HA adsorption systems were similar to the P-113-HA system. Electrostatic interaction was the main driving force in the adsorption system, and the adsorption process itself was fast (<5 ns). FIGS. 7C-7F show binding energies between the key constituents of HA and peptides (FIG. 7C, P-113; FIG. 7D, P-113-DPS; FIG. 7E, P-113-DA; and FIG. 7F, P-113-SK, respectively). The red curve initially rose, indicating the presence of repulsive forces between peptides and HA Ca²⁺ ions. The blue curve decreased at the initial simulation time (<5 ns), indicating the presence of attractive forces between peptides and HA PO₄³⁻ ions. There was a slight decrease in the gray curve, indicating few attractive forces between peptides and HA OH⁻ ions. After a comprehensive analysis of the various binding energies, negatively charged PO₄³⁻ was the main functional component in HA contributing to the adsorption of peptides.

The stable and unique molecular conformations of peptide adsorption onto HA at 100 ns scale were examined. The positive charged amino acid residues Lys2, Arg3, Lys10, and amino-terminus in P-113 adsorbed to HA. The positive charged amino acid residues Lys2, Arg3, Arg9, Lys10, and amino-terminus in P-113-DPS adsorbed with HA, while there was a large distance between DPS and HA. A similar adsorption was found in P-113-DA (Lys2, His5, Arg9, Lys10, and amino-terminus) and HA when DA was separate from HA. Apart from Lys2, Arg3, Arg9, Lys10, and the amino-terminus, more positively charged amino acid residues Lys14, Lys16, Lys19, Lys21, Lys24, and Lys26 in P-113-SK adsorbed to HA. The electrostatic interaction between positive charged lysine, arginine, and the amino-terminus of the peptides and negative charged PO₄³⁻ of the HA surface was the consistent binding pattern between peptides and HA. In comparing the binding energies of the four peptide-HA adsorption systems, the sequence of binding energies were as follows: P-113-SK (−1000 kJ/mol)<P-113-DA (−800 kJ/mol)=P-113 (−800 kJ/mol)<P-113-DPS (−700 kJ/mol).

### The Peptide-HA Complexes in the Demineralization System

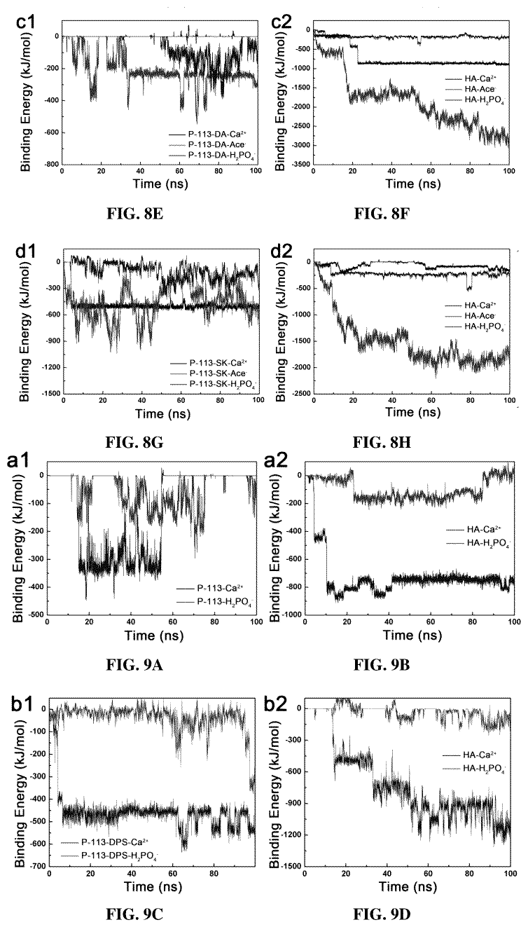

The average binding energies between ions (Ace⁻, Ca²⁺, and H₂PO₄) and the peptide-HA complexes in the demineralization system are illustrated in FIG. 8A-10H. The largest decrease in average binding energy observed was Ace and HA, implicating Ace as the most critical component to react with Ca²⁺ of the HA in the demineralization system. The sequence of the average binding energy between Ace and the peptide of the complexes was P-113-HA (−1000 kJ/mol)<P-113-SK-HA (−450 kJ/mol)<P-113-DA-HA (−200 kJ/mol)<P-113-DPS-HA (0 kJ/mol). The sequence of the average binding energy between Ace and the HA of the complexes was P-113-DA-HA (−2800 kJ/mol)<P-113-HA (−1800 kJ/mol)=P-113-SK-HA (−1800 kJ/mol)<P-113-DPS-HA (−1200 kJ/mol).

### The Peptide-HA Complexes in the Remineralization System

As shown in FIG. 9A-9H, the binding energy of Ca²⁺/H₂PO₄⁻ and HA of the peptide-HA complex fluctuates in a step-wise manner. A considerable decrease was observed in the binding energy between Ca²⁺/H₂PO₄⁻ and HA in the remineralization system. The sequence of the average binding energy between Ca²⁺ and the HA of the complexes was P-113-DA-HA (−1400 kJ/mol)<P-113-SK-HA (−1350 kJ/mol)<P-113-DPS-HA (−1050 kJ/mol)<P-113-HA (−750 kJ/mol). The sequence of the average binding energy between H₂PO₄ and the HA of the complexes was P-113-DA-HA (−300 kJ/mol)<P-113-SK-HA (−200 kJ/mol)<P-113-HA (−150 kJ/mol)<P-113-DPS-HA (−100 kJ/mol). The sequence of the average binding energy between Ca²⁺ and the peptide of the complexes was P-113-DPS-HA (−450 kJ/mol)<P-113-DA-HA (−350 kJ/mol)<P-113-HA (0 kJ/mol)=P-113-SK-HA (0 kJ/mol). There was no binding energy between H₂PO₄ and the peptides in the complexes.

In summary, a dual-bioactive peptide, P-113-DPS, was constructed with anti-biofouling and mineralizing abilities. FIG. 10 is schematic diagram illustrating a hypothesized mechanism on how P-113-DPS confers its bioactivity. In Step 1, DPS with highlighted amino acid residues Ser₁₃-p and Ser₁₄-p is shown in the outermost layer of the P-113-DPS-HA adsorption system. P-113-DPS adheres to the enamel surface through electrostatic attraction forces between positively charged amino acid residues Lys₂, Arg₃, Arg₉, Lys₁₀, and amino-terminus in P-113-DPS and negatively charged PO₄³⁻ of enamel. In Step 2, P-113-DPS is destructive against planktonic S. mutans and S. mutans biofilm by localizing at the bacterial surface, cross-linking with bacterial membrane phospholipids, and increasing the permeability of the membrane and forming perforations. In Step 3, the coating of P-113-DPS on enamel inhibits the adhesion of S. mutans through preferential occupation for binding sites in addition to its inherent antibacterial ability. In Step 4, Arg₃ is attracted to Ace while Ser13-p attracts to Ca²⁺ in compensation. Ser14-p with negative charges is in the outermost layer. Coating of P-113-DPS on enamel protects the enamel surface against the acid microenvironment via the electrostatic repulsive force of DPS in the outermost layer and space occupancy of P-113-DPS on the enamel surface. In Step 5, Ser14-p and carboxyl are attracted to Ca²⁺ by coordinate bonding and His₄ to H₂PO₄ by hydrogen bonding. Although Ser13-p does not attract to Ca²⁺ in this image, it does have the opportunity to attract Ca²⁺ during these reactions, which might occur in another time. Coating of P-113-DPS on the demineralized enamel surface attracts more Ca²⁺ from the remineralization solution and quickly induces nucleation and hexagonal prism-like crystal growth due to the presence of DPS.

The disclosed methods and compositions can be further understood in view of the following paragraphs.

1. An isolated peptide comprising an antimicrobial domain and a phosphoserine domain, wherein the antimicrobial domain comprises a histatin 5 segment, wherein the histatin 5 segment comprises a histatin 5 protein, a variant thereof, a portion thereof, or a variant of a portion thereof, wherein the phosphoserine domain comprises a phosphorylated serine,
wherein upon contact with a tooth, the isolated peptide binds to the tooth enamel, reduces or prevents tooth decay, reduces or prevents bacterial biofilm formation on the tooth surface, reduces or prevents dental plaque formation, reduces or prevents bacterial adhesion to the tooth surface, induces or increases mineralization, or combinations thereof.

2. The peptide of paragraph 1, wherein the antimicrobial domain comprises AKRHHGYKRKFH (SEQ ID NO:2) or a sequence comprising at least 90% sequence identity to AKRHHGYKRKFH (SEQ ID NO:2).

3. The peptide of claim 1 or 2, wherein the histatin 5 segment comprises the sequence DSHAKRHHGYKRKFHEKHHSHRGY (SEQ ID NO:1) or a sequence comprising at least 90% sequence identity to DSHAKRHHGYKRKFHEKHHSHRGY (SEQ ID NO:1).

4. The peptide of any one of paragraphs 1-3, wherein the phosphoserine domain comprises two or more phosphorylated serines.

5. The peptide of any one of paragraphs 1-4, wherein the phosphoserine domain is at the C-terminus of the isolated peptide.

6. The peptide of any one of paragraphs 1-5, wherein the peptide comprises the sequence AKRHHGYKRKFH-SpSp (SEQ ID NO:4), wherein each Sp independently represents a phosphorylated serine.

7. An oral care composition or device comprising the isolated peptide of any one of claims 1-6.

8. The oral care composition or device of paragraph 7 comprising a plurality of copies of the isolated peptide.

9. The oral care composition or device of paragraph 8 further comprising a pharmaceutically acceptable excipient, preferably wherein the excipient is suitable for administration to oral cavity.

10. The oral care composition or device of paragraph 8 or 9, wherein the composition or device is selected from mouthwash, toothpaste, dental cream, oral spray, a rinse, a gargle, a gum, a tablet, an oral lozenge, dental floss, a tooth whitening strip or solution, a cleaning solution, a toothpick, a toothbrush, a varnish, or composite resin.

11. A method comprising applying the oral care composition or device of any one of claims 8-10 to teeth and/or oral cavity of a subject.

12. A method of reducing, preventing, and/or repairing tooth decay in a subject in need thereof, the method comprising contacting the subject's teeth and/or oral cavity with a peptide comprising the sequence AKRHHGYKRKFH-SpSp (SEQ ID NO:4) or a sequence comprising at least 90% sequence identity to AKRHHGYKRKFH-SpSp (SEQ ID NO:4).

13. A method of reducing or preventing enamel demineralization and/or promoting enamel mineralization in a subject in need thereof, the method comprising contacting the subject's teeth and/or oral cavity with a peptide comprising the sequence AKRHHGYKRKFH-SpSp (SEQ ID NO:4) or a sequence comprising at least 90% sequence identity to AKRHHGYKRKFH-SpSp (SEQ ID NO:4).

14. The method of paragraph 12 or 13, wherein the peptide is comprised in composition or device selected from mouthwash, toothpaste, dental cream, oral spray, a rinse, a gargle, a gum, a tablet, an oral lozenge, dental floss, a tooth whitening strip or solution, a cleaning solution, a toothpick, a toothbrush, a varnish, or composite resin,
wherein the composition or device comprises a plurality of copies of the peptide.

15. The method of any one of paragraphs 12-14, wherein the peptide is in an amount effective to reduce or prevent tooth decay, reduce or prevent bacterial biofilm formation, reduce or prevent dental plaque formation, reduce or prevent bacterial adhesion to tooth surfaces, inhibit growth and/or proliferation of oral bacteria, induce or increase enamel mineralization, or combinations thereof.

16. The method of paragraph 15, wherein the bacteria is selected from Streptococcus mutans, Streptococcus faecalis, Streptococcus sobrinus, Lactobaccilus acidophilus, Lactobacillus casei, or combinations thereof.

### Example 2. Trifunctional AMPs that Exhibits Antibacterial and Remineralizing Properties

The present studies designed a previously undisclosed functional AMPs with three parts: a non-specific AMP (P-113), a specific targeting domain (C8), and an enhancing remineralization domain. Two phosphoserines (DPS) and 24 amino acids DSSDSSDSSDSSDSSDSSDSSDSS (8DSS) (SEQ ID NO:11) were selected as the candidates for enhancing remineralization [18, 19]. Hence, four multi-functional AMPs were fabricated including C8-P-113-DPS, DPS-C8-P-113, C8-P-113-8DSS, and 8DSS-C8-P-113 in this study.

The first aim was to determine the optimal multi-functional AMP that possessed the best bioactive properties among C8-P-113-DPS, DPS-C8-P-113, C8-P-113-8DSS, and 8DSS-C8-P-113. The following bioactive properties were assessed: (1) anti-bacterial activity; (2) characterization of peptides; (3) the ability to promote remineralization; and (4) the ability to inhibit demineralization in the artificial caries model. Secondly, mechanistic studies were conducted and antimicrobial activity of the optimal multi-functional AMP was investigated by transcriptomic analysis. Thirdly, the optimal multi-functional AMP's activity to improve remineralization of was explored by molecular dynamics (MD) analysis. Finally, a rat caries model was established to evaluate the optimal multi-functional AMP's efficacy against dental caries and its biocompatibility.

### Materials and Methods

### Synthesis and Purification of Peptides

All peptides listed in Table 3 were synthesized (GL Biochem (Shanghai) Ltd, Shanghai, China) using the standard 9-fluorenylmethoxycarbonyl (FMOC) solid-phase synthesis methods and were purified (>95%) by reverse-phase High-Performance Liquid Chromatography (HPLC). Peptide molecular mass was characterized by Mass Spectrometry (MS).. Peptides were lyophilized stored at −70° C. and dissolved in 4-(2-hydroxyethyl)-1-piperazineethanesulfonic acid (HEPES, 10 mM, pH=7) buffer prior to use.

TABLE 3
Amino acid sequence and molecular
weight of peptides.

| Peptide | Amino acid sequence | Molecular wt |
|---|---|---|
| P-113 | AKRHHGYKRKFH | 1564.79 |
| | SEQ ID No: 2 | |
| P-113-DPS | AKRHHGYKRKFHSpSp | 1898.91 |
| | SEQ ID NO: 4 | |
| DPS P-113 | SpSpAKRHHGYKRKFH | 1898.91 |
| | SEQ ID NO: 13 | |
| P-113-8DSS | AKRHHGYKRKFHDSSDSS | 3878.73 |
| | DSSDSSDSSDSSDSSDSS | |
| | SEQ ID NO: 14 | |
| 8DSS-P-113 | DSSDSSDSSDSSDSSDSS | 3878.73 |
| | DSSDSSAKRHHGYKRKFH | |
| | SEQ ID NO: 15 | |
| C8-P-113 | TFFRLFNRAKRHHGYKRKFH | 2647.05 |
| | SEQ ID NO: 12 | |
| C8-P-113- | TFFRLFNRAKRHHGYKRKFH | Not be |
| DPS | SpSp | synthesized |
| | SEQ ID NO: 16 | successfully |
| DPS-C8-P- | SpSpTFFRLFNRAKRHHGYK | 2981.16 |
| 113 | RKFH | |
| | SEQ ID NO: 8 | |
| C8-P-113- | TFFRLFNRAKRHHGYKRKFH | 4960.99 |
| 8DSS | DSSDSSDSSDSSDSSDSSDS | |
| | SDSS | |
| | SEQ ID NO: 9 | |
| 8DSS-C8-P- | DSSDSSDSSDSSDSSDSSDS | 4960.99 |
| 113 | SDSSTFFRLFNRAKRHHGYK | |
| | RKFH | |
| | SEQ ID NO: 10 | |

Highlighted three function domains: non-specific AMP domain in bolded and italicized letters, specific targeting domain in bold, and enhancing remineralization domain in italic, respectively.

### Preparation of Tooth Slices

Human third molars were obtained from patients who provided written informed consent, and the study was approved by the Institutional Review Board of the University of Hong Kong/Hospital Authority Hong Kong West Cluster (IRB No: UW22-657). Tooth slices measuring 3×3×1.5 mm³ (total number: 217) were prepared using a water-cooled diamond saw (IsoMet low-speed saw, Buehler, Lake Bluff, USA). Micro Computed Tomography (MicroCT Skyscan 1172 Bruker, USA) was used to identify one working surface of complete enamel tissue from each tooth slice. The enamel working side of the tooth slice was polished, and the other sides were marked with acid-proof nail polish. All tooth slices were cleaned and stored in deionized water at 4° C.

### Anti Bacterial Ability of Peptides

### Anti-Planktonic Bacterial Ability

S. mutans (ATCC 35668, ATCC, Manassas, USA), S. gordonii (ATCC 35105, ATCC, Manassas, USA), and S. sanguinis (ATCC 10556, ATCC, Manassas, USA) strains were cultured in brain-heart infusion broth (BHI, Difco Laboratories, Detroit, USA) at 37° C. under anaerobic conditions (85% N₂, 10% H₂, and 5% CO₂). Bacteria were harvested by centrifugation (5000 rpm, 10 min), washed once with sodium phosphate-buffered saline (PBS, 10 mM, pH=7.2), and resuspended in BHI at a concentration of 1×10⁶ CFU mL⁻¹.

A series of two-fold dilutions of P-113, P-113-DPS, DPS-P-113, P-113-8DSS, 8DSS-P-113, C8-P-113, DPS-C8-P-113, C8-P-113-8DSS, and 8DSS-C8-P-113 were prepared in 25% BHI medium. Then they were pipetted into a 96-well cell culture plate with the final concentrations from 0.015-64 μM mL^{-1 [100} μL well⁻¹]. Each well was seeded with 10 μL S. mutans suspension, S. gordonii suspension, and S. sanguinis suspension, respectively. The negative and blank control groups were prepared using 25% BHI medium with and without bacteria, respectively, in 10 mM HEPES buffer (pH=7). The bacterial growth was assessed by measuring the absorbance at 595 nm using a microplate reader after a 24-hour incubation. Subsequently, 10 μL of bacterial suspension from each well were plated on horse blood agar plates and incubated at 37° C. for 48 h. The antibacterial activities of AMPs were evaluated. The minimal inhibitory concentration (MIC) was defined as the concentration of peptide that results in a minimum of 90% reduction in absorbance compared to the negative control group [20]. The minimal bactericidal concentration (MBC) was defined as the lowest concentration of peptide that results in no colony formation on agar plates after incubation. AMPs with weaker antibacterial activity (higher MIC) were excluded in the subsequent experiments. A 24 h growth curve of S. mutans in the ½×MBC of AMPs with stronger antibacterial activity was investigated.

Anti-S. mutans Adhesion Ability

Tooth slices (N=40) were immersed in 2×MBC, 4×MBC, or 8×MBC of AMPs (in 10 mM HEPES, n=4 for each) with stronger antibacterial activity, or HEPES buffer (control, 10 mM, 100 μL well⁻¹, n=4) in duplicate at 37° C. overnight. After overnight incubation, the samples were transferred to a 96-well plate. Then, 200 μL of S. mutans in BHI (1×10⁶ CFU mL⁻¹) was added, followed by incubation at 37° C. for 5 hours. The tooth slices were washed once with PBS. One randomly selected tooth slice from each group was stained with the LIVE/DEAD BacLight Bacterial Viability Kit (L7012, Thermo Fisher Scientific, Waltham, USA) and incubated in the dark for 30 minutes. Confocal Laser Scanning Microscopy (CLSM, Fluoview 1000, Olympus, USA) was used to acquire fluorescence images. The left three tooth slices were used for colony-forming unit (CFU) counting. Three independent biofilm experiments were conducted.

Anti-S. mutans Biofilm Activity

S. mutans biofilms were cultivated on enamel working surfaces in a 96-well plate. The tooth slices (N=52) were autoclave-sterilized before anti-biofilm activity testing. 200 μL of S. mutans in BHI (1×10⁶ CFU mL⁻¹) was added to each well, followed by incubation at 37° C. for 24 hours. After incubation, each tooth slice was washed once with PBS. The tooth slices were then incubated in a new 96-well plate containing 2×MBC, 4×MBC, 8×MBC, 16×MBC AMPs (with stronger antibacterial activity, n=4 for each)+BHI, or BHI (control, n=4) in duplicate for 24 hours at 37° C. (in 10 mM HEPES). After incubation, tooth slices were washed once with PBS. One randomly selected tooth slice from each group was stained, and fluorescence images were obtained using CLSM. The remaining three tooth slices were used for CFU counting. Three independent biofilm experiments were conducted.

### Characterization of Peptides

### General Properties and Transmission Electron Microscopy

The general properties of peptides, including C8-P-113, DPS-C8-P-113, and 8DSS-C8-P-113, were investigated. The peptides were dissolved in a 10 mM HEPES solution at varied concentrations, with the pH adjusted to 7 using 0.1 M NaOH. The characteristics of the peptides were then observed.

Peptides (C8-P-113, DPS-C8-P-113, or 8DSS-C8-P-113) were characterized by Transmission Electron Microscopy (TEM). A suspension of 1 μM mL⁻¹ peptides were ultrasonically diluted with deionized water. The diluted suspension was smeared onto a carbon-coated copper grid (200 mesh). The grids were stained with uranyl acetate solution (2% w/v in water) for 40 s and washed twice with water. Then they were examined under TEM (Philips CM 100, Philips/FEI Corporation, USA).

### Field Emission Scanning Electron Microscope

To observe the status of the hydrogel, a concentration of 16 μM mL-1 of the peptide 8DSS-C8-P-113 was applied onto a cover slip. The hydrogel 8DSS-C8-P-113 was fixed using 2.5% glutaraldehyde (Sigma-Aldrich, USA) in PBS (10 mM, pH=7.2) overnight at 4° C. Subsequently, the hydrogel was dehydrated using a series of ethanal solutions with increasing concentrations. The dehydrated hydrogel was then subjected to the critical point drying technique. After preparation, the hydrogel sample was cut and the cross-section morphology was analysed using a Field Emission Scanning Electron Microscope (FE-SEM S4800, Hitachi, Japan).

### Stability of Peptides

Human saliva was collected from a volunteer in the morning prior to oral cleaning, which was approved by the Institutional Review Board of the University of Hong Kong/Hospital Authority Hong Kong West Cluster (IRB No: UW22-657). The collected saliva samples were centrifuged at 12,000 rpm for 20 min²¹]. The supernatant was filtered to remove any debris. Peptide solution (C8-P-113, DPS-C8-P-113, or 8DSS-C8-P-113) was added into the human saliva with the final concentration 1 mg mL⁻¹, and then was incubated at 37° C. At 0, 0.5, 1, 2, 4, and 6 h, 40 uL solutions were taken from each sample and were analysed by High Performance Liquid Chromatography (HPLC-UV, 1525 Binary HPLC Pump, 2489 UV/Visible Detector, Waters, USA).

20 μL of 16 μM mL⁻¹ solution of 8DSS-C8-P-113 was applied to the tooth surface (N=5), and self-formed a layer of hydrogel sticking to the bottom of a hand-made instrument (FIG. 2J). To simulate the average human salivary flow rate, a stable flow of deionized water at a rate of 30 mL h-1 was provided using a Syringe Pump (LSP02-1B Double Channel inject/extract, China). The drained liquid, approximately 15 mL, was collected every 30 min. A sample of 100 μL of 16 μM mL⁻¹ solution of 8DSS-C8-P-113 was added to 15 mL of deionized water to serve as the 0 min sample. After 2 h, the drained liquids collected at 30 min, 60 min, 90 min, and 120 min were tested for their concentrations using Pierce BCA Protein Assay Kit (Rockford, IL).

### Cytocompatibility Test

MC3T3-E1 cells (ATCC 2593, ATCC, Manassas, USA) were used to test the toxicity of peptides. MC3T3-E1 cells (5×10³) were seeded in a 96-well plate and cultured in the alpha minimum essential medium (α-MEM, Thermo Fisher Scientific, USA) at 37° C. under the condition (95% air, 5% CO₂). After 24 h, cells were exposed to peptide solution (C8-P113, DPS-C8-P113, or 8DSS-C8-P113 dissolved in α-MEM) for 24 h at three different concentrations: ½×MBC, MBC, and 2×MBC in triplicate. Cells exposed to α-MEM without peptide was the control group. Cells viability after peptide exposure was evaluated by cell survival rate using Cell Counting Kit-8 assay (CCK-8 assay, Sigma-Aldrich, St. Louis, USA).

Cell proliferation was tested on peptide-coated enamel surface. Tooth slices (N=40) were immersed in sterilized peptide solution (C8-P113, DPS-C8-P113, or 8DSS-C8-P113 at MBC concentration, or 16 μM mL-1 of 8DSS-C8-P113, n=8 for each) or deionized water (control, n=8) for 30 min at 37° C. MC3T3-E1 cells (5×10³) were seeded onto the peptide-coated enamel working surfaces. After culturing for 1 and 3 days, three tooth slices in each group were used to test the proliferation of MC3T3-E1 cells by CCK-8 assay. The one tooth slice left in each group was stained by CellTracker Green (C7025, Thermo Fisher Scientific). Fluorescence images were taken with CLSM.

### Remineralization Assay of Peptides

The enamel surfaces of tooth slices (N=30) were subjected to acid-etching in 37% phosphoric acid for 1 minute, followed by rinsing with deionized water for 1 minute. Solutions of C8-P113, DPS-C8-P113, 8DSS-C8-P113 (at MBC concentration, in 10 mM HEPES), or HEPES (control) were then added in 5 μL droplets onto the tooth slices (n=5 for each). To investigate the impact of hydrogel status on remineralization, 16 μM mL^{-1 8}DSS-C8-P113 (n=5) was also added in 5 μL droplets onto the tooth slices. The control group (n=5) consisted of dissolving 0.5 g of agarose powder in 100 mL of deionized water, adjusting the pH to 6.5 using 0.1 M NaOH, heating to 100° C., and keeping at 60° C. after complete dissolution before use [22]. Following natural drying of the liquids or formation of hydrogels, all tooth slices were incubated in 6 mL of remineralization solution (2.58 mM CaCl₂·2H₂O, 1.55 mM KH₂PO₄, 1 mg L⁻¹ NaF, 180 mM NaCl, 50 mM Tris-HCl, pH=7.6) for 1 day, 3 days, and 7 days, respectively [23]. The remineralization solutions were replaced daily. The enamel surface and cross-section morphology were analysed using a Field Emission Scanning Electron Microscope (FE-SEM S4800, Hitachi, Japan). The Vicker's hardness of the enamel on the tooth slices was measured using a Microhardness tester (MCT³, Anton Paar, Australia). The enamel surface was subjected to a load of 0.5 N for 10 seconds at each test point for microhardness assessment.

### Demineralization Assay in the Artificial Caries Model

Tooth slices (N=30) were autoclave-sterilized and then were immersed in C8-P113, DPS-C8-P113, 8DSS-C8-P113 solutions (at MBC concentration, in 10 mM HEPES) or HEPES (control) (n=5 for each) overnight. To investigate the impact of hydrogel status on demineralization, 16 μM mL^{-1 8}DSS-C8-P113 (n=5) was also added in 5 μL droplets onto the tooth slices. The control group (n=5) consisted of dissolving 0.5 g of agarose powder in 100 mL of deionized water, adjusting the pH to 6.5 using 0.1 M NaOH, heating to 100° C., and keeping at 60° C. after complete dissolution before use [22]. After natural drying of the liquids or formation of hydrogels, all tooth slices were put into 24-well plate. A 700 μL suspension of S. mutans (1×10⁶ CFU mL⁻¹) in 25% BHI medium with 1% sucrose was added to a 24-well plate and incubated at 37° C. for 7 days. On the 1st day, 3rd day, 5th day, and 7th day, tooth slices were transferred to new wells adding 25% BHI with 1% sucrose. After 7 days, tooth slices were scanned by MicroCT and the lesion depth and bone mineral density of lesions were evaluated by CT Analyser software (ver. 1.20.3.0+, Bruker microCT). Random five areas on each tooth slice were measured.

### Mechanism of Antibacterial Activity of the Optimal Multi-Functional Peptide by Transcriptome Analysis

### Lactate Dehydrogenase Assay

A series of two-fold dilutions of 8DSS-C8-P-113 peptide at sub-MBC levels (½×MBC, ¼×MBC, and ⅛×MBC, n=5 for each) were prepared in BHIS medium. S. mutans cells were individually exposed to the 8DSS-C8-P113 peptide solutions and cultivated for a duration of 24 h. The control group consisted of S. mutans cells cultivated in BHIS media without 8DSS-C8-P-113 (n=5). To assess the activity of lactate dehydrogenase (LDH) assay, the LDH Activity Assay Kit (MAK066, Sigma-Aldrich, USA) was used. After incubation, the samples were homogenized on ice in LDH assay buffer. The resulting homogenate was then centrifuged (3000 rpm, 15 min, 4° C.). The supernatant containing the released LDH enzyme was collected for further analysis. To quantify the LDH enzymatic activity, the absorbance at 450 nm was measured, following the instructions provided with the LDH Activity Assay Kit. The recorded absorbance values were used to calculate the percentage of ΔA450 (change in absorbance at 450 nm) relative to the untreated control. Three independent LDH assays were conducted.

### Water-Insoluble Extracellular Polysaccharides Measurement

Biofilms of S. mutans were generated in 96-well plates using different sub-MBC levels (½×MBC, ¼×MBC, and ⅛×MBC, n=5 for each) of 8DSS-C8-P113 peptide in BHIS medium. BHIS medium was the control group (n=5). To determine the water-insoluble extracellular polysaccharides (EPS) in the biofilms, the anthrone method was employed [25]. To collect the biofilms, they were subjected to sonication in PBS buffer. The resulting precipitate was obtained through centrifugation (6000 rpm, 10 min) and then resuspended in 200 μL of 0.4 M NaOH. The suspension was incubated at 37° C. for 2 h. Following another round of centrifugation (6000 rpm, 10 min), 100 μL of the supernatant was mixed with 300 μL of anthrone reagent and heated at 95° C. for 8 min. The absorbance at 625 nm was measured to quantify the EPS content. This process was repeated three times independently.

For EPS staining, biofilms of S. mutans were developed on tooth slices (N=20) in a 96-well plate using different sub-MBC levels (½×MBC, ¼×MBC, and ⅛×MBC, n=5 for each) of 8DSS-C8-P113 in BHIS medium. BHIS medium was the control group (n=5). At the start of biofilm formation, Alexa Fluor®647 (D22914; Molecular Probes™) was added, while SYTO® 9 (S34854; Molecular Probes™, Invitrogen, Carlsbad, CA) was used to stain the bacteria after biofilm formation. CLSM was utilized to image the biofilms. Each biofilm was scanned at five randomly selected positions. The three-dimensional (3D) reconstructions of the biofilms were performed using ZEN 3.3 software (Carl Zeiss, Germany). The calculation of bacteria/EPS biomass was conducted using COMSTAT²⁶

### Transcriptome Analysis

S. mutans were treated with 1 μM mL⁻¹ (½×MBC) 8DSS-C8-P113 peptide in BHI medium (pH=7) for 24 h at 37° C., and BHI medium without 8DSS-C8-P113 was used as control group. Then the cells in two groups were collected after centrifugation at 10000 rpm under 4° C. for 2 min. Total bacterial RNA was extracted by utilizing the Trizol Reagent (Invitrogen, Carlsbad, CA, USA). The quality and integrity of extracted RNA were determined by NanoDrop 2000 spectrophotometer (Thermo Scientific, Wilmington, USA) and Bioanalyzer 2100 system (Agilent Technologies, Santa Clara, CA, USA), respectively. After removing rRNA and constructing cDNA libraries, the libraries were sequenced based on NovaSeq 6000 platform (Illumina, San Diego, USA) by Shanghai Personal Biotechnology Cp. Ltd (Shanghai, China).

After quality assessment and filtering of raw data, the libraries were used for subsequent bioinformatics analysis. According to reference genome (NCBI genome sequencing GCF_009738105.1), the filtered data were annotated using Bowtie 2 software (version 2.2.6.). The value of fragments per kilobase per million mapped reads (FPKM) was used to represent the expression level of each gene. Then, DESeq R package (version 1.30.0) was used to perform differential expression analysis to identify differentially expressed genes (DEGs). The mRNAs with |log₂fold change|>1 and P<0.05 were considered as DEGs between control and experimental groups. The volcano plot and heatmap of DEGs were generated using ggplot2 R package. Gene Ontology (GO) enrichment analysis was conducted by ClusterProfiler (Bioconductor, Boston, USA) (version 3.4.4) and topGO (Elk Grove Village, USA) to determine underlying biological function, while Kyoto Encyclopedia of Genes and Genomes (KEGG, Kyoto, Japan) pathway enrichment analysis was carried out by ClusterProfiler (version 3.4.4) to investigate the potential mechanisms involved in the antimicrobial effects of 8DSS-C8-P113. The GO terms and KEGG pathways with P<0.05 were considered significantly enriched by DEGs. The ggplot2 package was applied to generate bar charts of GO terms and KEGG pathways.

### Molecular Dynamic Analysis

### The Formation of 8DSS-C8-P113 Hydrogel

Based on the experiment of 8DSS-C8-P113 forming a hydrogel structure in deionized water, MD analysis was used to stimulate the process of forming the hydrogel and create a 3D model. AlphaFold software was used to create a 8DSS-C8-P113 molecule structure. Then a simulated box measuring 10 nm×10 nm×10 nm was constructed, and 20 8DSS-C8-P113 molecules were added into the box. MD simulation was carried out using Gromacs 2019.6 program under constant temperature and pressure with periodic boundary conditions²⁷. The cluster module of Gromacs was used to analyze the simulation trajectories from 60-100 ns, with a root mean square deviation (RMSD) cut-off value of 1.2 nm. The final configuration was established.

### Remineralization System

MD simulation was employed to investigate the interaction between hydrogel structures of 8DSS-C8-P113 and HAP crystal surfaces, as well as the interactions between HAP crystal surfaces and ions (Ca²⁺, HPO₄²⁻, H₂PO₄⁻). Due to the lower hydration energy of calcium ions on the surface of HAP, HAP (100) and HAP (001) surfaces with high calcium ion content were constructed separately. The crystal model of HAP (100) has dimensions of 11.31 nm×13.07 nm×22.00 nm in the xyz direction, while the crystal model of HAP (001) has dimensions of 11.31 nm×13.06 nm×22.00 nm in the xyz direction. 20 8DSS-C8-P113 hydrogel structures, Ca²⁺, HPO₄²⁻, and H₂PO₄⁻, were added to each of the two systems (HAP (100) surface and HAP (001) surface. MD simulation was conducted using the Gromacs 2019.6 program with constant temperature and pressure and periodic boundary conditions²⁷. The two systems were simulated for 100 ns, with conformations saved every 20 ps. The interactions between peptide clusters and HAP surfaces, spatial conformations, and the distance between the centroids of the two systems were analysed at the end of each simulation. The radial distribution function of free Ca²⁺ ions and phosphorus atoms on the HAP surface, as well as the interaction energy between free phosphate ions (HPO₄²⁻, H₂PO₄) and Ca²⁺ ions on the HAP surface were also analysed. The simulation results were visualized using the built-in program in Gromacs and Visual Molecular Dynamics (VMD).

### Rat Caries Model

The rat caries model was conducted under the protocol approved by the Ethics Committee of Anhui Medical University, China (ref. no. LLSC20232002) in accordance with national and international guidelines to minimise animal suffering. Ten male specific pathogen-free Sprague-Dawley rats aged 6 weeks were selected. The rats were provided with appropriate antibiotics (ampicillin, chloramphenicol, and carbencillin 1.0 g/kg/day) in the drinking water for 3 days. Then the rats were orally infected with 0.3 mL fresh BHI medium containing S. mutans (10⁹ CFU mL⁻¹) once a day for 10 days. The infection was confirmed by oral swabbing and culturing on mitis salivarius-bacitracin (MSB) agar plates. The infected animals were randomly assigned into two groups (N=10): 16 μM mL⁻¹ of 8DSS-C8-P-113 in 10 mM HEPES (pH=7, n=5) and 10 mM HEPES (n=5, control group). Animals in each group were treated with 150 μL of the respective treatment topically using dental bonding brush for 2 min once daily for 4 weeks. The rats were fed with cariogenic Keyes 2000 diet (Jiangsu Xietong Pharmaceutical Bio-engineering Co., Ltd, Nanjing, China) and 5% sucrose water ad libitum during the inoculation of S. mutans and the 4-week treatment [28]. After the rats were sacrificed, the lower jaw was aseptically dissected and sonicated in 5.0 mL of sterile saline solution (0.9%, w/v). The obtained suspensions were streaked on MSB agar plates to estimate the population of S. mutans. The lower left molars of the rats were scanned using MicroCT to analyze the volume of dental caries. The 3D reconstructions of the tooth crown and tooth half-crown were conducted by the CTVOL software (ver. 2.3.2.0, Bruker MicroCT, USA). All molars were cut from mesial to distal, the cross sections were photographed using the stereo microscope (Nikon SM218, USA). The severity of caries was evaluated by the Keyes' scoring system which categorizes caries as enamel only (E), dentin exposed ¼ to ¾ (Ds), or more than ¾ dentin affected (Dm) [29]. The rats' weights were recorded to monitor for signs of toxicity. Additionally, various tissues from gingiva, palate, heart, liver, spleen, lung, and kidney were collected, stained with hematoxylin and eosin (H&E), and histopathologically analysed.

### Statistics

Statistical analyses were performed using SPSS 27.0 software (IBM Corporation, Armonk, NY, USA). ANOVA was utilized to assess means with more than two groups, and Tukey HSD tests were employed to determine their mutual significance. Data are presented as mean±standard deviation, and p<0.05 was regarded as statistically significant (*p<0.05, **p<0.01, ***p<0.001).

### Results

### The Optimal Peptide

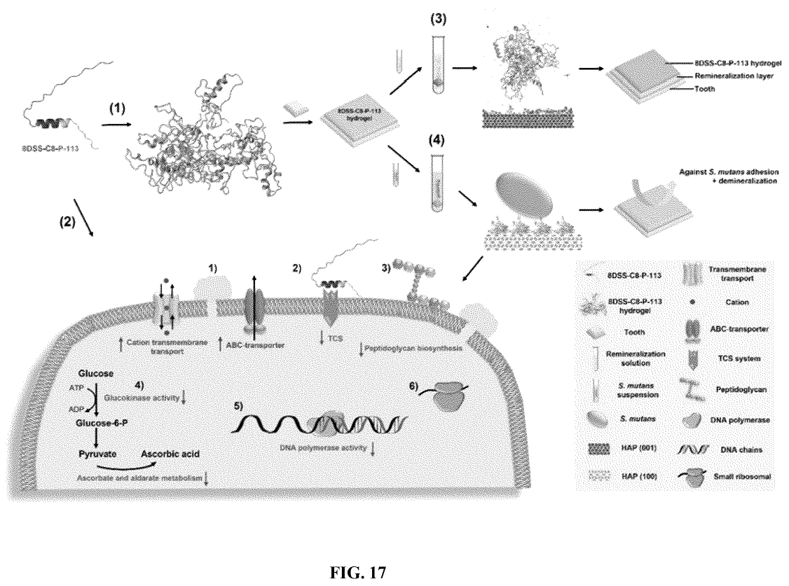

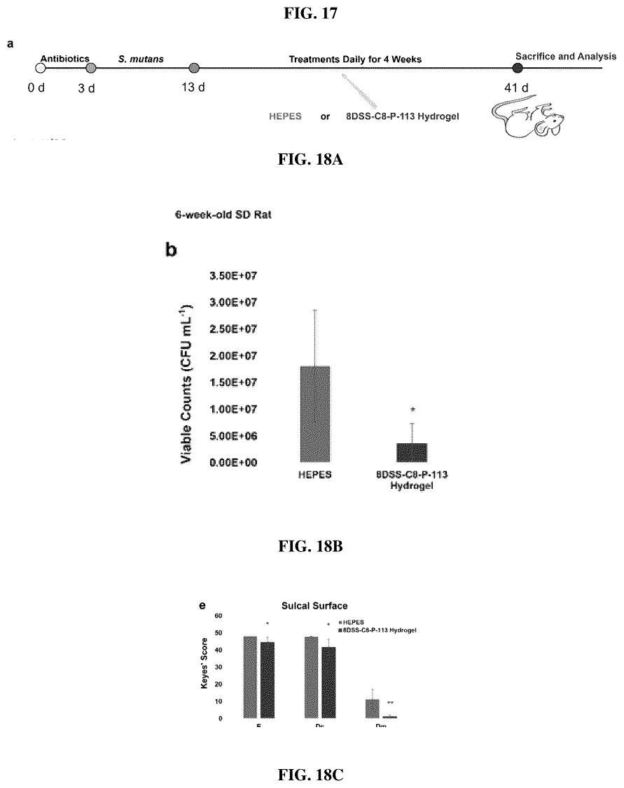

8DSS-C8-P-113 demonstrated superior performance, as summarized in Table 4. The hypothesized schematic diagram in FIG. 17 illustrates the bioactivities of 8DSS-C8-P-113. Firstly, numerous of 8DSS-C8-P-113 molecules form a hydrogel state, adhering to tooth surface. Secondly, 8DSS-C8-P-113 kills planktonic S. mutans, inhibits S. mutans' adhesion, eradicates S. mutans' biofilm, and reduces S. mutans' virulence. The probable mechanisms of 8DSS-C8-P-113 interfering S. mutans are listed. 1) 8DSS-C8-P-113 induces cell membrane perforation by up-regulating cation transmembrane transport and ABC-transporter. 2) C8 in 8DSS-C8-P-113 activates a transmembrane receptor, leading to interference with QS and inhibition of biofilm formation by down-regulation of TCS. 3) 8DSS-C8-P-113 interferes with peptidoglycan biosynthesis, resulting in osmotic lysis. 4) 8DSS-C8-P-113 interferes with glycolysis, leading to decreased virulence by down-regulating glucokinase activity, as well as ascorbate and aldarate metabolism. 5) 8DSS-C8-P-113 inhibits DNA polymerase activity. 6) 8DSS-C8-P-113 inhibits protein modification. Thirdly, when exposed to the remineralization solution for a period of 7 days, 8DSS-C8-P-113 hydrogel on the enamel surface induces the adherence of essential ions, including Ca²⁺, H₂PO₄⁻, and HPO₄²⁻, to HAP (001). This process facilitates the formation of a needle-shaped remineralization crystal layer between the 8DSS-C8-P-113 hydrogel and the tooth surface. Fourthly, 8DSS-C8-P-113 hydrogel adsorbs onto the HAP (100) surface, effectively inhibiting the adhesion of S. mutans and subsequently reducing tooth demineralization.

TABLE 4
Comparison of properties of peptides.

| | Anti-biofouling | | | | | | | | Inhibiting |
|---|---|---|---|---|---|---|---|---|---|
| | μM mL⁻¹ | | | | | | | | deminerali- |
| | MBC | | | | | | | Promoting | zation in the |
| | S. | S. | S. | Anti- | Anti- | | Cytocompat- | reminer- | artificial |
| | mutans | gordonii | sanguinis | adhesion | biofilm | Stability | ibility | alization | caries model |
| C8-P-113 | 0.03125 | 0.25 | 0.25 | 0.25 | 0.12 | − | ++ | − | + |
| | | | | (8 × | (4 × | | | | |
| | | | | MBC) | MBC) | | | | |
| DPS- | 0.25 | 4 | 2 | 1 | 0.5 | ++ | + | − | ++ |
| C8-P-113 | | | | (4 × | (2 × | | | | |
| | | | | MBC) | MBC) | | | | |
| 8DSS- | 2 | 32 | 32 | 8 | 16 | +++ | ++ | MBC: ++ | MBC: +++ |
| C8-P-113 | | | | (4 × | (8 × | | | 16 μM | 16 μM |
| | | | | MBC) | MBC) | | | mL⁻¹: +++ | mL⁻¹: +++ |

More + represents more effective and − represents ineffective.

The specific targeting agent 8DSS-C8-P-113, at a concentration of 2 μM mL⁻¹, can be used as a mouthwash to effectively eliminate planktonic S. mutans without disrupting the oral normal flora, such as S. gordonii and S. sanguinis. 8DSS-C8-P-113 with the concentration of 8 μM mL⁻¹ can be used in the form of varnish to prevent S. mutans' adhesion. Furthermore, 16 μM mL⁻¹ of it can be employed in the form of hydrogel to eradicate S. mutans' biofilm, safeguard the tooth against acid injury, and facilitate the repair of demineralized hard tissue.

### Results

Anti-Bacterial Ability Anti-Planktonic S. mutans, S. Gordonii, and S. sanguinis

The susceptibility assay for planktonic S. mutans, S. gordonii, S. sanguinis in peptides is summarized in Table 5. MICs cannot be tested because of precipitation of peptides. C8-P-113, DPS-C8-P-113, and 8DSS-C8-P-113 showed stronger anti-planktonic S. mutans activity than other peptides in the group, based on the lower MBC values (0.03 μM mL⁻¹, 0.25 μM mL⁻¹, and 2 μM mL⁻¹, respectively). Furthermore, there was an 8-fold difference in susceptibility of S. mutans (0.03 μM mL⁻¹) and other two bacteria, S. gordonii and S. sanguinis (0.25 μM mL⁻¹), to C8-P-113. S. mutans also showed susceptibility to DPS-C8-P-113 (0.25 μM mL⁻¹) and 8DSS-C8-P-113 (2 μM mL⁻¹). Only C8-P-113, DPS-C8-P-113 and 8DSS-C8-P-113, showed enhanced antimicrobial activity and specificity against planktonic S. mutans cells. Therefore, other peptides would be excluded in the following experiments.

TABLE 5
MBCs of Peptides Against Bacteria.

| | MBC (μM mL⁻¹) | | | | | | | | |
|---|---|---|---|---|---|---|---|---|---|
| | | | | | | | DPS-C8- | C8-P- | 8DSS-C8- |
| Strains | P-113 | P-113-DPS | DPS-P-113 | P-113-8DSS | 8DSS-P-113 | C8-P-113 | P-113 | 113-8DSS | P-113 |
| S. mutans | >128 | 16 | 64 | >64 | >64 | 0.03125 | 0.25 | 64 | 2 |
| S. gordonii | >128 | >64 | >64 | >64 | 64 | 0.25 | 4 | >64 | 32 |
| S. sanguinis | >128 | >64 | >64 | >64 | 64 | 0.25 | 2 | >64 | 32 |

P-113 and the modified P-113 peptides presented the broad-spectrum antibacterial activities to S. mutans, S. gordonii, and S. sanguinis. Peptides with the competence stimulating peptide fragment C8 presented specific targeting ability in this study, killing S. mutans with lower concentrations and protecting the oral normal flora, such as S. gordonii, and S. sanguinis. Based on results from C8-P-113, DPS-C8-P-113, and 8DSS-C8-P-113, it is noted that P-113 in the C-terminus exhibits better antibacterial activities to S. mutans. C8-P-113-8DSS needs a higher concentration against S. mutans, S. gordonii, and S. sanguinis. The competence stimulating peptide in the N-terminus and non-specific AMP in the C-terminus have better efficacy

### Growth Inhibition

S. mutans in the ½×MBC of C8-P-113, DPS-C8-P-113, or 8DSS-C8-P-113 solutions continued to grow approximately at the same rate within the first 5 h, compared with the control group. The absorbance of 8DSS-C8-P-113 suspension was higher due to its translucency. S. mutans started to grow exponentially from 5 hours and reached a growth plateau around 12 hours in all groups. A decrease in S. mutans growth was observed after a longer exposure to ½×MBC peptides. C8-P-113, DPS-C8-P-113, or 8DSS-C8-P-113 exhibited inhibitory effect on the S. mutans growth.

Anti-S. mutans Adhesion Ability

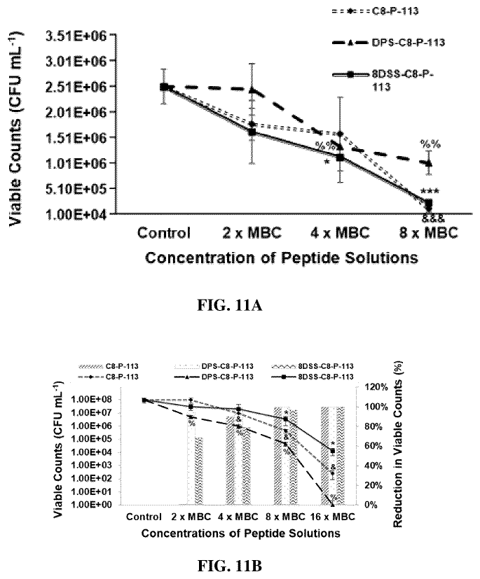

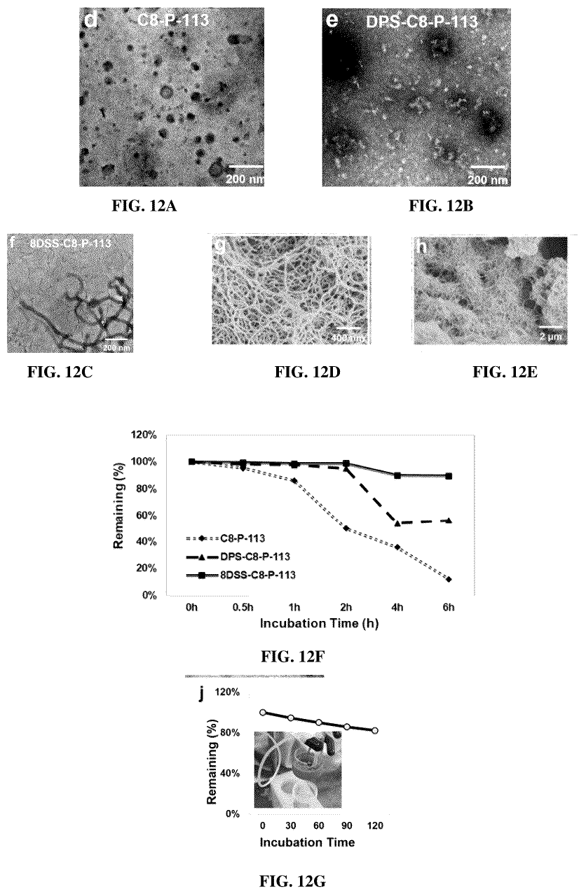

After 5 h of exposure of S. mutans to the peptide-coated enamel surfaces, the CLSM images of 8×MBC C8-P-113-coated, DPS-C8-P-113-coated, or 8DSS-C8-P-113-coated enamel surfaces revealed less live S. mutans (green) than the non-coated enamel surfaces in the control group (data not shown). The fragments of 8DSS-C8-P-113 hydrogel that formed crystal-like structures on the surface of enamel surface were stained green (indicated by arrows) and have a completely different morphology from S. mutans colonies. Some dead S. mutans (red) can be found on the enamel surface in the 8×MBC DPS-C8-P-113 group and 8×MBC 8DSS-C8-P-113 group (data not shown). The viable counts of S. mutans were significantly reduced with 8×MBC C8-P-113-coated [1.31E+05 (CFU mL⁻¹), p<0.001], 8×MBC DPS-C8-P-113-coated [1.02E+06 (CFU mL⁻¹), p<0.01], 8×MBC 8DSS-C8-P-113-coated [2.49E+05 (CFU mL⁻¹), p<0.001] enamel surfaces compared with non-coated enamel surfaces [2.52E+06 (CFU mL⁻¹)](FIG. 11A). At the concentration of 4×MBC, viable counts of S. mutans on the C8-P-113-coated enamel surface were not significantly different with that in the control group. In comparison with C8-P-113 group, DPS-C8-P-113 and 8DSS-C8-P-113 showed more potent inhibitory effects on the S. mutans adhesion.

Anti-S. mutans Biofilm Ability

After 24 hours of exposure of S. mutans biofilms to C8-P-113 and DPS-C8-P-113 solutions, the majority of S. mutans were dead (data not shown) as shown in CLSM images with concentrations of 8×MBC and 16×MBC. In contrast, the majority of S. mutans in the control group were alive (data not shown). In the 8×MBC and 16×MBC concentrations of the 8DSS-C8-P-113 group, numerous biofilms appeared yellow due to the green staining of 8DSS-C8-P-113 hydrogel (indicated by arrows), while some dead S. mutans cells were stained red (data not shown). Approximately 99.00%, 99.35%, and 100% S. mutans were killed at the concentration 4×MBC, 8×MBC, and 16×MBC of DPS-C8-P-113, respectively (FIG. 11B). The viability by CFU counts in the 4×MBC DPS-C8-P-113 group [9.16E+05 (CFU mL⁻¹)], 8×MBC DPS-C8-P-113 group [4.38E+05 (CFU mL⁻¹)], and 16×MBC DPS-C8-P-113 group [0 (CFU mL⁻¹)] were significantly lower than the control group [9.20E+07 (CFU mL⁻¹)](p<0.05, FIG. 11B). Approximately 96.59% and 99.98% S. mutans were killed by 8×MBC and 16×MBC of 8DSS-C8-P-113, respectively. The viability by CFU counts in the 8×MBC 8DSS-C8-P-113 group [3.14E+06 (CFU mL⁻¹)] and 16×MBC 8DSS-C8-P-113 group [1.25E+05 (CFU mL⁻¹)] were significantly lower than the control group [9.20E+07 (CFU mL⁻¹)](p<0.05, FIG. 11B). Approximately 90.39%, 99.55%, and 100% S. mutans were killed at the concentration 4×MBC, 8×MBC, and 16×MBC of C8-P-113, respectively (FIG. 11B). All of them at the 8×MBC and 16×MBC presented significant killing to the S. mutans biofilm. However, DPS-C8-P-113 exhibited the most potent inhibitory effects on the eradication of S. mutans biofilm.

Characterization of peptides The characterization of C8-P-113, DPS-C8-P-113, and 8DSS-C8-P-113 was evaluated, with their respective isoelectric points (pI) being 12.15, 12.15, and 6.35. In HEPES (concentration: 1 μM mL⁻¹, pH=7), C8-P-113 and DPS-C8-P-113 were colourless transparent liquids, while 8DSS-C8-P-113 presented a white suspension. After 10 minutes of dissolving in HEPES (pH=7), 16 μM mL⁻¹ C8-P-113 remained a colourless transparent liquid, while DPS-C8-P-113 formed a white suspension at the same concentration. In contrast, 16 μM mL^{-1 8}DSS-C8-P-113 formed a hydrogel. The thick hydrogel adhered to the bottom of an inverted vial. Furthermore, the 2-fold diluted series of 8DSS-C8-P-113 displayed a two-layer status at concentrations of 16, 8, 4, and 2 μM mL⁻¹, respectively.

### TEM Micrographs of Peptides

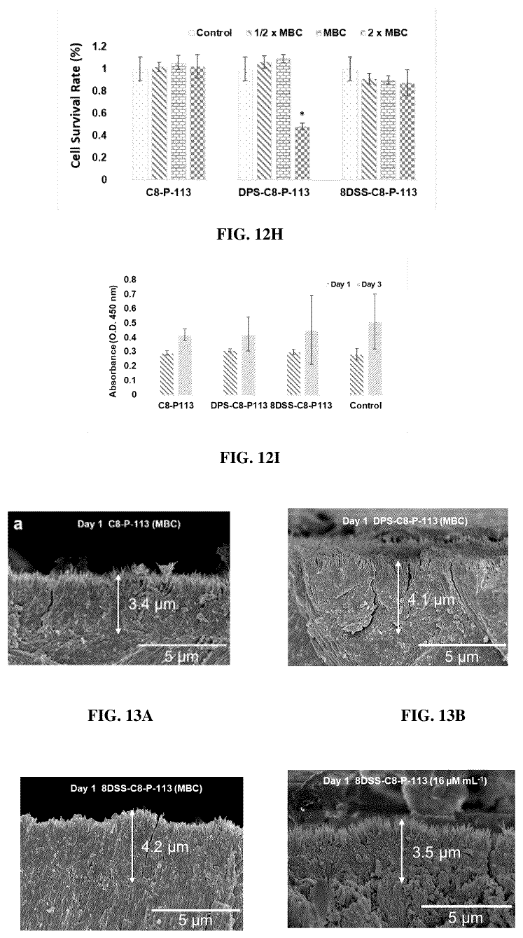

TEM micrographs revealed distinct morphologies for each peptide. C8-P-113 exhibited a spherical particle shape with a diameter of 100 nm (FIG. 12A), while DPS-C8-P-113 had a short linear shape with a length of 100-150 nm and a width of 25-33 nm (FIG. 12B). In contrast, 8DSS-C8-P-113 displayed a three-dimensional fibre network with helical twists, as illustrated in the FIG. 12C. The fibres were about 25-33 nm wide and varied in length, often appearing very long.

### FE-SEM Micrographs of 8DSS-C8-P-113 Hydrogel

After fixation and dehydration, a 3D fibre network of 8DSS-C8-P-113 hydrogel was observed by FE-SEM (FIG. 12D and FIG. 12E). The fibres in the network were approximately 30 nm in width, and the hydrogel exhibited pore sizes with a diameter of around 500 nm.

### Stability of Peptides in Human Saliva

The changes in the remaining percentage (FIG. 12F) and concentration (Table 6) of peptides incubated in human saliva are shown. 11.9% of C8-P-113 remained, 56.2% of DPS-C8-P-113 remained, and 89.4% of 8DSS-C8-P-113 remained intact after being incubated in human saliva at 37° C. for 6 hours.

TABLE 6
Changes of peptide concentration incubated in human saliva at 37° C. during 6 h.

| | Incubation Time | | | | | |
|---|---|---|---|---|---|---|
| | 0 h | 0.5 h | 1 h | 2 h | 4 h | 6 h |
| C8-P-113 | 1 | 0.953111 | 0.860234 | 0.497746 | 0.357845 | 0.119026 |
| (mg mL-1) | | | | | | |
| DPS-C8-P-113 | 1 | 0.981818 | 0.978512 | 0.950413 | 0.538843 | 0.561983 |
| (mg mL-1) | | | | | | |
| 8DSS-C8-P-113 | 1 | 0.994232 | 0.987022 | 0.988464 | 0.899063 | 0.894016 |
| (mg mL-1) | | | | | | |

FIG. 12G illustrates the changes in the remaining percentage of 8DSS-C8-P-113 hydrogel when incubated in flow water at a rate of the 30 mL h-1. After 2 h, 8DSS-C8-P-113 hydrogel maintained approximately 82.15% of its initial status on the tooth surface. The hydrogel's dissolving percentage was approximately 4.46% every 30 min.

These results demonstrate that 8DSS-C8-P-113 exhibits desirable stability in human saliva.

### Cytocompatibility of Peptides

After a 24-hour incubation, there were no significant differences in cell survival rates in all concentrations of C8-P-113, ½×MBC and MBC DPS-C8-P-113, and all concentrations of 8DSS-C8-P-113 when compared to the control group (FIG. 12H). The survival rate of DPS-C8-P-113 at 2×MBC concentration decreased to 48%. Additionally, after 1 and 3 days of incubation, the proliferation of MC3T3-E1 cells on peptide-coated enamel surfaces (at MBC concentration) was not significantly different among all groups (FIG. 12I). Results from fluorescent images (data not shown) were consistent with the quantitative CCK-8 assay. MC3T3-E1 cells were stained green and exhibited a spindle-like morphology in all groups after 3 days of incubation. During the 3-day incubation period, peptide-coated groups showed a significantly increasing number of live cells compared to the control group. Furthermore, the hydrogel formed from 16 μM mL-1 of 8DSS-C8-P-113 was also found to be safe. These results indicate that peptides at MBC concentration exhibit good cytocompatibility and demonstrate appropriate safety for clinical application.

Based on the arrangement and combination of the three functional parts, i.e., P-113 (the AMP), C8 (the competence stimulating peptide), and DPS/8DSS (the domains of improving remineralization), 12 new molecules can be fabricated. However, considering the function of specific antimicrobial activities, the sequence of C8-P-113 is a better design. DPS/8DSS was planned to be in the end of C-terminus or N-terminus to design four molecules, including C8-P-113-DPS, DPS-C8-P-113, C8-P-113-8DSS, 8DSS-C8-P-113. Nevertheless, C8-P-113-DPS could not be synthesized due to the weak stability during fabrication (Table 1). C8-P-113-8DSS has weaker antibacterial activity against S. mutans (Table 2). DPS-C8-P-113 and 8DSS-C8-P-113 have good stability and cytocompatibility, which demonstrates that the domain of improving remineralization in the N-terminus is better than that in the C-terminus. Since 8DSS-C8-P-113 with a concentration of 16 μM mL⁻¹ can self-form hydrogels, the 3D network structure of this status absorbs a large amount of water^{34,35}. Hydrogels are hydrophilic polymers that have properties of flexibility, porosity, soft structure, biodegradability, biocompatibility, low immunogenicity, and ease of usage^{36, 37}. Because of these advantages, they have been applied in many fields, such as drug delivery, tissue engineering and regenerative medicine³⁸. In this study, the 16 μM mL⁻¹ 8DSS-C8-P-113 remained over 80% hydrogel status on the tooth surface even after exposing to a simulated salivary flow environment for 2 h. This safe and stable hydrogel can offer protection to the tooth against damage from bacteria and acid.

### Remineralization Property

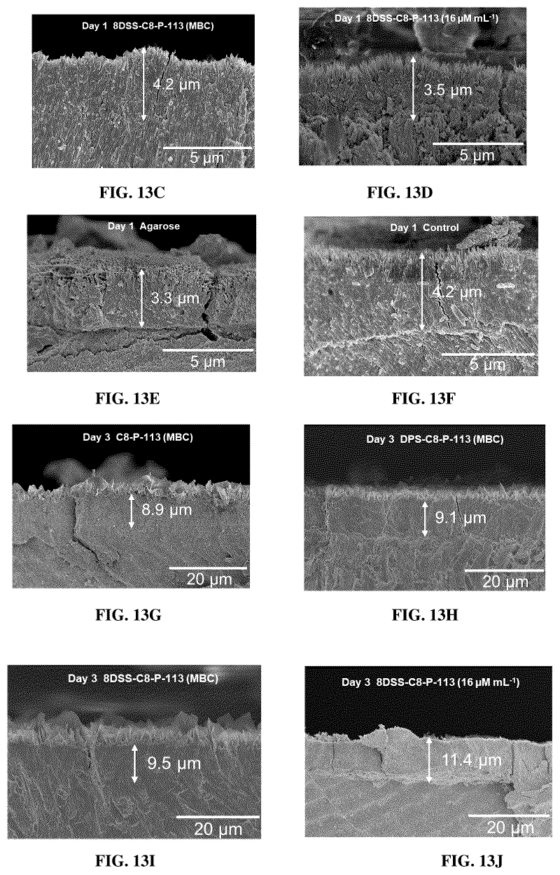

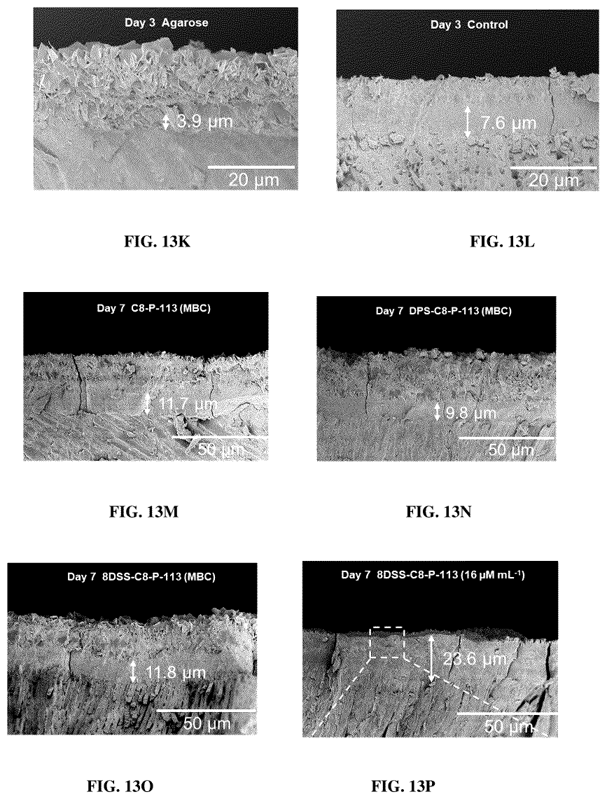

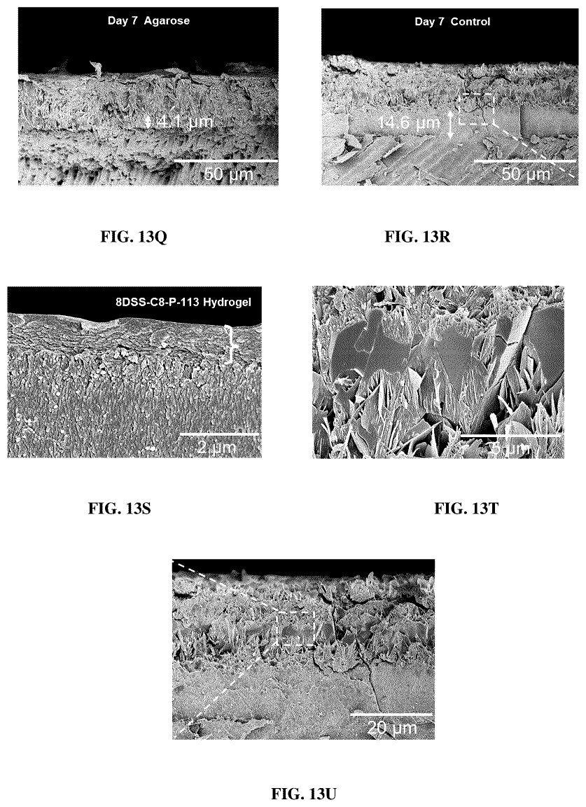

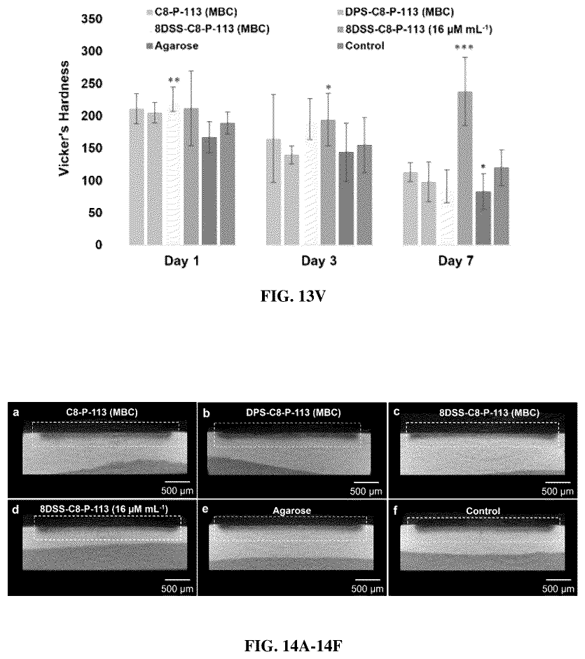

After exposure of enamel surfaces to remineralization solutions for 1 day, the generation of new HA crystals as imaged by FE-SEM were formed on demineralized enamel slices in all groups (FIGS. 13A-13F). Cross-sectional micrographs of the remineralized enamel slices clearly showed a newly regenerated layer tightly conjoined to its underlying natural enamel. The thickness of the regenerated crystal layer on the enamel surface among different groups showed no significant difference, around 4 μm (FIGS. 13A-13F). The crystal macrostructure of the regenerated layer formed needle-like crystals. However, a certain thick plate-like crystals formed on the needle-like crystals on the 3rd day in the different treated groups except 8DSS-C8-P-113 (16 μM mL⁻¹) group (FIGS. 13G-13L). The layer of plate-like crystals became thicker on the 7th day in the different treated groups except 8DSS-C8-P-113 (16 μM mL⁻¹) group (FIGS. 13M-13R). In the 8DSS-C8-P-113 (16 μM mL⁻¹) group, the regenerated crystal macrostructure consistently exhibited needle-like crystals. On the 3rd day, the crystals had a thickness of 11.4 μm, which increased to 23.6 μm by the 7th day.

The mean values of Vicker's hardness of 8DSS-C8-P-113 (MBC) group on the 1st day was 226.94±19.61 HV, which was significantly higher than that of control group on the 1st day (189.77±17.44 HV, **p<0.01) (FIG. 13V).

The mean values of Vicker's hardness of 8DSS-C8-P-113 (16 μM mL⁻¹) group on the 3rd day was 194.78±24.96 HV, which was significantly higher than that of control group on the 3rd day (155.45±43.91 HV, *p<0.05) (FIG. 13V). 8DSS-C8-P-113 (16 μM mL⁻¹) group on the 7th day had the highest mean value of Vicker's hardness (238.89±53.24 HV), whereas Agarose group has the lowest (83.01±28.74 HV), compared with control group (120.24±28.14 HV) on the 7th day (***p<0.001, *p<0.05) (FIG. 13V).

1 ppm fluoride was effective to manipulate forming needle-like (hexagonal prisms structure) instead of porous plate-like HAP nanocrystals³⁹. In order to reduce the side effects of fluoride, 1 ppm fluoride was used in this study. When incubating for one day, the regenerated minerals tended to be needle-like crystals, which is consistent with the findings of Fan et al. However, as the process time prolonged to 7 days, the regenerated minerals tended to be plate-like crystals. Prolonged heating time could influence the aggregation and morphology of HAP through a rearrangement of the built nanostructures, which may alter needle-like to plate-like crystals⁴⁰. Apart from process time, other parameters like Ca/P molar ratio concentration, temperature, pH, or additives also contribute to the final structure of HAP, including needles, plates, and spherical-shaped, with various sizes^{41,42}. Based on the results of this study, 16 μM mL⁻¹ 8DSS-C8-P-113 group still presented excellent needle-like crystals after incubation for 7 days, with the highest microhardness value. In order to investigate the physical effects of hydrogel status of 8DSS-C8-P-113 on remineralization, agarose with hydrogel status was used to be the control in this study. However, agarose hydrogel on tooth surface could not improve the microhardness of regenerated layers due to the formation of a lot amount of plate-like crystals. Hydrogels formed from 16 μM mL^{-1 8}DSS-C8-P-113 may react with calcium ions, phosphate ions, or fluoride ions; therefore, they could be used as an additive to manipulate crystal structure on the tooth surface during remineralization.

### Artificial Caries Model

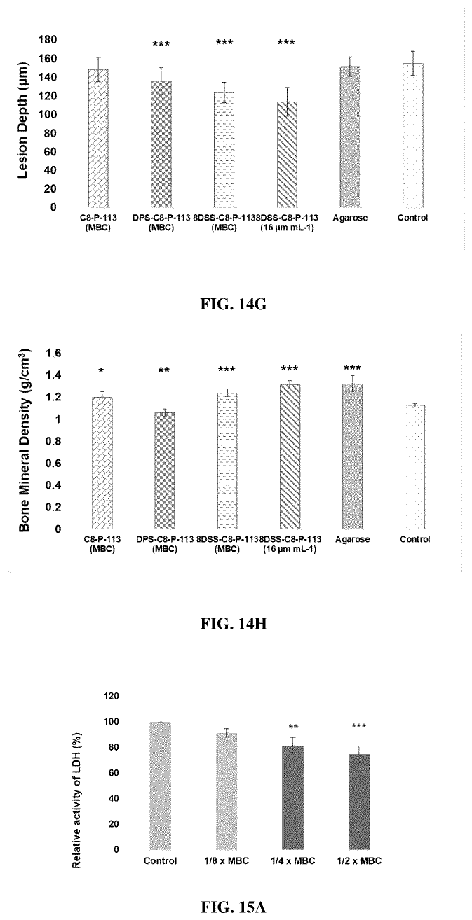

In this study, a closed biofilm model system was used as an artificial caries model⁴³. FIGS. 14A-14F illustrate the cross-section of the lesions on the enamel surface after different surface treatments. There were higher mineral densities in the outermost layer than that below in all groups. Different lesion depths were shown in FIG. 14G. The mean values of lesion depth in the DPS-C8-P-113 (MBC) (136.09±14.41 μm), 8DSS-C8-P-113 (MBC) (123.74±10.82 μm), and 8DSS-C8-P-113 (16 μm mL⁻¹) (113.57±15.24 μm) group were significantly lower than that in the control group (154.71±12.92 μm, ***p<0.001). FIG. 14H shows that the mean values of bone mineral density in the 8DSS-C8-P-113 (MBC) (1.239±0.033 g cm⁻³), 8DSS-C8-P-113 (16 μM mL⁻¹) (1.311±0.038 g cm⁻³), and agarose (1.321±0.073 g cm⁻³) group were significantly higher than that in the control group (1.125±0.016 g cm⁻³, ***p<0.001).

In this study, 8DSS-C8-P-113 (MBC) and 8DSS-C8-P-113 (16 μM mL⁻¹) on the tooth surface showed significant inhibition of demineralization, which might be related to the interaction between peptide and HAP, or the interaction between peptide and S. mutans.

Based on the evaluation of above experiments, the comparison of properties of peptides were listed in the Table 3. 8DSS-C8-P-113 was determined to be the superior AMP, with the specifically targeted anti-S. mutans activity, better stability, better cytocompatibility, higher ability to improve remineralization, and higher ability to inhibit demineralization in the artificial caries model, especially its good self-assembly ability to be a hydrogel status to promote its properties.

The mechanism of antibacterial activity of 8DSS-C8-P-113 To investigate the mechanism of antibacterial activity of 8DSS-C8-P-113, transcriptome analysis of S. mutans exposed to 8DSS-C8-P-113 was conducted. To investigate the mechanism of the hydrogel formation of 8DSS-C8-P-113 and the mechanism of improving remineralization of this peptide, MD analysis was used

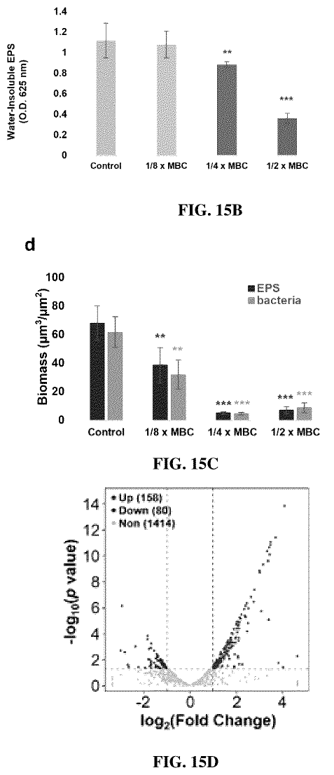

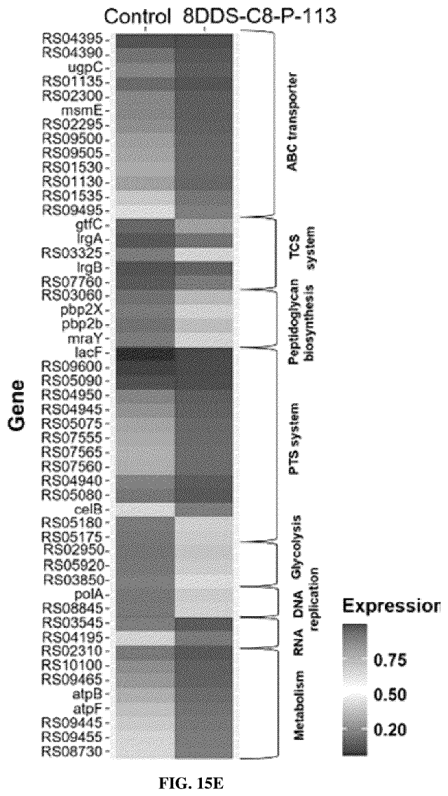

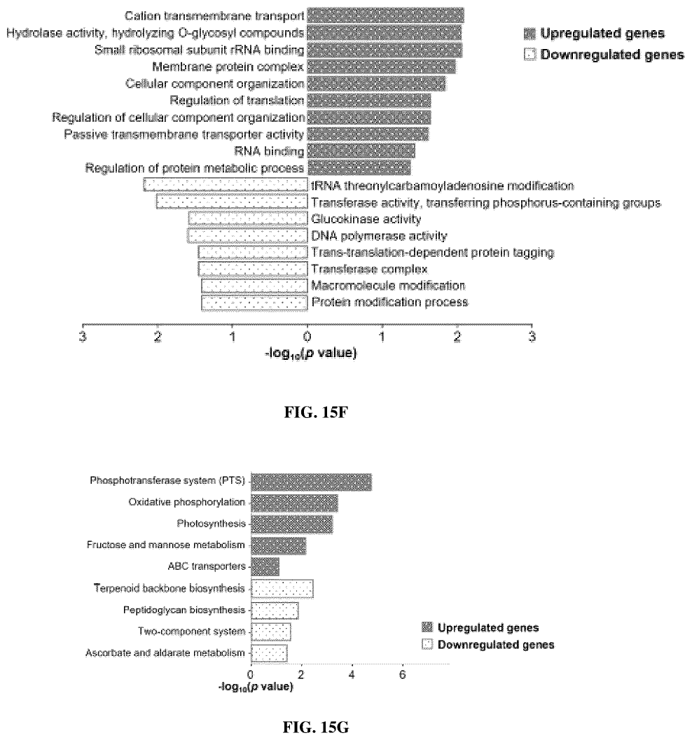

The effects of 8DSS-C8-P113 at sub-MBC levels on acidogenicity were monitored by the LDH activity of the S. mutans culture. The cariogenic virulence factors of S. mutans, such as acidogenicity, acid tolerance, and biofilm formation, play a significant role in causing tooth decay [26]. LDH produced by S. mutans contributes to acidogenicity by converting pyruvate to lactic acid, which leads to tooth lesions. As shown in FIG. 15A, when S. mutans was treated with 8DSS-C8-P113 at ¼×MBC and ½×MBC, its LDH activity was significantly lower than that of control group (**p<0.01, ***p<0.001). The absorbance in the groups treated with 8DSS-C8-P113 at ¼×MBC and ½×MBC significantly reduced, which indicated that the amount of water-insoluble EPS significantly reduced compared with that in the control group (**p<0.01, ***p<0.001, FIG. 15B). Further analysis of biofilms treated with 8DSS-C8-P113 at ⅛, ¼ and ½×MBC revealed a thinner, looser biofilm structure with a significantly reduced biomass of bacteria (green) and EPS matrix (red) compared to the untreated control (data not shown). The quantitative analyses of EPS and biofilm formation showed a significant reduction in the amount water-insoluble EPS with ⅛, ¼ and ½×MBC of 8DSS-C8-P113 (**p<0.01, ***p<0.001, FIG. 15C). EPS produced by bacteria are important components of the biofilm matrix. Various agents have been found to inhibit the activity of S. mutans by affecting the production of LDH and EPS. In this study, the treatment group using 8DSS-C8-P113 showed lower levels of LDH and EPS compared to the control group. This suggests that 8DSS-C8-P113 has the ability to reduce acidogenicity and biofilm formation at sub-MBC levels.

To further explore the underlying molecular mechanisms of 8DSS-C8-P-113 reacting to S. mutans, transcriptome analysis of S. mutans was performed. Compared with the control group, a total of 238 DEGs were identified in the 8DSS-C8-P-113-treated group (|log₂fold change|>1 and P<0.05), including 158 upregulated genes and 80 downregulated genes (FIG. 15D). The expression of genes associated with ATP-binding cassette (ABC) transporters (e.g., RS04395, RS04390, ugpC, msmE, and so on), sugar phosphotransferase system (PTS) (e.g., lacF, RS09600, RS05090, and so on), RNA degradation-related genes (e.g., RS03545 and RS04195) and lipid metabolism-related genes (e.g., RS02310, RS10100 and so on) were upregulated in the 8DSS-C8-P-113 group (FIG. 15E). By contrast, DEGs associated with two-component (TCS) system (e.g., gtfC, lrgA, RS03325, lrgB, and RS07760), peptidoglycan biosynthesis (e.g., RS03060, pbp2X, pbp2b, and mraY), glycolysis (e.g., RS02950, RS05920, RS03850) and DNA replication (e.g., polA and RS04195) were significantly downregulated in the 8DSS-C8-P-113-treated strain. Notably, the expression of glucan synthesis-related gene gtfC was substantially decreased in the 8DSS-C8-P-113 group (FIG. 15E).

AMPs have broad spectrum antimicrobial activities defencing organisms. The principal antibacterial activities have been attributed to the membrane-lytic and intracellular targeting activities but the details have not been elucidated. For example, antifungal activities to Candida Albicans of human peptide Histatin 5 were accredit with multiple intracellular components to induce oxidative stress and mitochondrial malfunction⁴⁹. P-113 killed S. mutans by penetrating bacterial membranes and targeting intracellular components such as DNA¹⁶. These in vitro studies show the associations, yet cannot explore unknown or probable mechanisms. Transcriptomic sequencing is a technique that interprets gene function and gene structure, reveal molecular mechanisms of specific biological processes, and discover unknown and rare transcriptional information⁵⁰. Based on the transcriptomic analysis, the probable anti-S. mutans mechanism of 8DSS-C8-P-113 includes influencing bacterial membranes, inhibiting glycolysis, interfering protein homeostasis and amino acid metabolism, and suppressing DNA replication and oxidation. 8DSS-C8-P-113 may influence the stability or integrity of S. mutans bacterial membranes by upregulating PTS system and ABC transporters, and downregulating TCS system and peptidoglycan biosynthesis of S. mutans. S. mutans has a significant number of transporters of the ABC superfamily. Members of this superfamily are involved in the translocation of a diverse range of molecules across the membrane⁵¹. Some studies showed that ABC transporters and PTS were mainly responsible for the transporting of sugars^{52,53}. ABC transporters are well known in increasing the transcription levels for the transportation of sugars, especially under stress conditions. Additionally, they also function in exporting protons to regulate the intracellular pH^{53,54}. To survive in the complex oral microenvironment, microorganisms use regulatory networks to detect environmental signals and control adaptive responses.

TCS is one of the regulatory networks including QS and antibiotic/bacteriocin production, which typically consists of a membrane associated histidine kinase (VicR) and a cytoplasmic response regulator (VicK)^{52,55}. Virulence factors production of S. mutans is regulated by TCS genes, including biofilm formation, acidogenicity, aciduricity, genetic transformation and bacteriocin production⁵⁶. Both of VicK and VicR upregulate the expression of GTFC enzyme (one of glucosyltransferase enzymes, GTFs) by binding their promoter regions, and GTFC enzyme is encoded by gtfC genes⁵⁵. GTFC enzyme produces a mixture of insoluble and soluble glucans (α-1,6-linked glucose). The insoluble glucans can induce bacterial adhesion on tooth surface and the formation of biofilm, while the soluble glucans can be digested to cause low pH values⁵⁷. In this study, gtfC gene was downregulated in the 8DSS-C8-P-113-treated group. The CSP plays a key role in the regulation of bacterial QS. C8, as a CSP in the 8DSS-C8-P-113, may bind with S. mutans, activate a transmembrane receptor to interfere the QS of S. mutans, and inhibit the function of GTFC enzyme. In the end, 8DSS-C8-P-113 may inhibit S. mutans' adhesion, formation of biofilm, and production of acid by downregulating gtfC gene. For the inhibition of tooth demineralization in the artificial caries model, it is also a probable and reasonable explanation. The cell wall of most bacteria is made of a large polymer peptidoglycan, which forms a mesh-like scaffold outside the plasma membrane. Peptidoglycan synthesis is crucial for the growth of bacterial cell. Penicillin-binding proteins (PBPs) and transpeptidase catalyse the transglycosylation, transpeptidation, and trimming of the peptidoglycan in the last stage of peptidoglycan biosynthesis⁵⁸. S. mutans has five PBPs (PBP1a, PBP1b, PBP2a, PBP2b, and PBP2X) which involve in the peptidoglycan biosynthesis⁵⁹. The gene PBP2X and PBP2b are core-genomes for the class B PBPs, and these PBPs are considered important targets for the beta-lactam antibiotics⁶⁰. In this study, PBP2X and PBP2b gene were downregulated in the 8DSS-C8-P-113-treated group. 8DSS-C8-P-113 might interfere the peptidoglycan biosynthesis of S. mutans to make bacterial cells more prone to osmotic lysis, even to inhibit the growth of S. mutans. S. mutans is the cariogenic pathogen and synthesize EPS from dietary sugars, which can form plaque biofilm and secrete acids to demineralize teeth. Sugars are the major carbohydrate sources of S. mutans. S. mutans can use them to produce ATP through glycolysis, to synthesize bacterial components, and to produce polysaccharides and acids⁶¹. Sugar metabolism is very important for the growth and virulence of S. mutans. The data showed that some genes related to glycolysis were downregulated in the 8DSS-C8-P-113-treated group. 8DSS-C8-P-113 might interfere the glycolysis of S. mutans to decrease the virulence of S. mutans. The decrease of acid may decline injury to tooth, which may be related to the ability of inhibiting demineralization of 8DSS-C8-P-113.

To further investigate the biological function and potential pathways involved in the antibacterial effect of 8DSS-C8-P-113 on S. mutans, Gene Ontology (GO) and Kyoto Encyclopedia of Genes and Genomes (KEGG) pathway enrichment analyses were performed, respectively. GO enrichment analysis showed that the upregulated DEGs were enriched in cation transmembrane transport, hydrolase activity, small ribosomal subunit rRNA binding and membrane protein complex, while the downregulated DEGs were enriched in transferase activity, glucokinase activity, DNA polymerase activity and protein modification process (FIG. 15F, P<0.05). And KEGG enrichment analysis indicated that the upregulated DEGs were implicated in PTS system, oxidative phosphorylation and ABC transporters, while the downregulated DEGs were associated with terpenoid backbone biosynthesis, peptidoglycan biosynthesis, TCS system and ascorbate and aldarate metabolism (FIG. 15G, P<0.05).

The results of GO and KEGG pathway enrichment analyses were consistent with the DEGs analyses. Based on the results, 8DSS-C8-P-113 may bind with S. mutans and activate a transmembrane receptor to interfere the QS of S. mutans by the CSP C8 to inhibit the formation of biofilm, interfere the peptidoglycan biosynthesis of S. mutans to cause osmotic lysis, interfere the glycolysis of S. mutans to decrease the virulence of S. mutans, increase the perforation of cell membrane, and penetrate into the plasm membrane to inhibit DNA polymerase activity and protein modification. Therefore, 8DSS-C8-P-113 inhibits the cariogenic virulence and biological function of S. mutans through multiple pathways.

### Mechanism of Remineralization

After 100 ns of dynamic simulation, a dispersed state of 20 8DSS-C8-P-113 molecules in a solvent box formed stable clusters of hydrogel nanostructures (data not shown) The formation of a hydrogel from peptide molecules was primarily driven by electrostatic interactions, with an average electrostatic energy of −274210.725±1893.481 kJ mol⁻¹ and an average van der Waals energy of −19332.002±2226.747 k mol⁻¹ (data not shown). In the entire system, hydrophobic interactions were the most abundant interactions between peptide molecules, with a mean value of 1135.78. They were followed by hydrogen bonding, with a mean value of 897.10, and pi-pi conjugation interactions were the least prevalent, with a mean value of 64.79 (data not shown). The binding between the two peptide chains was mainly facilitated by hydrogen bonding and hydrophobic interactions. A total of 7 hydrogen bonding interactions were identified between the chains. Asp13, Lys34, Arg35, Arg41, Lys42, and His44 were found to be the primary amino acid residues involved in hydrogen bonding in one peptide chain, while Ser14, Ser15, Asp22, Arg28, and His37 were the main amino acids involved in the other peptide chain. Overall, the analysis results indicate that hydrogen bonding and hydrophobic interactions are the main driving forces behind the aggregation of dispersed peptide molecules into a hydrogel in the entire system, with electrostatic interactions playing a promotional role. Additionally, the hydrogen bonding and hydrophobic interactions between charged amino acid residues, such as serine, aspartic acid, lysine, arginine, and histidine, play a crucial role in this process. From a functional perspective, hydrogen bonding interactions predominantly occur between functional groups that carry either a partial or a full charge.

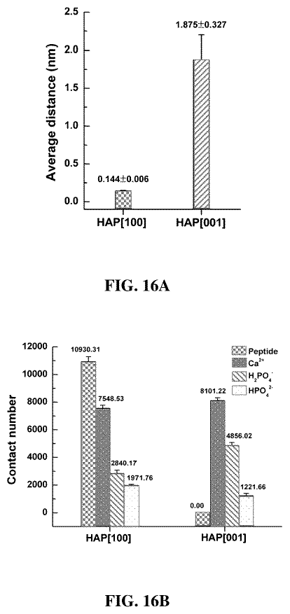

It was observed in the that 8DSS-C8-P-113 hydrogel moved towards the HAP (100) surface and was stably adsorbed through hydrogen bonding interactions between the side chain amino groups of lysine and arginine amino acids in the hydrogel and the HAP surface (data not shown). However, weak interactions between the 8DSS-C8-P-113 hydrogel and the HAP (001) surface prevented the entire peptide cluster from adsorbing onto the surface and exhibiting obvious interactions (data not shown). During the entire simulation process, the average distance between the hydrogel and the HAP (100) surface (0.144±0.006 nm) was shorter than that between the hydrogel and the HAP (001) surface (1.875±0.327 nm) (FIG. 16A). In the system, the contact number between the hydrogel ions and HAP (100) surface was significantly higher than that with the HAP (001) surface (FIG. 16B). The contact number between the free HPO₄²⁻ phosphate ions and the HAP (100) surface was slightly higher than that with the HAP (001) surface. However, the contact number between the free Ca²⁺ ions and HAP (100) surface was lower than that with the HAP (001) surface, which was consistent with the radial distribution results (data not shown). The contact number between the free H₂PO₄⁻ ions and HAP (100) surface was also lower than that with the HAP (001) surface. The data showed that Ca²⁺, H₂PO₄⁻, and HPO₄²⁻ ions were randomly dispersed in a rectangular area 2 nm above 8DSS-C8-P-113 hydrogel in the HAP (100) system before simulation (data not shown). After 100 ns, the hydrogel adsorbed onto the HAP (100) surface, causing the clusters to become looser. Due to the adsorption of 8DSS-C8-P-113 hydrogel molecules, most of the HAP (100) surface area was occupied by the hydrogel molecules, while Ca²⁺, H₂PO₄⁻, and HPO₄²⁻ ions were relatively more concentrated in areas not covered by peptides. Most of Ca²⁺ ions, H₂PO₄⁻, and HPO₄²⁻ adsorbed onto the HAP (001) surface more dispersed and evenly distributed, while the 8DSS-C8-P-113 hydrogel molecules did not adsorb onto the HAP (001) surface after 100 ns of MD simulation (data not shown).

The difference in the adsorption of 8DSS-C8-P-113 hydrogel on the two HAP surfaces affects the adsorption between the ions and HAP surface. Ca2+ and H2PO4-ions are found to aggregate less on the HAP (100) surface. This may due to the adsorption of the 8DSS-C8-P-113 hydrogel occupying most of the surface area of HAP (100), thereby suppressing the adsorption of ions to some extent. HPO42- ions also aggregates less on the HAP (001) surface, possibly because more H2PO4- phosphate ions occupy most of the space on the HAP (001) surface. Therefore, the molecular simulation results indicates that the 8DSS-C8-P-113 hydrogel exhibits minimal adsorption on the HAP (001) surface. As there is no spatial hindrance from the 8DSS-C8-P-113 hydrogel on the HAP (001) surface, calcium and phosphate ions are more likely to aggregate on the surface, leading to the growth of the crystal structure along the (001) direction.

### Schema of Mechanism

Based on the results in this study, the multiple functions of 8DSS-C8-P-113 are summarized as follows. Firstly, numerous of 8DSS-C8-P-113 molecules self-assemble a hydrogel state and adhere to the tooth surface. Secondly, 8DSS-C8-P-113 kills planktonic S. mutans, inhibits S. mutans' adhesion, eradicates S. mutans' biofilm, and reduces S. mutans' virulence. The probable mechanisms of 8DSS-C8-P-113 interfering S. mutans include: 1) It induces cell membrane perforation by up-regulating cation transmembrane transport and ABC-transporter. 2) C8 in 8DSS-C8-P-113 activates a transmembrane receptor, leading to interference with QS and inhibition of biofilm formation by down-regulation of TCS. 3) It interferes with peptidoglycan biosynthesis, resulting in osmotic lysis. 4) It interferes with glycolysis, causing decreased virulence by down-regulating glucokinase activity, as well as ascorbate and aldarate metabolism. 5) 8DSS-C8-P-113 inhibits DNA polymerase activity. 6) 8DSS-C8-P-113 inhibits protein modification. Thirdly, when exposed to the remineralization solution, 8DSS-C8-P-113 hydrogel on the enamel surface induces the adherence of essential ions, including Ca2+, H2PO4-, and HPO42-, to HAP (001). This process facilitates the formation of a needle-shaped remineralization crystal layer between the 8DSS-C8-P-113 hydrogel and the tooth surface. Fourthly, 8DSS-C8-P-113 hydrogel adsorbs onto the HAP (100) surface, effectively inhibiting the adhesion of S. mutans and subsequently reducing tooth demineralization.

The specific targeting agent 8DSS-C8-P-113, at a concentration of 2 μM mL-1, can be used as a mouthwash for personal care at home to effectively eliminate planktonic S. mutans without disrupting the oral normal flora, such as S. gordonii and S. sanguinis. For professional care at dental clinic, 8DSS-C8-P-113 with the concentration of 8 μM mL-1 can be employed in the form of varnish to prevent S. mutans' adhesion. The hydrogel formed from 8DSS-C8-P-113 with the concentration of 16 μM mL-1 can be applied directly to eradicate S. mutans' biofilm, safeguard teeth against acid injury, and facilitate the repair of demineralized hard tissue.

### Suppression of Dental Caries in a Rat Caries Model

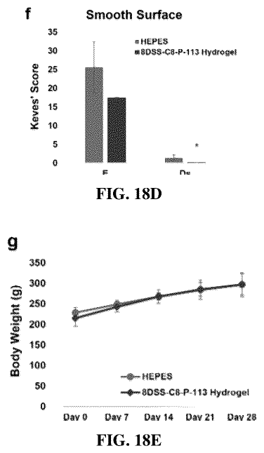

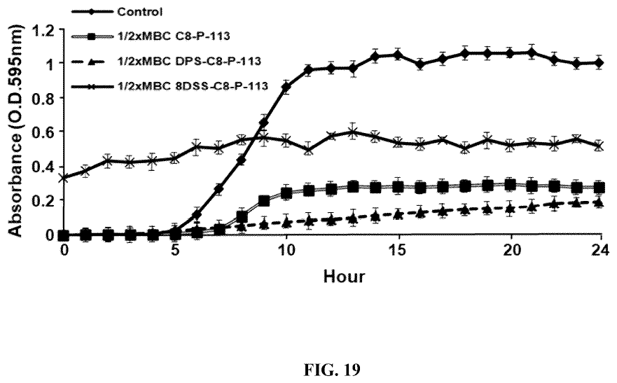

The preventive efficacy of 8DSS-C8-P-113 for dental caries in vivo was determined using a rat dental caries model. The experimental procedure for the rat dental caries model is shown in FIG. 18A. Compared to the control group treated with HEPES only, rats treated with 8DSS-C8-P-113 presented a significant decrease in S. mutans CFU from dental plaques (P<0.05) (FIG. 18B). Compared to the HEPES group, the volume of enamel lesions and dentin lesions on rat teeth was reduced in the 8DSS-C8-P-113 group (data not shown). Enamel and dentin lesions were more severe in the HEPES group. Enamel caries was observed on the buccal, lingual, and proximal surfaces, and the lesions on the sulcal surface were deeper than half dentin thickness (data not shown). On the sulcal surface, the Keyes' scores were significantly lower in the 8DSS-C8-P-113 group than in the HEPES group (E and Ds: P<0.05, Dm: P<0.01) (FIG. 18C). On the smooth surface, the Keyes' scores of Ds were significantly lower in the 8DSS-C8-P-113 group than in the HEPES group (Ds: P<0.05) (FIG. 18D). All rats survived without significant differences in body weights observed between HEPES and 8DSS-C8-P-113 groups (FIG. 18E). No signs of inflammatory reaction, proliferative changes, or necrosis were observed in the histopathology analysis of both groups (data not shown). These results demonstrate that 8DSS-C8-P-113 can effectively reduce the severity of caries in the rat caries model. Along with its good biocompatibility, it can be a potent and safe agent for clinical application in humans. It is important to acknowledge the limitations of this study, which include the absence of mechanical property data for 8DSS-C8-P-113 and the lack of in vivo investigations on its remineralization ability. The concentration of 16 μM mL⁻¹ was selected in the rat caries model specifically to evaluate the biocompatibility of 8DSS-C8-P-113 at a higher concentration. Additionally, while the experiment showed a reduction in caries severity in the treatment group, there were still instances of dental caries in the extremely cariogenic environment, which may also have been due to operational difficulties in the small rat mouth. Therefore, further investigations are necessary to explore the various factors that may influence the efficacy of 8DSS-C8-P-113 and to address the limitations of this study.

### CONCLUSIONS

A multi-functional agent 8DSS-C8-P-113 has been designed and synthesized to possess the specifically targeted killing properties and in situ remineralization/anti-demineralization capabilities. In this study, 2 μM mL⁻¹ of 8DSS-C8-P-113 effectively eliminates planktonic S. mutans without disrupting S. gordonii and S. sanguinis. Revealed by transcriptome analyses, the agent activates a transmembrane receptor by the specifically targeted domain C8, induces cell membrane perforation, interferes with peptidoglycan biosynthesis, and inhibits DNA polymerase activity and protein modification. At a concentration of 8 μM mL⁻¹, 8DSS-C8-P-113 exhibits the ability to prevent S. mutans' adhesion by interfering with glycolysis, thereby reducing virulence. When used at a higher concentration of 16 μM mL⁻¹, 8DSS-C8-P-113 eradicates S. mutans' biofilm by interfering with QS and inhibiting the production of EPS in S. mutans. Additionally, 16 μM mL⁻¹ of 8DSS-C8-P-113 inhibits demineralization by suppressing the production of LDH in S. mutans and self-forming a hydrogel state on the tooth surface, which provides protection against acid injury. Further disclosed by MD analyses, 8DSS-C8-P-113 induces the binding of calcium and phosphate ions to the HAP (001) surface, resulting in the formation of a needle-shaped remineralization crystal layer between the 8DSS-C8-P-113 hydrogel and the tooth surface. This process promotes tooth remineralization while mimicking natural bio-mineralization. In summary, this bioactive AMP 8DSS-C8-P-113 exhibits multiple functions, including specifically targeted killing of S. mutans, reduction of S. mutans' virulence, protecting tooth surface against acid injury, and induction of in situ bio-mimic remineralization. The practical efficacy and safety of 8DSS-C8-P-113 hydrogel have been demonstrated in the rat caries model. Importantly, due to the self-assembly gel capability of 8DSS-C8-P-113, it allows for easy and user-friendly application by operators. These properties suggest that 8DSS-C8-P-113 has the potential to become a safe and effective therapeutic agent for the prevention and treatment of dental caries.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto. It is understood that the disclosed methods and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a step is disclosed and discussed and a number of modifications that can be made to a number of components including the step are discussed, each and every combination and permutation of step and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B—F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B—F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in algorithms or methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions.

Those skilled in the art will recognize, or be able to ascertain using no more than routine experimentation, many equivalents to the specific embodiments of the method and compositions described herein. Such equivalents are intended to be encompassed by the following claims.

We claim:

1. An isolated peptide comprising an antimicrobial domain, a mineralization domain and optionally, a tooth-binding domain,
wherein the antimicrobial domain comprises a histatin 5 protein or a portion thereof,
wherein the remineralization domain comprises phosphoserines or DSSDSSDSSDSSDSSDSSDSSDSS (8DSS) (SEQ ID NO: 11), and
wherein the tooth-binding domain comprises TFFRLFNR (C8) (SEQ ID NO:13).

2. The peptide of claim 1, wherein the peptide is selected from the group consisting C8-P-113 (SEQ ID NO:6), DPS-C8-P-113 (SEQ ID NO:8) or 8DSS-C8-P-113 (SEQ ID NO: 10), or a sequence having at least 90% sequence identity to C8-P-113, DPS-C8-P-113 or 8DSS-C8-P-113.

3. The peptide of claim 1, wherein the phosphoserine domain comprises two or more phosphorylated serines.

4. The peptide of claim 1, wherein the phosphoserine domain comprises two or more phosphorylated serines.

5. The peptide of claim 1, wherein the peptide is: (a) C8-P-113 (SEQ ID NO:6), or a sequence having at least 90% sequence identity to C8-P-113; (b) DPS-C8-P-113 (SEQ ID NO:8), or a sequence having at least 90% sequence identity to DPS-C8-P-113; (c) DPS-C8-P-113 (SEQ ID NO:8), or a sequence having at least 90% sequence identity to DPS-C8-P-113; or (d) 8DSS-C8-P-113 (SEQ ID NO: 10), or a sequence having at least 90% sequence identity to 8DSS-C8-P-113.

6. An oral care composition or device comprising the isolated peptide of claim 1.

7. The oral care composition or device of claim 6 comprising a plurality of copies of the isolated peptide, optionally, further comprising a pharmaceutically acceptable excipient, preferably wherein the excipient is suitable for administration to oral cavity.

8. The oral care composition or device of claim 7, wherein the composition or device is selected from mouthwash, toothpaste, dental cream, oral spray, a rinse, a gargle, a gum, a tablet, an oral lozenge, dental floss, a tooth whitening strip or solution, a cleaning solution, a toothpick, or a toothbrush.

9. A method of preventing or reducing bacteria from teeth in a subject comprising:
comprising contacting the subject's teeth and/or oral cavity with the peptide,
composition of claim 1.

10. The method of claim 9, wherein the bacteria comprise S. mutans, Actinomyces spp., or Atopobium spp.

11. The method of claim 10, wherein S. mutans comprise planktonic S. mutans, S. mutans' adhesion, or S. mutans biofilm.

12. The method of claim 11, wherein planktonic S. mutans are eliminated with at least 2 μM mL⁻¹ of 8DSS-C8-P-113.

13. The method of claim 12, wherein S. mutans' adhesion is prevented with at least 8 μM mL⁻¹ of 8DSS-C8-P-113.

14. The method of claim 13, wherein S. mutans' biofilm is eliminated with at least 16 μM mL⁻¹ of 8DSS-C8-P-113.

15. The method of claim 12, wherein preventing or eliminating bacteria comprises cell membrane perforation, interference with quorum sensing (QS), interference with glycolysis, DNA polymerase inhibition, protein modification inhibition, or any combination thereof.

16. The method of claim 12, wherein: (a) the composition is applied to enamel and/or dentin of a tooth; (b) the composition is bonded to enamel and/or dentin surface by dental adhesives; and/or (c) the antimicrobial compound is FAP-LPAA.

17. The method of claim 16, wherein FAP-LPAA eliminates or prevents a bacterial infection in a subject.

18. The method of claim 17, wherein the bacterial infection comprises an infection of the tooth or gum.

19. The method of claim 18, wherein: (a) FAP-LPAA is administered to one or more teeth of the subject; and/or (b) the subject has dental carries, tooth decay, enamel hypoplasia, tooth lesions, bone-filling material, or any combination thereof.

20. The method of claim 19, wherein FAP-LPAA is applied directly to the tooth, bone, or enamel and bonded to the tooth, enamel, or bone via a dental adhesive.


SEQUENCE LISTING (16 sequences)

SEQ ID NO: 1 - protein, 24 aa, synthetic construct
DSHAKRHHGYKRKFHEKHHSHRGY

SEQ ID NO: 2 - protein, 12 aa, synthetic construct
AKRHHGYKRKFH

SEQ ID NO: 3 - protein, 51 aa, synthetic construct
MKFFVFALILALMLSMTGADSHAKRHHGYKRKFHEKHHSHRGYRSNYLYDN

SEQ ID NO: 4 - protein, 14 aa, synthetic construct
  MOD_RES 13: note = Serine is phosphorylated
  MOD_RES 14: note = Serine is phosphorylated
AKRHHGYKRKFHSS

SEQ ID NO: 5 - protein, 27 aa, synthetic construct
AKRHHGYKRKFHSKHKGGKHKGGKHKG

SEQ ID NO: 6 - protein, 13 aa, synthetic construct
  MOD_RES 13: note = 3,4-dihydroxyphenethylamine conjugated to D
AKRHHGYKRKFHD

SEQ ID NO: 7 - protein, 25 aa, synthetic construct
  MOD_RES 1: note = Serine is phosphorylated
SDSHAKRHHGYKRKFHEKHHSHRGY

SEQ ID NO: 8 - protein, 22 aa, synthetic construct
  MOD_RES 1: note = Serine is phosphorylated
  MOD_RES 2: note = Serine is phosphorylated
SSTFFRLFNRAKRHHGYKRKFH

SEQ ID NO: 9 - protein, 44 aa, synthetic construct
TFFRLFNRAKRHHGYKRKFHDSSDSSDSSDSSDSSDSSDSSDSS

SEQ ID NO: 10 - protein, 44 aa, synthetic construct
DSSDSSDSSDSSDSSDSSDSSDSSTFFRLFNRAKRHHGYKRKFH

SEQ ID NO: 11 - protein, 24 aa, synthetic construct
DSSDSSDSSDSSDSSDSSDSSDSS

SEQ ID NO: 12 - protein, 20 aa, synthetic construct
TFFRLFNRAKRHHGYKRKFH

SEQ ID NO: 13 - protein, 14 aa, synthetic construct
  MOD_RES 1: note = Serine is phosphorylated
  MOD_RES 2: note = Serine is phosphorylated
SSAKRHHGYKRKFH

SEQ ID NO: 14 - protein, 36 aa, synthetic construct
AKRHHGYKRKFHDSSDSSDSSDSSDSSDSSDSSDSS

SEQ ID NO: 15 - protein, 36 aa, synthetic construct
DSSDSSDSSDSSDSSDSSDSSDSSAKRHHGYKRKFH

SEQ ID NO: 16 - protein, 22 aa, synthetic construct
  MOD_RES 21: note = Serine is phosphorylated
  MOD_RES 22: note = Serine is phosphorylated
TFFRLFNRAKRHHGYKRKFHSS